(19) United States
(12) Patent Application Publication
(10) Pub. No.: US 2024/0384261 A1
(43) Pub. Date: Nov. 21, 2024

(54) METHODS, COMPOSITIONS, AND KITS FOR PREPARING SEQUENCING LIBRARY

(71) Applicants: Guoliang FU, Abingdon Oxfordshire (GB); GENEFIRST LIMITED, Abingdon Oxfordshire (GB)

(72) Inventors: Guoliang FU, Abingdon Oxfordshire (GB); Thomas DUNWELL, Abingdon Oxfordshire (GB)

(21) Appl. No.: 18/569,672

(22) PCT Filed: Jun. 14, 2022
(86) PCT No.: PCT/GB2022/051492

§ 371 Date: Dec. 13, 2023

(30) Foreign Application Priority Data
Jun. 14, 2021 (GB) 2108427.2

(51) Int. Cl. C12N 15/10

(52) U.S. Cl. CPC C12N 15/1068

(57) ABSTRACT

This invention relates to methods, compositions and kits for processing a target nucleic acid from one or more samples involving linear amplification and tagging two strands of target sequence. A sequencing library is made from the processed nucleic acids suitable for massive parallel sequencing and comprises a plurality of double-stranded nucleic acid molecules.

### BACKGROUND OF THE INVENTION

Next-generation DNA sequencing is continuing to revolutionise clinical medicine and has had an immeasurable impact on basic research. However, while this technology has the capacity to generate hundreds of billions of nucleotides of DNA sequence information in a single experiment, however, an inherent error rate of ˜1% results in hundreds of millions of sequencing mistakes. These scattered errors become extremely problematic when “deep sequencing” genetically heterogeneous mixtures, such as tumours or mixed microbial populations.

To overcome limitations in sequencing accuracy, several methods have been reported. Duplex sequencing (Schmitt, et al PNAS 109: 14508-14513) is one of them. This approach greatly reduces errors by independently tagging and sequencing each of the two strands of a DNA duplex. As the two strands are complementary, true mutations are found at the same position in both strands. In contrast, PCR and sequencing errors result in mutations in only one strand and can thus be discounted as technical error. Another approach called Safe-Sequencing System (“Safe-SeqS) was described by Kinde et al (PNAS 2011; 108(23):9530-5). The keys to this approach are (i) assignment of a unique identifier (UID) to each template molecule, (ii) amplification of each uniquely tagged template molecule to create UID families, and (iii) redundant sequencing of the amplification products. PCR fragments with the same UID are considered mutant (“supermutants”) only if <95% of them contain an identical mutation.

U.S. Pat. Nos. 8,722,368B2, 8,685,678B2, 8,742,606 describe methods of sequencing polynucleotides attached with a degenerate base region to determine/estimate the number of different starting polynucleotides. However, these methods do not compare sequence information of the original two strands and involve ligating and PCR to attach degenerate base region. U.S. Pat. No. 8,742,606B2, and WO2017066592A1, and Quan Peng (Scientific Reports, 2019 Mar. 18; 9(1):4810. doi: 10.1038/s41598-019-41215-z) discuss methods of coupling ligation to double strand DNA together with targeted amplification to generate information on mutations from both strands of starting material.

Another method, ATOM-Seq (WO2018193233A1) allows for a ligation independent method which uses polymerase based tagging of input material which allows for identification of mutations in both strands of starting material. Targeted next generation sequencing often involves the analysis of large complex fragments and this is achieved by multiplex PCR (the simultaneous amplification of different target DNA sequences in a single PCR reaction). Results obtained with multiplex PCR however are often complicated by artefacts of the amplification products. These include false negative results due to reaction failure and false-positive results (such as amplification of spurious products) due to non-specific priming events. Since the possibility of non-specific priming increases with each additional primer pair, conditions must be modified as necessary as individual primer sets are added.

### SUMMARY OF THE INVENTION

This invention relates to methods, compositions and kits for making a non-specific or targeted enriched sequencing library from one or more samples involving one or more initial steps of linear amplification from one or both strands of a target polynucleotide using one or more opposing primers in the presence of an unusual nucleotide during one or more amplification steps, the unusual nucleotide will be able to significantly inhibit the ability of the opposing primers to generate exponential PCR products but has little to no inhibition in the efficiency of the generation of linear amplification products while using a polymerase which is able to incorporate the unusual nucleotide into a modified complementary strand but not be able to use this as a template. The generated sequencing library is suitable for massive parallel sequencing and comprises a plurality of double-stranded nucleic acid molecules.

Disclosed is a method of processing target nucleic acids comprising
(a) providing a reaction mixture(s), each reaction mixture comprising a first polymerase, none or one or more of any of the four standard nucleotides: deoxyadenosine triphosphate (dATP), deoxythymidine triphosphate (dTTP), deoxyguanosine triphosphate (dGTP), and deoxycytidine triphosphate (dCTP), an unusual nucleoside triphosphates and a first primer(s), wherein the polymerase is capable of extending a primer using the target nucleic acids as templates, or in a primer independent manor, and incorporating the unusual nucleotide into extension products to produce modified complementary strands, and is incapable of efficiently making a further copy using the modified complementary strand as template for extension of primers in the opposite orientation, wherein the unusual nucleoside triphosphate is distinct from the four standard nucleotides, and is capable of being incorporated into new strands but cannot being copied as template by said first DNA polymerase; and
(b) performing cycles of extension reactions of the primer and target nucleic acid template to produce a copy or multiple copies of modified complementary strands;

The method may further comprise step (c) adding a second polymerase, which may be a DNA polymerase, which is capable of using the modified complementary strand as template; and
(d) amplifying or replicating the modified complementary strands using the second DNA polymerase.

### DETAILED DESCRIPTION

To facilitate an understanding of the invention, a number of terms are defined below.

As used herein, a “sample” refers to any substance containing or presumed to contain nucleic acids and includes a sample of tissue or fluid isolated from an individual or individuals. Particularly, the nucleic acid sample may be obtained from an organism selected from viruses, bacteria, fungi, plants, and animals. Preferably, the nucleic acid sample is obtained from a mammal. In a preferred embodiment of this invention, the mammal is human. The nucleic acid sample can be obtained from a specimen of body fluid or tissue biopsy of a subject, or from cultured cells. The body fluid may be selected from whole blood, serum, plasma, urine, sputum, bile, stool, bone marrow, lymph, semen, breast exudate, bile, saliva, tears, bronchial washings, gastric washings, spinal fluids, synovial fluids, peritoneal fluids, pleural effusions, and amniotic fluid. A “individual sample” may be a single cell, which can be one T cell or one B cell, while the plurality of samples may be many blood cells in a blood sample.

As used herein, the term “nucleotide sequence” refers to either a homopolymer or a heteropolymer of deoxyribonucleotides, ribonucleotides or other nucleic acids, or any combination of nucleic acids.

As used herein, the term “nucleotide” generally refers to the monomer components of nucleotide sequences even though the monomers may be nucleoside and/or nucleotide analogues, and/or modified nucleosides such as amino modified nucleosides in addition to nucleotides. In addition, “nucleotide” also includes “nucleoside triphosphate” and non-naturally occurring analogue structures which may be naturally occurring or have been developed in selective or targeted approaches. The term “unusual nucleotide” and “nucleotide” may be used interchangeably with the term “unusual nucleotide” preferentially used in context of the present invention and may be used to describe any nucleotide which is in anyway functionally or chemically different from the four standard deoxynucleoside triphosphate (dNTPs) of deoxyadenosine triphosphate (dATP), deoxythymidine triphosphate (dTTP), deoxyguanosine triphosphate (dGTP) and deoxycytidine triphosphate (dCTP).

As used herein, the term “nucleic acid” refers to at least two nucleotides covalently linked together. A nucleic acid of the present invention will generally contain phosphodiester bonds, although in some cases nucleic acid analogues are included that may have alternate backbones. Nucleic acids may be single-stranded or double-stranded, as specified, or contain portions of both double-stranded and single-stranded sequence. The nucleic acid may be DNA, both genomic and cDNA, RNA, DNA, DNA and RNA mixtures, or, DNA-RNA hybrids, where the nucleic acid contains any combination of deoxyribo- and ribo-nucleotides, and any combination of bases, including uracil, adenine, thymine, cytosine, guanine, inosine, xathanine, hypoxathanine, etc. Reference to a “DNA sequence” or “RNA Sequence” can include both single-stranded and double-stranded DNA or RNA. A specific sequence, unless the context indicates otherwise, refers to the single stranded DNA or RNA of such sequence, the duplex of such sequence with its complement (double stranded DNA or RNA) and/or the complement of such sequence.

As used herein, the “polynucleotide” and “oligonucleotide” are types of “nucleic acid”, and generally refer to primers, oligomer fragments to be detected. There is no intended distinction in length between the term “nucleic acid”, “polynucleotide” and “oligonucleotide”, and these terms will be used interchangeably. “Nucleic acid”, “DNA” and similar terms also include nucleic acid analogues. The oligonucleotide is not necessarily physically derived from any existing or natural sequence but may be generated in any manner, including chemical synthesis, enzymatically, DNA replication, reverse transcription or any combination thereof.

As used herein, the terms “target sequence”, “target nucleic acid”, “target nucleic acid sequence”, “target nucleic acid sequence” and “nucleic acids of interest” are used interchangeably and refer to a desired region which is to be either amplified, detected or both, or is the subject of hybridization with a complementary oligonucleotide, polynucleotide, e.g., a blocking oligomer, or the subject of a primer extension process. The target sequence can be composed of DNA, RNA, analogues thereof, or any combinations thereof. The target sequence can be single-stranded or double-stranded. In primer extension processes, the target nucleic acid which forms a hybridization duplex with the primer may also be referred to as a “template. A template serves as a pattern for the synthesis of a complementary polynucleotide. A target sequence for use with the present invention may be derived from any living or once living organism, including but not limited to prokaryotes, eukaryotes, plants, animals, and viruses, as well as synthetic and/or recombinant target sequences, it may also be a mixture of nucleic acids such that target nucleic acid is a subset of the total nucleic acids.

“Primer” as used herein may be used describe, one or more than one primer or a set or plurality of multiple primers and refers to an oligonucleotide(s), whether occurring naturally or produced synthetically. The multiple primers in a set may have different sequences and hybridise to multiple different locations. The terms “first primer”, “a set of first primers” and “a first set of primers” are interchangeable, and the same applies to terms “second primer”. A “Primer” can be functionally described as a molecule capable of acting as a point of initiation of synthesis when placed under conditions in which synthesis of a primer extension product would be expected to occur, which is complementary to a nucleic acid strand is induced i.e., in the presence of nucleotides and an agent for polymerization such as DNA polymerase and at a suitable temperature and in a suitable buffer. Such conditions include the presence of one or more, two or more, three or more, or four or more different deoxyribonucleoside triphosphates which may include but is not limited to deoxyadenosine triphosphate (dATP), deoxythymidine triphosphate (dTTP), deoxyguanosine triphosphate (dGTP) and deoxycytidine triphosphate (dCTP) or suitable additional or replacement nucleotides, unusual nucleotides, and, a polymerization-inducing agent such as DNA polymerase and/or RNA polymerase and/or reverse transcriptase, in a suitable buffer (“buffer” includes substituents which are cofactors, or affect pH, ionic strength, etc.), and at a suitable temperature. The primer is preferably single-stranded for maximum efficiency in amplification. The primers herein are selected to be substantially complementary to a strand of each specific sequence to be amplified. This means that the primers must be sufficiently complementary to hybridize with their respective strands. One or more regions of non-complementary sequence may be attached to the 5′-end of the primer (5′ tail portion) or in the primer (bulge portion), with the remainder of the primer sequence being complementary to the desired section of the target base sequence. Commonly, the primers are complementary, except when non-complementary nucleotides may be present at a predetermined primer terminus or middle region as described. In another expression, the primers herein are selected to be substantially identical to a strand of each specific sequence to be amplified. This means that the primers must be sufficiently identical to one strand, so that they can hybridize with their respective other strands.

As used herein, the term “complementary” refers to the ability of two nucleotide sequences, either randomly or by design, to bind in a sequence complementary dependent manor to each other by hydrogen bonding through their purine and/or pyrimidine bases according to the usual Watson-Crick rules for forming duplex nucleic acid complexes. It can also refer to the ability of nucleotide sequences that may include modified nucleotides or analogues of deoxyribonucleotides and ribonucleotides, or combinations thereof, to bind sequence-specifically to each other by other than the usual Watson Crick rules to form alternative nucleic acid duplex structures.

As used herein, the term “hybridization” and “annealing” are interchangeable, and refers to the process by which two nucleotide sequences complementary to each other, either partially or fully, bind together to form a duplex sequence or segment.

The terms “duplex” and “double-stranded” are interchangeable, meaning a structure formed as a result of hybridization between two complementary sequences of nucleic acids. Such duplexes can be formed by the complementary binding of two DNA segments to each other, two RNA segments to each other, or of a DNA segment to an RNA segment, or two segments composed of a mixture of RNA and DNA to one another, the latter structure being termed as a hybrid duplex. Either or both members of such duplexes can contain modified nucleotides and/or nucleotide analogues as well as nucleoside analogues. As disclosed herein, such duplexes can be formed as the result of binding of one or more blocking oligonucleotides to a sample sequence. The duplex may be partially or completely complementary and may be partially or fully double stranded.

As used herein, the terms “wild-type nucleic acid”, “normal nucleic acid”, “nucleic acid with normal nucleotides”, “wild-type”, “normal”, “wild-type DNA” and “wild-type template” are used interchangeably and refer to a polynucleotide which has a nucleotide sequence that is considered to be normal or unaltered.

As used herein, the term “mutant polynucleotide”, “mutant nucleic acid”, “variant nucleic acid”, and “nucleic acid with variant nucleotides”, refers to a polynucleotide which has a nucleotide sequence that is different from the expected nucleotide sequence of the corresponding wildtype polynucleotide. The difference in the nucleotide sequence of the mutant polynucleotide as compared to the wild-type polynucleotide is referred to as the nucleotide “mutation”, “variant nucleotide”, “variant” or “variation.” The term “variant nucleotide(s)” also refers to one or more nucleotide(s) substitution(s), deletion(s), insertion(s), methylation(s), and/or modification changes.

“Amplification” as used herein denotes the use of any amplification procedures to increase the concentration or copy number of a particular nucleic acid sequence within a mixture of nucleic acid sequences. Amplification can be one or more round of linear amplification, one or more rounds of exponential amplification or a combination thereof.

“Replication” or “replicate” as used herein denotes making a complementary copy of a polynucleotides which is a template for polymerase extension. Many rounds of replication result in amplification.

The terms “reaction mixture”, “amplification mixture” or “PCR mixture” as used herein refer to a mixture of components necessary to amplify at least one product from nucleic acid templates. The mixture may comprise one or more nucleotides (dNTPs), a polymerase (thermostable or not thermostable), primer(s), and a plurality of nucleic acid templates and other unusual nucleotide(s) necessary for the disclosed invention. The mixture may further comprise a Tris buffer, a monovalent salt and Mg²⁺. The concentration of each component, apart from the unusual nucleotide as necessary for the disclosed invention, is well known in the art and can be further optimized by an ordinary skilled artisan.

The terms “amplified product” or “amplicon” refer to a fragment of DNA or RNA amplified by a polymerase a primer, pool of primer, a pair of primers, a pool of pairs of primers or any combination thereof in an amplification method.

The terms “primer extension product” refer to a fragment of DNA or RNA extended by a polymerase using one or a pair of primers in a reaction, which may involve one pass extension, for example first strand cDNA synthesis, or two pass extension, for example double strand cDNA syntheses, or many cycles of extension, which may be a PCR.

The term “compatible” refers to a primer sequence or a portion of primer sequence which is identical, or substantially identical, complementary, substantially complementary or similar to a PCR primer sequence/sequencing primer sequence used in a massive parallel sequencing platform.

The practice of the present invention will employ, unless otherwise indicated, conventional techniques of molecular biology, microbiology and recombinant DNA techniques, which are within the skill of a person skilled in the art. All patents, patent applications, and publications mentioned herein, both supra and infra, are hereby incorporated by reference.

The present invention provides a method of processing target nucleic acids comprising
(a) providing a reaction mixture(s), each reaction mixture comprising a first polymerase, one or more unusual nucleoside triphosphates and a first primer, wherein the polymerase is capable of extending a primer using the target nucleic acids as templates and incorporating the unusual nucleotide into extension products to produce modified complementary strands, and is incapable of efficiently making a further copy using the modified complementary strand as template for extension of primers in the opposite orientation, wherein the unusual nucleoside triphosphate is distinct from the four standard nucleotides (deoxyadenosine triphosphate (dATP), deoxythymidine triphosphate (dTTP), deoxyguanosine triphosphate (dGTP), and deoxycytidine triphosphate (dCTP), and is capable of being incorporated into new strands; and
(b) performing one pass extension or cycles of extension reactions of the first primer on target nucleic acid template to produce copy of modified complementary strands, which cannot efficiently be served as template for further copying in the reaction using the first polymerase, even the opposite primers capable of hybridising to the modified complementary strands are present, because the modified complementary strand containing incorporated unusual nucleotides is a poor template for the first polymerase to replicate.

The method may further comprise:
(c) adding a second polymerase which is capable of using the modified complementary strand as template; and
(d) replicating or amplifying the modified complementary strands using the second polymerase. In this step, the original strands may also be replicated or amplified.

The present invention provides a method of processing target nucleic acids comprising
(a) providing a reaction mixture(s), each reaction mixture comprising a first polymerase, four or more different nucleoside triphosphates including one or more unusual nucleoside triphosphates and a first primer, wherein the polymerase is capable of extending a primer using the target nucleic acids as templates and incorporating the unusual nucleotide into extension products to produce modified complementary strands, and is incapable of efficiently making a further copy using the modified complementary strand as template for extension of primers in the opposite orientation, wherein the unusual nucleoside triphosphate is distinct from the four standard nucleotides (deoxyadenosine triphosphate (dATP), deoxythymidine triphosphate (dTTP), deoxyguanosine triphosphate (dGTP), and deoxycytidine triphosphate (dCTP), and is capable of being incorporated into new strands; and
(b) performing one pass extension or cycles of extension reactions of the first primer on target nucleic acid template to produce copy of modified complementary strands,

The method may further comprise:
(c) adding a second polymerase which is capable of using the modified complementary strand as template; and
(d) replicating or amplifying the modified complementary strands using the second polymerase. In this step, the original strands may also be replicated or amplified.

The cycles of extension reactions of step (b) may comprise at least one cycle (one pass extension), preferably 2 to 50 cycles, or more preferably 2 to 40 cycles.

The step (c) may comprise additionally adding second primer which is capable to be extended in step (d).

In one embodiment, a second polymerase which is capable of using the modified complementary strand as template may be used to replicate the modified complementary strands in the presence of a second unusual nucleotide generating a modified copy of the modified complementary strand, wherein the second polymerase cannot or is incapable of efficiently making further copies using the modified copy as template.

Such a method further comprises
(e) adding a third polymerase which is capable of using the modified copy of the modified complementary strand as a template; and
(f) replicating the modified copy and/or the modified complementary strands using the third polymerase.

Optionally after step (b) the method further comprises removing some or all of the nucleoside triphosphate(s) and/or primers by purification and/or an enzymatic reaction.

Preferably the unusual nucleoside triphosphate may be deoxyuridine triphosphate (dUTP), or 5-Methyl-2′-deoxycytidine-5′-Triphosphate. Any nucleotide chemically or functionally distinct from the four standard nucleotides (deoxyadenosine triphosphate (dATP), deoxythymidine triphosphate (dTTP), deoxyguanosine triphosphate (dGTP), and deoxycytidine triphosphate (dCTP)) is termed “unusual nucleotide”. The unusual nucleoside triphosphate may be selected from: ribonucleoside triphosphate, deoxyinosine triphosphate, 2′-O-Methyladenosine-5′-Triphosphate, 2′-O-Methylcytidine-5′-Triphosphate, 2′-O-Methylguanosine-5′-Triphosphate, 2′-O-Methyluridine-5′-Triphosphate, 2′-Deoxyuridine-5′-Triphosphate or 5-Methyl-2′-deoxycytidine-5′-Triphosphate.

In one embodiment, the unusual nucleotide is 5-Methyl-2′-deoxycytidine-5′-Triphosphate, wherein after step (b) the DNA mixture is deaminated by either chemical and/or enzymatic processes. The modified complementary strands are protected from deamination, the original strands are deaminated on the sites not methylated. The deamination may be a chemical conversion by bisulphate. The modified complementary strands and/or the deaminated original strands or copies of the deaminated original strands are amplified in step (d). In one embodiment, after deamination and before step (b) the deaminated original strands may be linearly amplified with or without unusual nucleotide to produce copies of the deaminated original strands.

The polymerase may be a DNA polymerase. The first DNA polymerase may be an archaeal DNA polymerase, or a modified archaeal DNA polymerase. The archaeal DNA polymerase, or modified archaeal DNA polymerase or Family B polymerase may be Pfu DNA polymerase, Phusion DNA polymerase, Vent DNA polymerase, KOD DNA polymerase, Vent (exo-) DNA polymerase, Deep Vent (exo-) DNA polymerase, Deep Vent DNA polymerase, Q5, therminator DNA polymerase or any combination thereof. The second DNA polymerase may be the same polymerase as the first polymerase, as long as the step (d) reaction can be carried efficiently. After optional removal of the unusual nucleotide after linear amplification, any polymerase which using the standard nucleotide is capable of efficiently extending (replicating) can be used as second polymerase. Alternatively, a polymerase capable of replicate the modified complementary strand even in the presence of unusual nucleotide can be used as second polymerase.

The wordings “cannot efficiently copy” or “incapable of efficiently making” mean that compared to the standard condition of replication or amplification, in the presence of unusual nucleotides or under other conditions a group of polymerases may have less than 100% efficiency to replicate such as 99% efficiency, or 95% efficiency, or 90% efficiency, or 80% efficiency, or 70% efficiency, or 60% efficiency, or 50% efficiency, or 40% efficiency, or 30% efficiency, or 20% efficiency, or 10% efficiency, or 5% efficiency. Sometimes one may not know at what efficiency a polymerase replicate or amplify a nucleic acid, as long as a polymerase capable of one pass extension or linearly amplification but performing suboptimally in PCR amplification can be used as first polymerase.

The first primer may be a set of random or degenerate primers which comprise 3′ random or degenerate sequence with or without 5′ universal tail sequence, wherein the primers are capable of hybridising to any random region, wherein the presence of the unusual nucleoside triphosphate in the extension products results in the extension products directly or indirectly not being efficiently used as templates for the first DNA polymerase to replicate the modified complementary strand.

The random or degenerate regions may be 3, 4, 5, 6, 7, 8, 9, 10, or between 11-20, 21-30, or more than 31 base pairs in length, preferably between 6 and 10 bp in length. The random primers may be all deoxyribose nucleic acids, ribose nucleic acids, unusual nucleotides, or any combination in any combination thereof.

The first primer may be a set of multiple target specific primers. The primer sequence may comprise the 3′ target specific sequence with or without 5′ universal tail sequence, wherein the primers are capable of annealing to first strand or/and complementary second strand of target regions, wherein in the presence of the unusual nucleoside triphosphate the extension products cannot be efficiently used as templates for the first DNA polymerase to replicate the modified complementary strand.

The primers may comprise a 3′ target specific sequence, an optional central series of nucleotides which is capable of acting as a unique molecular identifier, and a 5′ universal tail sequence, wherein the unique molecular identifier is of a suitable length and comprises a mixture of random nucleotides or degenerated nucleotides which acts as a unique molecular identifier (UMI) or molecular barcode, allowing for the identification of PCR duplicates in massively parallel sequencing data.

The 5′ universal tails may comprise at least two different sequences for the opposing primers which flank a desired length of region to be amplified, wherein the two opposing primers in proximity which flank an undesired length of region have the same universal tail sequence. The universal tail of primers may be a single population of sequences. It may be a population of 2, 3, 4, 5, 6, 7, 8, 9, 10, between 11-20, 21-30, 31-40, 41-50, 51-100, or more than 100 different universal sequences. When using more than one universal tail it is expected that head-to-head primers will have the same sequence.

The primers in the first set may comprise the same sequence of 5′ universal tails and as such are able to act as universal primers. The second set of primers may comprise universal primers or/and target specific primers, wherein the universal primers comprise sequence identical or substantially identical to the 5′ tail sequences of the primers of the first set, wherein the target specific primers comprise 3′ target specific sequence and 5′ universal tails with or without a central region capable of acting as a UMI.

The first primers may be universal primers. The target polynucleotides of interest may be ligated to adaptors, or may be extended by ATOM-seq method. The first primers may comprise universal primers which sequence is complementary or substantially complementary to the adaptor sequence or universal sequence of the ATO of ATOM-seq extension products.

The present invention further provides a method of preparing a sequencing library, the method comprising:
(a) providing a reaction mixture(s), each reaction mixture comprising nucleic acids to be sequenced or targeted, a first polymerase which may be a DNA polymerase, unusual nucleoside triphosphates, additional standard nucleotides as necessary, and a first set of primers, wherein the polymerase is capable of extending primers using the target nucleic acids as templates and incorporating the unusual nucleotide into extension products which are modified complementary strands, and is incapable of efficiently making a copy using the modified complementary strand as template, wherein the unusual nucleoside triphosphate is distinct from the four standard nucleotides: deoxyadenosine triphosphate (dATP), deoxythymidine triphosphate (dTTP), deoxyguanosine triphosphate (dGTP), or deoxycytidine triphosphate (dCTP), and is capable of being incorporated into new strands, wherein the first set of primers comprise target specific primers, universal primers or random primers;
(b) performing extension reaction of primer and target nucleic acid template to produce modified complementary strands under extension condition, wherein the extension condition comprises buffer, any of four standard nucleoside triphosphates and appropriate temperature;
(c) optionally removing the nucleoside triphosphate and/or primers by purification and/or an enzymatic reaction;
(d) performing amplification of the modified complementary strands and/or original strands using a second set of primers and using a second DNA polymerase which is preferably capable of using the modified complementary strand as template, wherein the amplification can be linear amplification or PCR amplification; and
(e) processing the products of step (d) to complete the library preparation for massive parallel sequencing which may involve a third set of primers which are universal primers and allow for incorporation of sample indexes.

In one embodiment for methylation analysis after step (b) the DNA mixture may be deaminated by either chemical and/or enzymatic processes.

The step (b) may be a linear amplification by performing the extension once or at least twice to produce multicopy of modified complementary strands.

The present invention provides another method of preparing a sequencing library for methylation analysis comprising:
(a) providing a reaction mixture(s), each reaction mixture comprising nucleic acids to be sequenced, a first DNA polymerase, unusual nucleoside triphosphates and a first set of primers, wherein the unusual nucleoside triphosphates may be 5-Methyl-2′-deoxycytidine-5′-Triphosphate or any other unusual nucleotide, wherein the polymerase is capable of extending primers using the target nucleic acids as templates and incorporating the unusual nucleotide into extension products which are modified complementary strands, wherein the first set of primers comprise target specific primers, universal primers or random primers;
(b) performing extension reaction of primer on target nucleic acid template to produce modified complementary strands under extension condition, wherein the extension condition comprises buffer, any of four standard nucleoside triphosphates and appropriate temperature;
(c) deaminating the DNA mixture by either chemical and/or enzymatic processes; (d) purifying the DNA mixture;
(e) performing amplification of the DNA mixture using a second set of primers and using a second DNA polymerase, wherein the DNA mixture may comprise modified complementary strands, deaminated original strands, or copies of deaminated original strands, wherein the amplification may be linear amplification or PCR amplification which comprises amplification of modified complementary strands and/or amplification of deaminated original strands or copies of deaminated original strand; and
(f) processing the products of step (e) to complete the library preparation for massive parallel sequencing.

The step (b) may be a linear amplification by performing the extension at least twice to produce multicopy of modified complementary strands.

The deamination may be a chemical conversion by bisulphate. After deamination the deaminated original strands may be linearly amplified with or without unusual nucleotides before step (e) to produce copies of deaminated original strands.

The modified complementary strands with incorporated 5-Methyl-2′-deoxycytidine are protected from deamination, whereby the modified complementary strands keep the original DNA information, which can be used for mutation analysis. The deaminated original strand can be used for methylation detection. In this method, the mutation detection and methylation detection can be performed in the same reactions wherein the PCR amplification of mutation sites and methylation sites can be performed in the same tube. Alternatively, the PCR amplification of mutation sites and methylation sites can be performed in different tubes.

The present invention also provide a kit for performing a method according to any preceding embodiment comprising: (a) a first DNA polymerase (b) one or more standard nucleotides: deoxyadenosine triphosphate (dATP), deoxythymidine triphosphate (dTTP), deoxyguanosine triphosphate (dGTP), and deoxycytidine triphosphate (dCTP), (c) deoxyuridine triphosphate (dUTP) or 5-Methyl-2′-deoxycytidine-5′-Triphosphate, (d) two or more primers, and (e) a second polymerase which may be a DNA polymerase.

Described herein is a method of processing target nucleic acids, wherein a target nucleic acid is either:
(i) a double-stranded duplex which comprises a first strand and a complementary second strand; or
(ii) a single-stranded molecule which is a first strand or its complementary second strand
   wherein the method comprises:
(a) providing a reaction mixture(s), each reaction mixture comprising a first DNA polymerase, unusual nucleoside triphosphates and a first set of primers, wherein the polymerase is capable of extending primers using the target nucleic acids as templates and incorporating the unusual nucleotide into extension products which are modified complementary strands, and is incapable of efficiently making a copy using the modified complementary strand as template for extension of primer in the opposite orientation, wherein the unusual nucleoside triphosphate is distinct from the four standard nucleotides: deoxyadenosine triphosphate (dATP), deoxythymidine triphosphate (dTTP), deoxyguanosine triphosphate (dGTP), or deoxycytidine triphosphate (dCTP), and is capable of being incorporated into new strands but cannot being copied as template by said first DNA polymerase; and
(b) performing an extension reaction of primer and target nucleic acid template to produce modified complementary strands under extension conditions, wherein the extension condition comprises buffer, unusual nucleoside triphosphates, any of standard nucleoside triphosphates and appropriate temperature.

The method may further comprise
(c) adding a second DNA polymerase which is capable of using the modified complementary strand as template; and
(d) replicating the modified complementary strands using the second DNA polymerase.

Step (b) may be a linear amplification by performing the extension at least twice to produce multicopy of single-stranded modified complementary strands, preferably more than twice.

The unusual nucleoside triphosphate may be deoxyuridine triphosphate (dUTP).

The unusual nucleoside triphosphate may be selected from a group of modified or naturally occurring nucleotides, including but is not limited to: ribonucleoside triphosphate, deoxyinosine triphosphate, 2′-O-Methyladenosine-5′-Triphosphate, 2′-O-Methylcytidine-5′-Triphosphate, 2′-O-Methylguanosine-5′-Triphosphate, 2′-O-Methyluridine-5′-Triphosphate, 2′-fluoro-NTPs (Kasuya et al., 2014), glyceronucleotides (gNTPs) (Chen et al., 2009), 7′,5′-Bicyclo-NTPs (Diafa et al., 2017), 3-phosphono-L-Ala-dNMPs (Yang and Herdewijn, 2011; Giraut et al., 2012), 3′-2′-phosphonomethyl-threosyl-NTPs (Renders et al., 2007, 2008), 5′-3′-phosphonomethyl-dNTPs (Renders et al., 2007, 2008), 2′-deoxy-2′-isonucleoside (iNTPs) (Ogino et al., 2010), 3′-deoxyapionucleotide 3′-triphosphates (apioNTPs) (Kataoka et al., 2008, 2011), 5-trifluoromethyl-dUTP (Holzberger and Marx, 2009) and 4′-C-aminomethyl-2′-O-methyl-TTP (Nawale et al., 2012), amphiphilic dNTP analogues, and Locked nucleic acid (LNAs) nucleotides.

The first polymerase may be a DNA polymerase which may be any DNA polymerase which is capable of generating a copy of a target nucleic acid in a primer independent manor, or, a primer dependent manor by extending primers using the target nucleic acids as templates and incorporating the unusual nucleotide into extension products which are modified complementary strands, and is incapable of efficiently making a copy using the modified complementary strand as template for extension of primer in the opposite orientation.

Preferably, the first polymerase is archaeal DNA polymerase, or modified archaeal DNA polymerase whose modification may be a naturally occurring variant or a derivate polymerase generated by selected or targeted or random mutagenesis or evolution.

The archaeal DNA polymerase, or modified archaeal DNA polymerase or Family B polymerase may be selected from group but is not limited to; Pfu DNA polymerase, Phusion DNA polymerase, Vent DNA polymerase, KOD DNA polymerase, Vent (exo-) DNA polymerase, Deep Vent (exo-) DNA polymerase, Deep Vent DNA polymerase, Q5, “therminator DNA polymerase”, any derivate(s) thereof, or, any combination thereof.

The first polymerase may be an RNA polymerase of reverse transcriptase or other system which has been selectively or randomly engineered to be capable of functioning equivalently to a DNA polymerase whereby it can produce copies of a nucleic acid template by a process of amplification.

The first set of primers may be a plurality of primers which comprise combinations of random nucleotides to generate a random primer. The random primer may be used to non-specifically globally amplify whole nucleic acids in a sample.

The first set of primers may be target specific primers, and/or universal primers.

The first set of primers may be a mixture of multiple primers, comprising primers capable of annealing to first strand or second strand of a target regions to be amplified, wherein in the presence of the unusual nucleoside triphosphate the extension products cannot be used as templates thus reducing the chance of non-specific and or unwanted PCR amplification products.

The primers may themselves contain unusual nucleotides to prevent themselves from being copied in the first reaction the resultant amplification products would be incomplete copies.

The first set of primers may be a mixture of multiple primers, comprising primers capable of annealing to first strand and second strand of a target region to be amplified, wherein in the presence of the unusual nucleoside triphosphate the opposing primers which form a pair of primers are only capable of linear amplifications as the amplification products themselves cannot efficiently be used as templates.

The primers may comprise a 3′ target specific sequence, an optional central series of nucleotides which is capable of acting as a unique molecular identifier (UMI), and a 5′ universal tail sequence, wherein the unique molecular identifier is of a suitable length and comprises a mixture of random nucleotides, degenerated nucleotides which allow for the identification of PCR duplicates in massively parallel sequencing.

The UMI may be 1, 2, 3, 4, 5, 6, 7, 8, 9, 10, 11, 12, 13, 14, 15, 16, 17, 18, 19, or 20 or more base pairs in length, preferentially the UMI would be 6 to 16 bp in length.

The 5′ universal tails may comprise of the same sequence, or at least two different sequences from a pool of at least 2, 3, 4, 5, 6, 7, 8, 9, or 10 or more sequences, wherein the two opposing primers in proximity have the same universal tail sequence. The primers in the first set may comprise the same sequence of the first 5′ universal tails. The target specific primers in the second set in the PCR reaction may comprise the second 5′ universal tails, which is different from the first 5′ universal tails of the primers of the first set. In a linear amplification, in heavily tiled region head-to-head linear primers comprising the same first 5′ universal tail sequence and the use of an unusual nucleotide have a synergistic effect in reducing nonspecific PCR products while also allowing for fully tiled linear amplification of the target genomic regions. In the followed PCR, by using head-to-head PCR primers which comprise the second 5′ universal tail sequence in combination of universal primer with first tail sequence of linear primer, we are able to generate overlapping tiled amplicons allowing for easy whole gene coverage where each molecule contains a UMI to help improve the accuracy of mutation detection by allowing for error correction of PCR artefacts. The first 5′ universal tail sequence is different from the second 5′ universal tail sequence. The original strand information is NOT lost in products, when looking for mutations, any mutations found can be attributed to sense or antisense strands The primers may comprise a 3′ target specific sequence, and an affinity label either at the primers 5′ end or in between the 3′ and 5′ ends, wherein the affinity label may be a biotin.

The method optionally further comprises a step of removing the unusual nucleoside triphosphate and/or primers by purification or an enzymatic reaction. The purification may use avidin solid supports.

The enzymatic reaction may be a dephosphorylation reaction, which uses a phosphatase, which may include but is not limited to Antarctic Phosphatase, Quick CIP, Shrimp Alkaline Phosphatase (rSAP).

The method may further comprise a step of amplification of the modified complementary strands using a second set of primers and using a second DNA polymerase which is capable of using the modified complementary strand as template, wherein the second DNA polymerase may be added after the step of removing the unusual nucleoside triphosphate, or directly after the step (b).

In another embodiment of the invention, in the step (c) without adding target specific second primers, the second DNA polymerase may extend the hybridised first primers or partially extended first primers of step (b) on the template of the modified complementary strands to make a full complementary copy of the modified complementary strands. After this, the universal second primer may be used to amplify the modified complementary strands. The universal second primer has the sequence substantially identical to the 5′ tail sequence of the first primer. The second DNA polymerase may be added after purifying the product of step (b), or directly after the step (b).

The second set of primers may comprise universal primers or/and target specific primers, wherein the universal primers comprise sequence identical or substantially identical to the 5′ tail sequences of the primers of first set.

Disclosed is a method of preparing a sequencing library, comprising:
(a) providing a reaction mixture(s), each reaction mixture comprising target nucleic acids to be sequenced, a first DNA polymerase, unusual nucleoside triphosphates and a first set of primers, wherein the polymerase is capable of extending primers using the target nucleic acids as templates and incorporating the unusual nucleotide into extension products which are modified complementary strands, and can make further copies of any templates or preferably is incapable of efficiently making a copy using the modified complementary strand as template for extension of primer in the opposite orientation, wherein the unusual nucleoside triphosphate is distinct from the four standard nucleotides: deoxyadenosine triphosphate (dATP), deoxythymidine triphosphate (dTTP), deoxyguanosine triphosphate (dGTP), or deoxycytidine triphosphate (dCTP), and is capable of being incorporated into new strands but cannot being copied as template by said first DNA polymerase, wherein the first set of primers comprise target specific primers, universal primers or random primers;
(b) performing extension reaction of primer and target nucleic acid template to produce modified complementary strands under extension condition, wherein the extension condition comprises buffer, any of usual nucleoside triphosphates and appropriate temperature;
(c) (optional) removing the unusual nucleoside triphosphate and/or primers by purification or an enzymatic reaction;
(d) performing amplification of the modified complementary strands using a second set of primers and using a second DNA polymerase which is capable of using the modified complementary strand as template, wherein the amplification can be linear amplification or PCR amplification
(e) processing the products of step (d) to complete the library preparation for massive parallel sequencing

In the method step (b) may be a linear amplification by performing the extension at least twice to produce multicopy of single-stranded modified complementary strands. The cycles of linear amplification may be 2 to 40 cycles. Alternatively, the step (b) may be one pass of extension.

Disclosed is a kit for performing a method according to any preceding claim or method comprising at least but not limited to:
a. Reaction mixes including all necessary reagents for amplification of target polynucleotides.
b. All necessary unusual nucleoside triphosphate(s) either in separate tubes or contain premixed within a master mix.
c. One or more pools of amplification primers, either
a first set of multiple target specific primers or random primers as defined in any previous embodiments which are forward and/or reverse primers capable of annealing to multiple target sequences of either a first strand or a second strand, or both strands of the target sequences; and/or
a second set of multiple target specific primers or/and universal primers as defined in any of previous embodiments; and/or
primers for generating double-stranded PCR products suitable for massively parallel sequencing.

A sample may contain RNA to be analysed. The RNA may be converted to single stranded cDNA as target nucleic acids. Any method of converting RNA to cDNA can be used. For example, a random hexamer or target specific primers can be used to prime cDNA syntheses. The RNA can also be converted into double stranded cDNA as target nucleic acids. In one embodiment, the single stranded cDNA (ss cDNA) is generated by random hexamer or a like in the presence of a reverse transcriptase. After ss cDNA is synthesised, the reaction may be purified before processing to step (a). In another simple embodiment, the ss cDNA reaction is not purified, but is directly processed to step (a).

Disclosed is a method of preparing a sequencing library, comprising:
(a) providing a reaction mixture(s), each reaction mixture comprising target nucleic acids to be sequenced, a first DNA polymerase, unusual nucleoside triphosphates and a first set of primers, wherein the polymerase is capable of extending primers using the target nucleic acids as templates and incorporating the unusual nucleotide into extension products which are modified complementary strands, and can make further copies of any templates or preferably is incapable of efficiently making a copy using the modified complementary strand as template for extension of primer in the opposite orientation, wherein the unusual nucleoside triphosphate is distinct from the four standard nucleotides: deoxyadenosine triphosphate (dATP), deoxythymidine triphosphate (dTTP), deoxyguanosine triphosphate (dGTP), or deoxycytidine triphosphate (dCTP), and is capable of being incorporated into new strands but cannot or cannot efficiently be copied as template by said first DNA polymerase, wherein the first set of primers comprise target specific primers or random primers;
(b) performing extension amplification reactions of primer and target nucleic acid template to produce modified complementary strands under extension condition, wherein the extension condition comprises buffer, any standard and or usual nucleoside triphosphates and appropriate temperature;
(c) (optional) removing the unusual nucleoside triphosphate and/or primers by purification or an enzymatic reaction;
(d) performing a second amplification of the modified complementary strands using a second set of primers and using a second polymerase which is capable of using the modified complementary strand as template with a second unusual nucleotide wherein the unusual nucleoside triphosphate is distinct from the four standard nucleotides: deoxyadenosine triphosphate (dATP), deoxythymidine triphosphate (dTTP), deoxyguanosine triphosphate (dGTP), or deoxycytidine triphosphate (dCTP), and is capable of being incorporated into new modified copies of modified complementary strands but cannot or cannot efficiently be copied as template by said second polymerase
(e) performing a third amplification using a third set of primers and a third polymerase which is capable of using the modified copies of modified complementary strands as a template to generate PCR copies
(e) processing the PCR products of step (e) to complete the library preparation for massive parallel sequencing

In the method step (b) and/or step (d) may be a linear amplification by performing the extension at least twice to produce multicopy of single-stranded modified complementary strands. The cycles of linear amplification may be 2 to 100 cycles or preferably 2 to 40 cycles. Alternatively, the step (b) may be one pass of extension.

In one aspect, the invention provides methods of processing target nucleic acids from one or more samples, wherein a target nucleic acid in a sample may be a single-stranded molecule (which is referred to as the sense or first strand, wherein its complement is referred to as the antisense or second strand) or double-stranded duplex which comprises a duplex between a first and a complementary second strand, wherein the method comprises:
(a) providing a reaction mixture(s), each reaction mixture comprising a first DNA polymerase, unusual nucleoside triphosphates and a first set of primers, wherein the polymerase is capable of extending primers using the target nucleic acids as templates and incorporating the unusual nucleotide into extension products which are now modified complementary strands, and can make further copies of any templates or preferably is incapable of efficiently making further copies using the modified complementary strand as template for extension of primers in the opposite orientation, wherein the unusual nucleoside triphosphate is distinct from the four standard nucleotides which may or may not be present in the reaction mixture: deoxyadenosine triphosphate (dATP), deoxythymidine triphosphate (dTTP), deoxyguanosine triphosphate (dGTP), or deoxycytidine triphosphate (dCTP), and is capable of being incorporated into new strands but polynucleotides containing this unusual nucleotide cannot be copied as template by said first DNA polymerase. In one embodiment the first set of primers comprise target specific primers (FIG. 1), in another embodiment, the first set of primers comprise random primers, which are used for amplification of all nucleic acids in a reaction, in a further embodiment, the first set of primers comprise universal primers which are capable of annealing to the universal adaptor or ATO sequence
(b) performing an extension reaction of primer and target nucleic acid template to produce modified complementary strands under extension conditions, wherein the extension condition comprises buffer, any of standard nucleotides, usual nucleoside triphosphates and appropriate temperature(s).

The method may further comprise optional steps (c) where the unused nucleotides or/and unused primers are removed, made inert, or made otherwise non-functional which therefore allows for the modified complementary strands to be used as a template in subsequent downstream processes; (d) if not accomplished as part of step (c) (optional) treating the products step (b) to enrich the products; (e) additional rounds of extension reactions which may be one or more rounds of linear or PCR amplification of the products of step (b) using primers to generate double-stranded products, wherein the product of this step may be used directly or indirectly for sequencing.

The method may further comprise step (f) processing the PCR products of step (e) to complete the sequencing library preparation for massive parallel sequencing such as a NGS platform.

The step (c) and/or step (f) may comprise removing the unreacted primers, wherein the removing of the unreacted primers may comprise purifying the single-stranded linear amplification products of step (c) or double-stranded product of step (f), for example a bead or column-based method is used to remove unreacted primers. The removing of the unreacted primers may comprise treating the amplification products by enzymatic digestion to remove the unreacted primers, wherein the enzymatic digestion may be exonuclease I digestion.

The second set of primers may be a set of target specific primers or universal primers having the sequence substantially identical to the tail sequence of the first primers, or both a set of target specific primers and universal primer. After the step (b) the method may comprise hybridising the single-stranded modified complementary strands to a second set of target-specific primers. The hybridised target-specific primers of the second set of primers may be extended on the single-stranded modified complementary strands with a single round of extension, one pass extension, or multiple rounds of linear amplification. In another embodiment of the invention, without adding a second set of target specific primers or random primers, the second DNA polymerase may extend the hybridised first primers or partially extended first primers of step (b) on the template of the modified complementary strands to make a full complementary copy of the modified complementary strands. The resulting double stranded modified complementary strand may be used for a subsequent amplification using universal second primers. Generation of double stranded modified complementary strand and subsequent amplification may be performed in a single reaction, in which the second primer may be the solely universal primer without needing target specific primer. Optionally, any target-specific or universal primer may comprise an affinity label or 5′ universal tail portion, wherein the 5′ universal tail portion of the hybridised target-specific primers are hybridised with an affinity-labelled oligonucleotide complementary to the 5′ universal tail. The affinity label may be biotin, the complex of the hybridised amplification products/target-specific oligonucleotides/biotin-labelled oligonucleotide are captured by avidin solid supports.

The target specific primer may comprise a 5′ tail portion and a 3′ target complementary portion (FIG. 1b). The 5′ tail portion or an additional portion not complementary to the target sequence may comprise a unique molecular identifier (UMI), or/and sequence(s) compatible for a NGS platform, which may comprise universal PCR primer sequence, NGS sequencing primer sequence, and/or NGS adaptor sequences.

In the step (a), first set of target-specific primer(s) are present in a reaction, wherein the target-specific primer(s) in the first set is capable of hybridising to the first strand, the second strand, or both first and second stands of a target duplex.

During traditional PCR one or more primers form pairs of opposing forward and reverse primers which are used to generate an exponential amplification of the region of the target polynucleotide between any two opposing primers. This invention describes a method for promoting two opposing primers which contain UMIs (also known as barcodes) to only perform linear amplifications, in a single tube. This is termed “barcoded opposing strand orientated” linear amplification. During these linear amplifications the newly generated amplification product is incapable or must have a significantly reduced efficiency for acting as a template in all subsequent cycles of amplifications after the one in which is it created. This may be accomplished by the addition of an “unusual nucleotide” which acts to render the primer extension amplification product non-copyable by the enzyme which made it, the product is a modified complementary strand. In step (b) therefore linear amplification can be performed with opposing or non-opposing primers. This is a process which is impossible with traditional PCR in a single tube and is only possible when the starting template is divided into two samples.

In an embodiment of the invention the target polynucleotide may undergo a chemical and/or enzymatic and/or equivalent conversion reaction to convert cytosine nucleotides which do or do not have ‘epigenetic marks’ to uracil or a derivative or equivalent to uracil prior to use in an implementation of the invention. The target polynucleotide may contain epigenetic mark(s) which may be comprised of one or more or combination of 5-methylcytosine (5mC), 5-hydroxymethylcytosine (5hmC), 5-formylcytosine (5fC) or 5-carboxycytosine (5caC).

In another embodiment the target polynucleotide may be linearly amplified by a first polymerase with primer(s) and unusual nucleotide(s) which may include but are not limited to 5-Methyl-2′-deoxycytidine-5′-Triphosphate, 5-hydroxyMethyl-2′-deoxycytidine-5′-Triphosphate, 5-formyl-2′-deoxycytidine-5′-Triphosphate or 5-Carboxy-2′-deoxycytidine-5′-Triphosphate or any combination thereof which may completely or partially replace dCTP to produce a modified first complementary strand where cytosines have been replaced with a modified version of cytosine which are resistant to subsequent modification. The original target polynucleotide and modified complementary strand undergo a chemical and/or enzymatic and/or equivalent conversion reaction to convert cytosine nucleotides which do or do not have ‘epigenetic marks’ to uracil or a derivative or equivalent to uracil producing deaminated original strands. The deaminated original target polynucleotide and protected modified complementary strands may then be used for subsequent amplification reactions. These amplification reactions may use a second set of primers which are designed to only amplify the protected modified complementary strand allowing for high sensitivity detection of mutations. These amplification reactions may use a second set of primers which are designed to only amplify the deaminated original target polynucleotide allowing for high sensitivity detection of epigenetic signals. These amplification reactions may use a second set of primers which are designed to amplify both the deaminated original target polynucleotide and protected modified complementary strands allowing for targeted enrichment of both mutations and epigenetic signals. The amplification reactions designed to amplify both the deaminated original target polynucleotide and protected modified complementary strands may be in the same reaction vessel or the sample may be divided into two reactions where each enriches one of the two populations of polynucleotides.

In an embodiment of the invention the unusual nucleotide is 2′-Deoxyuridine-5′-Triphosphate (dUTP). The dUTP may be used to completely replace dTTP, or, may be used in combination with dTTP in the presence of none, one of, two or, or all dATP, dCTP and dGTP. The dUTP may be used in the absence of dTTP (a ratio of 1:0), it may be used at a ratio of 100:1, 50:1, 25:1, 10:1, 5:1, 1:1, 1:5 or at higher or lower ratios as long as the polymerase used is sufficiently inhibited from using the unusual nucleotide containing modified complementary strands to prevent PCR from occurring.

In another embodiment the unusual nucleotide can be any nucleotide capable of being incorporated during primer extension which prevents the product from efficiently being used as a template and may be chosen from the following non exhaustive list; ribonucleoside triphosphate, deoxyinosine triphosphate, 2′,3′-Dideoxyadenosine-5′-O-(1-Thiotriphosphate), 2′,3′-Dideoxyadenosine-5′-Triphosphate, 2′,3′-Dideoxycytidine-5′-O-(1-Thiotriphosphate), 2′,3′-Dideoxycytidine-5′-Triphosphate, 2′,3′-Dideoxyguanosine-5′-O-(1-Thiotriphosphate), 2′,3′-Dideoxyguanosine-5′-Triphosphate, 2′,3′-Dideoxyinosine-5′-Triphosphate, 2′,3′-Dideoxythymidine-5′-Triphosphate, 2′,3′-Dideoxyuridine-5′-O-(1-Thiotriphosphate), 2′,3′-Dideoxyuridine-5′-Triphosphate, 2′-Amino-2′-deoxyadenosine-5′-Triphosphate, 2-Amino-2′-deoxyadenosine-5′-Triphosphate, 2′-Amino-2′-deoxycytidine-5′-Triphosphate, 2′-Amino-2′-deoxyuridine-5′-Triphosphate, 2-Amino-6-chloropurineriboside-5′-Triphosphate, 2-Amino-6-Cl-purine-2′-deoxyriboside-Triphosphate, 2-Aminoadenosine-5′-Triphosphate, 2-Aminopurine-2′-deoxyriboside-Triphosphate, 2-Aminopurine-riboside-5′-Triphosphate, 2′-Azido-2′-deoxyadenosine-5′-Triphosphate, 2′-Azido-2′-deoxycytidine-5′-Triphosphate, 2′-Azido-2′-deoxyguanosine-5′-Triphosphate, 2′-Azido-2′-deoxyuridine-5′-Triphosphate, 2′-Deoxyadenosine-5′-O-(1-Boranotriphosphate), 2′-Deoxyadenosine-5′-O-(1-Thiotriphosphate), 2′-Deoxyadenosine-5′-Triphosphate, 2′-Deoxycytidine-5′-O-(1-Boranotriphosphate), 2′-Deoxycytidine-5′-O-(1-Thiotriphosphate), 2′-Deoxycytidine-5′-Triphosphate, 2′-Deoxyguanosine-5′-O-(1-Boranotriphosphate), 2′-Deoxyguanosine-5′-O-(1-Thiotriphosphate), 2′-Deoxyguanosine-5′-Triphosphate, 2′-Deoxyinosine-5′-Triphosphate, 2′-Deoxynucleoside-5′-Triphosphate Set, 2′-Deoxy-P-nucleoside-5′-Triphosphate, 2′-Deoxythymidine-5′-O-(1-Boranotriphosphate), 2′-Deoxythymidine-5′-O-(1-Thiotriphosphate), 2′-Deoxythymidine-5′-Triphosphate, 2′-Deoxyuridine-5′-Triphosphate, 2′-Deoxyzebularine-5′-Triphosphate, 2′-Fluoro-2′-deoxyadenosine-5′-Triphosphate, 2′-Fluoro-2′-deoxycytidine-5′-Triphosphate, 2′-Fluoro-2′-deoxyguanosine-5′-Triphosphate, 2′-Fluoro-2′-deoxyuridine-5′-Triphosphate, 2′-Fluoro-thymidine-5′-Triphosphate, 2′-O-Methyl-2-aminoadenosine-5′-Triphosphate, 2′-O-Methyl-5-methyluridine-5′-Triphosphate, 2′-O-Methyladenosine-5′-Triphosphate, 2′-O-Methylcytidine-5′-Triphosphate, 2′-O-Methylguanosine-5′-Triphosphate, 2′-O-Methylinosine-5′-Triphosphate, 2′-O-Methyl-N6-Methyladenosine-5′-Triphosphate, 2′-O-Methylpseudouridine-5′-Triphosphate, 2′-O-Methyluridine-5′-Triphosphate, 2-Thio-2′-deoxycytidine-5′-Triphosphate, 2-Thiocytidine-5′-Triphosphate, 2-Thiothymidine-5′-Triphosphate, 2-Thiouridine-5′-Triphosphate, 3′-Amino-2′,3′-dideoxyadenosine-5′-Triphosphate, 3′-Amino-2′,3′-dideoxycytidine-5′-Triphosphate, 3′-Amino-2′,3′-dideoxyguanosine-5′-Triphosphate, 3′-Amino-2′,3′-dideoxythymidine-5′-Triphosphate, 3′-Azido-2′,3′-dideoxyadenosine-5′-Triphosphate, 3′-Azido-2′,3′-dideoxycytidine-5′-Triphosphate, 3′-Azido-2′,3′-dideoxyguanosine-5′-Triphosphate, 3′-Azido-2′,3′-dideoxythymidine-5′-O-(1-Thiotriphosphate), 3′-Azido-2′,3′-dideoxythymidine-5′-Triphosphate, 3′-Azido-2′,3′-dideoxyuridine-5′-Triphosphate, 3′-Deoxy-5-Methyluridine-5′-Triphosphate, 3′-Deoxyadenosine-5′-Triphosphate, 3′-Deoxycytidine-5′-Triphosphate, 3′-Deoxyguanosine-5′-Triphosphate, 3′-Deoxythymidine-5′-O-(1-Thiotriphosphate), 3′-Deoxyuridine-5′-Triphosphate, 3′-O-(2-nitrobenzyl)-2′-Deoxyadenosine-5′-Triphosphate, 3′-0-(2-nitrobenzyl)-2′-Deoxyinosine-5′-Triphosphate, 3′-O-Methyladenosine-5′-Triphosphate, 3′-O-Methylcytidine-5′-Triphosphate, 3′-O-Methylguanosine-5′-Triphosphate, 3′-O-Methyluridine-5′-Triphosphate, 4-Thiothymidine-5′-Triphosphate, 4-Thiouridine-5′-Triphosphate, 5,6-Dihydro-5-Methyluridine-5′-Triphosphate, 5,6-Dihydrouridine-5′-Triphosphate, 5-[(3-Indolyl)propionamide-N-allyl]-2′-deoxyuridine-5′-Triphosphate, 5-Aminoallyl-2′-deoxycytidine-5′-Triphosphate, 5-Aminoallyl-2′-deoxyuridine-5′-Triphosphate, 5-Aminoallylcytidine-5′-Triphosphate, 5-Aminoallyluridine-5′-Triphosphate, 5′-Amino-G-Monophosphate, 5′-Biotin-A-Monophosphate, 5′-Biotin-dA-Monophosphate, 5′-Biotin-dG-Monophosphate, 5′-Biotin-G-Monophosphate, 5-Bromo-2′,3′-dideoxyuridine-5′-Triphosphate, 5-Bromo-2′-deoxycytidine-5′-Triphosphate, 5-Bromo-2′-deoxyuridine-5′-Triphosphate, 5-Bromocytidine-5′-Triphosphate, 5-Bromouridine-5′-Triphosphate, 5-Carboxy-2′-deoxyuridine-5′-Triphosphate, 5-Carboxy-2′-deoxycytidine-5′-Triphosphate, 5-Carboxycytidine-5′-Triphosphate, 5-Carboxymethylesteruridine-5′-Triphosphate, 5-Carboxyuridine-5′-Triphosphate, 5-Fluoro-2′-deoxyuridine-5′-Triphosphate, 5-Formyl-2′-deoxycytidine-5′-Triphosphate, 5-Formyl-2′-deoxyuridine-5′-Triphosphate, 5-Formylcytidine-5′-Triphosphate, 5-Formyluridine-5′-Triphosphate, 5-Hydroxy-2′-deoxycytidine-5′-Triphosphate, 5-Hydroxycytidine-5′-Triphosphate, 5-Hydroxymethyl-2′-deoxycytidine-5′-Triphosphate, 5-Hydroxymethyl-2′-deoxyuridine-5′-Triphosphate, 5-Hydroxymethylcytidine-5′-Triphosphate, 5-Hydroxymethyluridine-5′-Triphosphate, 5-Hydroxyuridine-5′-Triphosphate, 5-Iodo-2′-deoxycytidine-5′-Triphosphate, 5-Iodo-2′-deoxyuridine-5′-Triphosphate, 5-Iodocytidine-5′-Triphosphate, 5-Iodouridine-5′-Triphosphate, 5-Methoxycytidine-5′-Triphosphate, 5-Methoxyuridine-5′-Triphosphate, 5-Methyl-2′-deoxycytidine-5′-Triphosphate, 5-Methylcytidine-5′-Triphosphate, 5-Methyluridine-5′-Triphosphate, 5-Nitro-1-indolyl-2′-deoxyribose-5′-Triphosphate, 5-Propargylamino-2′-deoxycytidine-5′-Triphosphate, 5-Propargylamino-2′-deoxyuridine-5′-Triphosphate, 5-Propynyl-2′-deoxycytidine-5′-Triphosphate, 5-Propynyl-2′-deoxyuridine-5′-Triphosphate, 6-Aza-2′-deoxyuridine-5′-Triphosphate, 6-Azacytidine-5′-Triphosphate, 6-Azauridine-5′-Triphosphate, 6-Chloropurine-2′-deoxyriboside-5′-Triphosphate, 6-Chloropurineriboside-5′-Triphosphate, 6-Thio-2′-deoxyguanosine-5′-Triphosphate, 7-Deaza-2′-deoxyadenosine-5′-Triphosphate, 7-Deaza-2′-deoxyguanosine-5′-Triphosphate, 7-Deaza-7-Propargylamino-2′-deoxyadenosine-5′-Triphosphate, 7-Deaza-7-Propargylamino-2′-deoxyguanosine-5′-Triphosphate, 7-Deazaadenosine-5′-Triphosphate, 7-Deazaguanosine-5′-Triphosphate, 8-Azaadenosine-5′-Triphosphate, 8-Azidoadenosine-5′-Triphosphate, 8-Chloro-2′-deoxyadenosine-5′-Triphosphate, 8-Oxo-2′-deoxyadenosine-5′-Triphosphate, 8-Oxo-2′-deoxyguanosine-5′-Triphosphate, 8-Oxoadenoosine-5′-Triphosphate, 8-Oxoguanosine-5′-Triphosphate, Adenosine-5′-O-(1-Thiotriphosphate), Adenosine-S′-Triphosphate, ApA RNA Dinucleotide (5′-3′), ApC RNA Dinucleotide (5′-3′), ApG RNA Dinucleotide (5′-3′), ApU RNA Dinucleotide (5′-3′), Araadenosine-5′-Triphosphate, Aracytidine-5′-Triphosphate, Araguanosine-5′-Triphosphate, Arauridine-5′-Triphosphate, ARCA, Biotin-16-7-Deaza-7-Propargylamino-2′-deoxyguanosine-5′-Triphosphate, Biotin-16-Aminoallyl-2′-dCTP, Biotin-16-Aminoallyl-2′-dUTP, Biotin-16-Aminoallylcytidine-5′-Triphosphate, Biotin-16-Aminoallyluridine-5′-Triphosphate, CAP, Cidofovir-Diphosphate, CleanCap® Reagent AG, CleanCap® Reagent AG (3′ OMe), CleanCap® Reagent AU, CleanCap® Reagent AU, CleanCap® Reagent GG, CleanCap® Reagent GG, CleanCap® Reagent GG (3′ OMe), CleanCap® Reagent GG (3′ OMe), CpA RNA Dinucleotide (5′-3′), CpC RNA Dinucleotide (5′-3′), CpG RNA Dinucleotide (5′-3′), CpU RNA Dinucleotide (5′-3′), Cyanine 3-5-Propargylamino-2′-deoxycytidine-5′-Triphosphate, Cyanine 3-6-Propargylamino-2′-deoxyuridine-5′-Triphosphate, Cyanine 3-Aminoallylcytidine-5′-Triphosphate, Cyanine 3-Aminoallyluridine-5′-Triphosphate, Cyanine 5-6-Propargylamino-2′-deoxycytidine-5′-Triphosphate, Cyanine 5-6-Propargylamino-2′-deoxyuridine-5′-Triphosphate, Cyanine 5-Aminoallylcytidine-5′-Triphosphate, Cyanine 5-Aminoallyluridine-5′-Triphosphate, Cyanine 7-Aminoallyluridine-5′-Triphosphate, Cytidine-5′-O-(1-Thiotriphosphate), Cytidine-5′-Triphosphate, Dabcyl-5-3-Aminoallyl-2′-dUTP, dApdA DNA Dinucleotide (5′-3′), dApdC DNA Dinucleotide (5′-3′), dApdG DNA Dinucleotide (5′-3′), dApdT DNA Dinucleotide (5′-3′), dCpdA DNA Dinucleotide (5′-3′), dCpdC DNA Dinucleotide (5′-3′), dCpdC DNA Dinucleotide (5′-3′), dCpdG DNA Dinucleotide (5′-3′), dCpdG DNA Dinucleotide (5′-3′), dCpdT DNA Dinucleotide (5′-3′), dCpdT DNA Dinucleotide (5′-3′), Desthiobiotin-16-Aminoallyl-Uridine-5′-Triphosphate, Desthiobiotin-6-Aminoallyl-2′-deoxycytidine-5′-Triphosphate, dGpdA DNA Dinucleotide (5′-3′), dGpdA DNA Dinucleotide (5′-3′), dGpdC DNA Dinucleotide (5′-3′), dGpdC DNA Dinucleotide (5′-3′), dGpdG DNA Dinucleotide (5′-3′), dGpdG DNA Dinucleotide (5′-3′), dGpdT DNA Dinucleotide (5′-3′), dGpdT DNA Dinucleotide (5′-3′), dTpdA DNA Dinucleotide (5′-3′), dTpdA DNA Dinucleotide (5′-3′), dTpdC DNA Dinucleotide (5′-3′), dTpdC DNA Dinucleotide (5′-3′), dTpdG DNA Dinucleotide (5′-3′), dTpdG DNA Dinucleotide (5′-3′), dTpdT DNA Dinucleotide (5′-3′), dTpdT DNA Dinucleotide (5′-3′), Ganciclovir Triphosphate, GpA RNA Dinucleotide (5′-3′), GpC RNA Dinucleotide (5′-3′), GpG RNA Dinucleotide (5′-3′), GpU RNA Dinucleotide (5′-3′), Guanosine-3′,5′-bisdiphosphate, Guanosine-5′-O-(1-Thiotriphosphate), Guanosine-5′-Triphosphate, Inosine-5′-Triphosphate, Isoguanosine-5′-Triphosphate, mCAP, N1-Ethylpseudouridine-5′-Triphosphate, N1-Methoxymethylpseudouridine-5′-Triphosphate, N1-Methyl-2′-O-Methylpseudouridine-5′-Triphosphate, N1-Methyladenosine-5′-Triphosphate, N1-Methylpseudouridine-5′-Triphosphate, N1-Propylpseudouridine-5′-Triphosphate, N2-Methyl-2′-deoxyguanosine-5′-Triphosphate, N4-Biotin-OBEA-2′-deoxycytidine-5′-Triphosphate, N4-Methyl-2′-deoxycytidine-5′-Triphosphate, N4-Methylcytidine-5′-Triphosphate, N6-Methyl-2-Aminoadenosine-5′-Triphosphate, N6-Methyl-2′-deoxyadenosine-5′-Triphosphate, N6-Methyladenosine-5′-Triphosphate, Nucleoside-5′-Triphosphate Set, 06-Methyl-2′-deoxyguanosine-5′-Triphosphate, 06-Methylguanosine-5′-Triphosphate, pGp, Pseudoisocytidine-5′-Triphosphate, Pseudouridine-5′-Triphosphate, Puromycin-5′-Triphosphate, Thienocytidine-5′-Triphosphate, Thienoguanosine-5′-Triphosphate, Thienouridine-5′-Triphosphate, UpA RNA Dinucleotide (5′-3′), UpC RNA Dinucleotide (5′-3′), UpG RNA Dinucleotide (5′-3′), UpU RNA Dinucleotide (5′-3′), Uridine-5′-O-(1-Thiotriphosphate), Uridine-5′-Triphosphate, Xanthosine-5′-Triphosphate. Any combination of these nucleotides may be used as long as the generated primer extension products are inhibited from being used as a template.

In an embodiment of the invention the primer may comprise unusual nucleotides used in the reaction mixture. The unusual nucleotide may be at different positions in the primers. The unusual nucleotides in the primer prevent the primers to be copied as template, avoiding nonspecific priming and dimer formation.

In step (b) the polymerase used must be capable of incorporating the unusual nucleotide during modified complementary strand generation by primer extension, this generates a modified complementary strand which contains the unusual nucleotide, the polymerase must also be significantly inhibited from being able to use the modified complementary strand as a template and/or be significantly inhibited from being able to use the target specific primers as a template. The polymerase may be an archaeal DNA polymerase, or modified archaeal DNA polymerase or Family B polymerase such as Pfu DNA polymerase, Phusion DNA polymerase, Vent DNA polymerase, KOD DNA polymerase, Vent (exo-) DNA polymerase, Deep Vent (exo-) DNA polymerase, Deep Vent DNA polymerase, or Q5, or any combination thereof.

Step (b) or step (e) may be repeated one or more additional times, there may be a second set of the target-specific primers present in the reaction to either enrich by a one pass extension or multiple rounds resulting in amplifying the products. The second set of primers are capable of hybridising to the modified complementary strand generated from the first set of primers and or the original target polynucleotide. In another embodiment, to generate a complementary copy of the modified complementary strand, one may not need to add a second target specific primer, the hybridised first primer or partially extended first primers which are still hybridised to the modified complementary strand after step (b), upon adding second DNA polymerase, the hybridised first primer or partially extended first primers on the template of the modified complementary strand can be extended to make a full complementary copy of the modified complementary strand.

In one embodiment after step (b) the unusual nucleotide may be inactivated or otherwise removed such as by the addition of a phosphatase such as non-specific phosphatase including Shrimp Alkaline Phosphatase (rSAP), Antarctic Phosphatase or specific degradation enzymes such as Deoxyuridine triphosphate nucleotidohydrolase. With or without the inactivation or removal of the unusual nucleotide one or more additional polymerase may be directly added to the reaction mix, with or without additional dNTPs and other necessary reagents, which is known or believed to be able to use (be tolerant of) polynucleotides such as modified complementary strands which contain the unusual nucleotide which will allow for the modified complementary strand to be used as a template in further rounds of amplification. This additional polymerase may be a Family A polymerase such as Taq or a modified family b polymerase such as PhusionU or Q5U, or polymerases such as phi 29, bst, bsu, klenow or DNA polymerase I, or any combination thereof.

In another embodiment in step (b) a combination of polymerases may be used which have different properties such that one polymerase is able to incorporate an unusual nucleoside to generate a modified complementary strands but cannot use it as a template but a secondary polymerase is able to use the modified complementary strands as a template.

The target-specific primers in the first set and/or second set may comprise a unique molecular identifier (UMI) which is located between the 5′ tail portion and the 3′ target complementary portion, wherein UMI portion comprises at least three random or degenerated nucleotides, wherein during step (b) UMI assigns each modified complementary strands an unique sequence identifier such that during sequence analysis based on the unique UMI, sequenced PCR duplicates sharing the same UMI can be grouped into a family for the purpose of consensus read generation which allows for the comparison of sequences between family members which allow for the identification and correction of randomly produced process errors. The UMI may comprise a sequence that is between approximately 3 and 20 nucleotides in length.

Specifically, the UMI portion may comprise at least 3, 4, 5, 6, 7, 8, 9, 10, 11, 12, 13, 14, 15, 15-20, 20-30, or 31 or more completely or partially random or degenerated nucleotides or a predefined plurality of sequences, wherein during linear amplification step (b) UMI assigns each amplified strand with an unique sequence identifier such that during sequence analysis based on the unique UMI, the sequences sharing the same UMI are grouped into a family (FIG. 5).

The optional step (c) may comprise purifying the single-stranded linear amplification products. The function of the unusual nucleotide is to inhibit the amplification products from being used as a template requires that once this function is no longer required the unusual nucleotide may preferably be removed, made inert, or made otherwise non-functional which therefore allows for the modified complementary strands to be used as a template in subsequent downstream processes. The purification method removes the non-extended primers, this is important as any unused primer which persist into a second amplification reaction may still function as a primer which can have a negative effect on the quality of the final amplification products. Any method can be used; preferred method is purification by the use of magnetic beads, including but not limited to using Agencourt AMPure XP beads from Beckman coulter. After digestion or purification, the purified product may be immediately processed to step (e).

In the step (f), the PCR primers may comprise a second or third set of target-specific primers annealing to the linear amplification product, and universal primer which is related to the 5′ tail portion of primers of first set, or, if step (e) was completed two universal primers which can each anneal to a universal tail introduced in the first linear amplification(s) or a universal tail introduced in the second linear amplification. In step (c) the linear amplification product may be purified, for example beads purification, in the step (e) the PCR primers may include a second set of target specific primer annealing to the linear amplification product, and third set of target specific primers related to the 5′ part sequence of the first set.

As used herein “related” means comprising same sequence or similar sequence, for example similar may mean sharing at least 80-85%, 86-90%, 91%, 92%, 93%, 94%, 95%, 96%, 97%, 98% or 99% sequence identity. In one embodiment the universal primer is capable of hybridising to the 5′ tail portion of primers of first set. In one embodiment the universal primer is capable of hybridising to the 5′ tail portion of primers of second set. In one embodiment the universal primer is capable of hybridising to the copied part of the 5′ tail portion of the primers of the first set. In one embodiment the universal primer is capable of hybridising to the copied part of the 5′ tail portion of the primers of the second set.

The step (e) or (f) may comprise hybridising the modified complementary strands from either a single-stranded single-side amplification products or the barcoded opposing strand orientated linear amplification products to a second set of multiple target-specific primers which are capable of annealing to the linear amplification products generated from the first set of the target-specific primers.

UMI is preferably incorporated into primer extended target nucleic acids in the step (b), but UMI may be also incorporated into target nucleic acids in the step (e). In one embodiment, when the target-specific primer in the first set comprises only 3′ target complementary region without a 5′ tail, each primer in the second set comprises a 5′ tail portion, which comprises a UMI. In the steps (d-f) after removing the unreacted primers of the first set, the annealed primers of the second set may be extended on the templates generated from step (b), wherein the UMI is incorporated into the extended target nucleic acids. The extension may be done once or twice, or more than two times, which may be achieved by temperature cycling through denaturing, annealing and extension. In this embodiment, in the step (f) the PCR primers may include third set of target specific primer nested to the first set of target specific primer, and the universal primer related to the 5′ tail sequence of the primers of second set if the primers in the second set comprise a 5′ tail portion.

Alternatively, in the step (f) the PCR primers may include third set of target specific primer nested to the first set of target specific primer, and fourth set of target specific primers related to the 5′ part sequence of the second set if the primer in the second set comprises a bulge portion. Nested primers for use in the PCR amplification are oligonucleotides having sequence complementary to a region on a target sequence between reverse and forward primer targeting sites. One primer is called outer primer; its nested primer is called inner primer.

The nested inner primer may overlap by 1 or more nucleotides with its outer primer. In one embodiment, in the step (e) to enrich of the linear amplified product, the hybridised target-specific primers of the second set may be extended on the templates of the single-stranded single-side amplification products or the barcoded opposing strand orientated linear amplification products, the modified complementary strands. The extension reaction may be performed in the same reaction vessel as the linear amplification reaction vessel. After linear amplification with or without removing the unreacted primers of the first set and the unusual nucleotide, the target-specific primers of second set are added into the reaction, heat denatured, put to hybridisation/extension conditions. If the unusual nucleotide is not removed an additional polymerase must be added which is capable of using the modified complementary strand as a template. The extension conditions may include the same reagents in the linear amplification reaction. The extension may be performed at cycling conditions to extend the oligonucleotides several times, but preferably the extension is performed only once or twice. The extended double-strand products may be purified by any means known in the art, for example Qiagen PCR purification kit, or Agencourt Ampure XP kit.

In another embodiment, the target-specific primer in the second set may comprise a 5′ universal tail, wherein the 5′ universal tail portion of the target-specific primers may be hybridised with an affinity-labelled oligonucleotide complementary to the 5′ universal tail (FIG. 4). The affinity label may be biotin, the complex of the linear amplification products/target-specific oligonucleotides/biotin-labelled oligonucleotide may be captured by avidin solid supports.

The target specific primer of step (a) may be ordinary primer comprising target complementary sequence only or may be random. Preferably, the target specific primer of step (a) may comprise a 5′ tail portion and a 3′ target complementary portion. The 3′ target complementary portion is used to hybridise to the target sequence and prime DNA synthesise. The 5′ tail portion may comprise UMI, or/and sequence compatible to the followed amplification or/and sequencing process in a NGS platform (FIG. 1,2,3). For example, the 5′ tail portion may comprise sequence compatible to the primer used in the NGS. Alternatively, the target specific primer may comprise a 3′ target complementary portion, which is disrupted by a UMI, which is 3-20 nucleotides long. The 5′ tail portion is not complementary to the initial target sequence (FIG. 1). The 5′ tail portion of the primer may comprise UMI or/and sequence compatible to a NGS platform.

In step (a), either only one side of primers for a particular target is present in the reaction so that single-stranded linear amplification products are generated in step (b), or, both forward and reverse primers are present to generate barcoded opposing strand orientated linear amplification products from both the first and second strands. For single-stranded initial RNA target (referred to as first strand), the target specific forward primers complementary to the RNA template may be present in the reaction, the primers may also be random to allow for generation of randomly generated modified complementary strands, but no reverse primers are in the same reaction. For double stranded DNA templates, the target specific forward primers complementary to the first strands of the DNA templates are present in forward reaction, reverse primers may or may not be present in the same forward reaction. For single or double strand DNA templates the primer may also be partially or fully random to allow for random copying of the DNA sample to randomly generate modified complementary strands. This process may also be cycled so that 2 or more round of DNA amplification are allowed, this will result in a whole genome amplification where only the original DNA molecule is sampled each cycle as modified complementary strands will not be suitable templates. In some cases, this may result in partial copying of the modified complementary strands where the extension terminates at or in proximity to the unusual nucleotide.

When primers anneal to the target sequences, in the presence of reagents for linear amplification, step (b) is carried out. The linear single-side amplification or barcoded opposing strand orientated linear amplification can be isothermal amplification. Preferably, the linear single-side amplification or barcoded opposing strand orientated linear amplification is a thermal cycling amplification involving temperature cycling, including denaturing step, and annealing/extension step. The cycle number can be any suitable number, which may be between 1-100 cycles, for example 1 cycle, 2 cycles, 3 cycles, 4-10 cycles, 11-15 cycles, 16-20 cycles, 21-25, cycles, 26-30 cycles, 31-35 cycles, 36-40 cycles, 41-45 cycles, 46-50 cycles, 51-60 cycles or 61-100 cycles, or more.

After step (b), the reaction can immediately be processed to steps (d-f) without any purification and enrichment step. It is preferred that the remaining primers after the reaction of step (c) are kept at a considerably low level, therefore do not interfere the next step(s). One method to achieve this may be that the primers may be consumed in the linear amplification and reach to a very low level at the end of linear amplification. For this to happen, the primers added in the starting reaction must be in a very small amount, so that most primers are consumed after linear amplification. Alternatively, an optional purification or enrichment in step (d-e) may be carried out. Any purification method can be used to remove the unreacted primers, for example using beads to purify. Alternatively, enrichment of desired linear amplification product may be carried out.

Any enrichment method to enrich the linear amplification products can be used. The step (c) may comprise hybridising the linear amplification products to a second set of multiple target-specific primers. The second set of the target-specific primers may be the same as used in both step (a) or/and step (e-f). Alternatively, step (c) may use a different set of target specific primers or may not use target specific primers. In one embodiment, the hybridised second set of the target-specific primers may be extended on the templates of the linear amplification products (one pass extension). The extension reaction may be performed in the same reaction. The extended double-strand products may be purified by any means known in the art. The purified extended products are amplified in step (e-f). In the step (e-f) the primers used for amplification may comprise a first universal primer and a second universal primer, wherein the first universal primer comprises a sequence related to the 5′ tail portion sequence of primers in the first set, the second universal primer related to the 5′ tail portion sequence of the second set of the target-specific primers. Alternatively, in the step (e-f) the primers used for amplification may comprise a universal primer related to the first set of primer and a second set of multiple target specific primers, wherein the second set of multiple target specific primers capable of hybridising to the extended products of the first set of the primers, wherein the universal primer comprises a sequence related to the 5′ tail portion sequence of primers in the first set. Alternatively, in the step (e-f) the primers used for amplification may comprise a second set of multiple target specific primers, wherein the second set of multiple target specific primers capable of hybridising to the extended products of the first set of the primers, and third set of multiple target specific primers, which are nested primer relative to the first set, or are related to the 5′ part of bulge primer of the first set.

When the reaction mixture of the step (a) comprises target specific primers, the step (d-e) may comprise exonuclease treatment, for example exonuclease I, or/and purifying the product of step (c) to remove the unreacted primers, in the step (d) the purified product of step (b) is amplified by second set of target specific primers comprising 3′ priming sequences capable of hybridising to the purified linear amplified product of step (b) and third set of target specific primers comprising 3′ priming sequences which are identical or substantially identical to the first set of target specific primers (FIG. 1,2).

The linear amplification products may be enriched by hybridising probes on a solid support. The probes bind the desired linear amplification product specifically which are pre-bound to a solid support or are subsequently bound to a solid support. Since the first set of target-specific primers is used in linear amplification, the pairing second set of primers capable of hybridising to the single-stranded linear product of step (b) may be used in step (b) as probes to enrich the target sequence. The term “pairing” means, if one primer is forward primer, the pairing primer is reverse primer. The target specific primers may comprise a 5′ tail portion and a 3′ target complementary portion (FIG. 4. An affinity labelled oligonucleotide is complementary to the 5′ tail portion of the target specific primers. The affinity label may be biotin. The linear amplification products are hybridised to the target specific primers, which are then hybridised to the biotin labelled the oligonucleotide through the 5′ tail portion. Then the biotin labelled oligonucleotides are pulled out by streptavidin beads (FIG. 4). All unreacted primers, template DNA and non-specific products are removed by the enrichment. Particularly, if in the forward reaction the primers are forward primers, the linear amplification product from the forward reaction may be enriched by hybridising to the target specific reverse primers, which either comprise an affinity label, or comprise a 5′ tail portion which is hybridised to a universal oligonucleotide which comprises an affinity label.

The capture of the linear amplification products can be performed either on a solid phase or in liquid step. Typically, the capture operation of the enrichment will employ hybridisation to probes representing multiple target nucleic acids. On a solid phase, non-binding fragments are separated from binding fragments. Suitable solid supports known in the art include filters, glass slides, membranes, beads, columns, etc. If in a liquid phase, a capture reagent can be added which binds to the probes, for example through a biotin-avidin type interaction. After capture, desired fragments can be eluted for further processing.

In one embodiment after one or two or more cycles of amplification of a target polynucleotide in the presence of one or more unusual nucleotides multiple modified complementary strands may be generated where in the final round of amplification some or all modified complementary strands may have been partially copied where the extension terminates at or in proximity to the unusual nucleotide wherein the modified complementary strand and its partial copy are hybridised in a duplex.

In one embodiment prior in a step prior to the final round of amplification some or all of the unusual nucleotides are removed or otherwise made inert and replaced with standard nucleotides such that in the final extension a product can be generated with does or does not contain unusual nucleotides.

In one embodiment the gap(s) and/or nicks between the final amplification products where the unusual nucleotides have induced a stop or inhibition of extension may act as a point of selective digestion resulting in random, but specific, fragmentation of the modified complementary strand and its partial copies. The ends of the fragmentation may then be used as a point of ligation allowing for the incorporation of a second universal primer. The universal primer add by the random primer can then be paired with the second universal primer added by ligation and they can then be used for whole sample amplification.

In one embodiment the unusual nucleotide is dU wherein the agent of selective digestion is a combination of Uracil-DNA Glycosylase (UDG) or Uracil-N-Glycosylase (UNG), any fragment thereof or any functional alternative thereof, which generates an a-basic site and an endonuclease such as endonuclease IV or endonuclease VIII, or any fragment thereof or any functional alternative thereof, functionally capable of cleaving the a-basic site resulting in effective fragmentation of the whole genome amplified sample. Wherein the proportion of dU and a proportion of all nucleotides used allows you to modulate the average length of the DNA fragments generated by the fragmentation.

In another embodiment the unusual nucleotide is any combination of all 1, 2, 3 or all 4 of rATP, rCTP, rGTP and rUTP wherein each may all be used at the same or different ratios or combinations with or without other unusual nucleotides. Wherein the agent of selective digestion is any chosen from a list including but not limited to an RNAse, which may be, RNase A, RNase H, or RNase III or any fragment thereof or any functional alternative thereof, functionally capable of cleaving the at a rATP, rCTP, rGTP or rUTP site resulting in effective fragmentation of the whole genome amplified sample. Wherein the proportion of rATP, rCTP, rGTP and rUTP and a proportion of all nucleotides used allows you to modulate the average length of the DNA fragments generated by the fragmentation.

In some embodiments the proportion of unusual nucleotide used is based on the estimated average number of base pairs between incorporation events. In some cases, an idealist model may be used to estimate the number of base pairs between incorporation events wherein the target polynucleotide is a perfectly random distribution of A, T, C, and G nucleotides. In some cases, the unusual nucleotide is dUTP, and is used at some proportion as an alternative to dTTP. In one example, if dUTP and dTTP are used at a ratio of 1:99 in the presence of no unusual nucleotide alternative to dATP, dGTP, and dCTP then the final ratio of all 5 nucleotides will be 1:99:100:100:100 for a representative ratio for the unusual nucleotides relative to the other nucleotides of 1:399 with a total of 400. Therefore the chance of incorporating an unusual nucleotide on the perfectly random template is 1:400 when using a ratio of dUTP and dTTP of 1:99. The above approach of ratio choice can be used to influence the average maximum length of the partial copies of the modified complementary strands, this is due to the feature of extension inhibition of the unusual nucleotide resulting in the maximum length of the partial copies being equal to the average number of nucleotides between incorporation events. This can influence both the length and total copy number made depending on the use of polymerases at different stages of the protocol.

In some embodiments the first polymerase is a strand displacing polymerase which is able to incorporate the unusual nucleotide but is not efficiently able to use it as a template and would promote the strand displacement of partial copies of the modified complementary strands such that the length of the partial copies would maximise at the distance between unusual nucleotide incorporations as the maximum length possible would be for a random primer to anneal to a unusual nucleotide incorporation event and extent until it reach the next incorporation event. In some cases, the final extension may use a second polymerase which is a non-strand displacing polymerase which is able to use unusual nucleotide containing templates as a template whereby the polymerase can extend all partial copies beyond the unusual nucleotides until it reaches the end of the template or the 5′ end of the next partial copy. In which case the length of the final product are fully extended partial copies to the end of a modified complementary strand will be related to the ratio of the unusual nucleotide to all other nucleotides. In which case, the molarity of full partial copies is proportion to the number of modified complement strands.

In some embodiments the final extension may use a second polymerase which is a strand displacing polymerase which is able to use unusual nucleotide containing templates as a template whereby the polymerase can extend all partial copies beyond the unusual nucleotides until it reaches the end of the template and is able to displace all 3′ partial copies on the same modified complementary strand. In some cases, the unused primer will be remove prior to the use of the second polymerase. In which case both the average length and molarity of the final products which fully extended partial copies to the end of the modified complementary strand will be related to the ratio of the unusual nucleotide to all other nucleotides.

In another embodiment, these calculations become more complex when using non-perfect templates. In some cases, the non-perfect template may be polynucleotides representative of the human genome or a portion thereof in which case the ration of AT and CG nucleotides is approximately 60:40. Whereby, the average incorporation events are influenced by the ratio of the nucleotide the unusual nucleotide is equivalent to. In some cases, this may be further influenced by local regions of the genome which are very AT or GC rich.

In another embodiment, after amplification cycles the modified complementary strands and the partial copies are incubated with an agent to digest single-strand DNA. Wherein the agent of digestion is a mixture of one or more nucleases. Wherein the selected agent is chosen from a list of nucleases including but not limited to, exonuclease I, Thermolabile Exonuclease I, Exonuclease T, Exonuclease VII, RecJf, Mung Bean Nuclease, Nuclease P1, Nuclease Si, or any fragment thereof or any functional alternative thereof.

In one embodiment after the step (b) the modified complementary strand is hybridised to a second target specific primers with a 5′ affinity tag. The second primers are extended making an affinity tagged copy of the modified complementary strand the tagged double strand products are then affinity purified by capturing with solid phase support, such as beads. These purified products can then be used as templates for steps (e-f)

In another embodiment, the unusual nucleotide is incorporated into a process such as Illumina bridge amplification. In this process a target polynucleotide contains at least sequences which are identical to or designed to function equivalently to p5 and p7 sequences which allow polynucleotides to annealing to solid support, a flow cell. The standard Illumina bridge amplification process forms an exponential amplification of the target polynucleotide which anneals to the flow cell. When using unusual nucleotides, the first annealing and extension steps generates copies of the target polynucleotide which are covalently linked to the flow cell, this extension is done in the absence of the unusual nucleotide. Following this, 1 or more rounds of linear bridge amplification are done in the presence of an unusual nucleotide this results in the traditional exponential bridge amplification being converted into a linear amplification which will allow for the suppression of PCR artefacts. A change of polymerase and necessary reagents flowing over the flow cell can then allow for 1 or more rounds of exponential amplification, similar to normal bridge amplification, generating the final clusters for sequence by synthesis sequencing.

In step (f), primers used to generate double stranded PCR products may comprise target specific forward primers and target specific reverse primers. If the primers in the reaction of the step (a) are forward primers, another set of the target specific forward primers of step (e) may be nested primers in terms of forward primers of step (a). Alternatively, in step (f), primers used to generate double stranded PCR products may comprise a universal primer and a second set of multiple target specific primers. The second set of multiple target specific primers comprises either reverse primers or forward primers or both, wherein the universal primer comprises sequence related to the 5′ tail portion sequence or bulge portion of primers in the first set. If in the forward reaction of steps (a) the target specific primers are forward primers, which comprise 3′ target complementary portion and 5′ tail portion, the primers used in the forward reaction of step (e) comprise a second set of target specific reverse primers and universal primer, which are capable of targeting the 5′ tail portion of the primers used in steps (a). If in the reverse reaction of steps (a) the target specific primers are reverse primers, which comprise 3′ target complementary portion and 5′ tail portion, the primers used in the reverse reaction of step (d) comprise a second set of target specific forward primers and universal primer, which are capable of targeting to the 5′ tail portion of the primers used in steps (a). If the reaction of step (a) contains forwards and reverse primers each should have the same universal tails and in step (e) the primers comprise a second set of target specific forward and reverse primers and universal primer, which are capable of targeting the 5′ tail portion of the primers used in steps (a) (FIG. 1a).

The single-stranded starting molecule may be RNA, or single-stranded cDNA, or DNA. The double-stranded duplex may be genomic DNA, or any suitable dsDNA present in a sample or a product of previous amplification protocols. In step (a) the reaction mixtures may comprise one or two reactions: a forward reaction and/or a reverse reaction, or a mixed forward and reverse reaction. The forward reaction comprises a first set (forward set) of multiple target specific forward primers annealing to first strands of the multiple target sequences from one sample, and the reverse reaction comprises a first set (reverse set) of multiple target specific reverse primers annealing to the second strands of the multiple target sequences from the same one sample. The mixed forward and reverse reaction would contain a combination of primers annealing to the first and second strands. In the step (e or f), the primers used to generate amplification products may comprise a universal primer targeting 5′ tail portion of first set primers and another universal primer targeting 5′ tail portion of second set of primers if the step (e or f) comprises enriching the linear amplification products by hybridising and extension of the second set of the target-specific primers. Alternatively, the primers used to generate PCR products in the step (e or f) may comprise a universal primer targeting 5′ tail portion of first set primers and a second set of multiple target specific primers annealing to second strands of the multiple target sequences. Alternatively, the primers used to generate amplification products in the step (e or f) may comprise a universal primer targeting 5′ tail portion of first set primers and a third set of multiple target specific primers annealing to second strands of the multiple target sequences, wherein the third set of the target-specific primers (inner primers) is nested to the second set of the target-specific primers (outer primers). The universal primers in the forward and reverse reactions may be the same.

The reaction mixtures may comprise multiple reactions for more than one sample, which may be two samples, three samples or more than three samples, or more than 10 samples. Different samples may be process together in parallel. Each sample may comprise one or two reactions: forward reaction and/or reverse reaction, or a mixed forward and reverse reaction. All forward reactions or reverse reactions after linear amplification may be processed in one mixture in step (f or g) and followed steps.

In step (e or f), the PCR products may be purified and ready for sequencing, or may be further amplified in another PCR to add universal primers used for sequencing. In this step, all forward reaction and reverse reactions may be mixed and amplified by using universal primers, which target to the 5′ tail portion of the target specific primers used in step (a) or/and step (d).

Then the PCR products may be purified and size selected ready for NGS sequencing. The method further comprises analysing the NGS reads derived from the forward reaction and/or the reverse reaction or mixed forward and reverse reaction, which represent forward, reverse, or forward and reverse strands of target sequences, if necessary comprising generating error-corrected consensus sequences by (i) grouping into families containing the same UMI sequences; (ii) removing the target sequences of the same family having one or more nucleotide positions where the target sequence disagree with majority members, and (iii) examining if the same mutations appearing in the reactions, which represent different strands of a target sequence.

The method further comprises analysing the NGS reads derived from the forward reaction and the reverse reaction or the combined forward and reverse reaction, which represent two different strands of target sequences, comprising generating consensus sequences by grouping into families containing the same UMI sequences; and counting the numbers of families. This method provides a representative count for the numbers of original target nucleic acid molecules present in a sample.

The methods can be used to quantitate the starting molecules, although the single-side amplification or barcoded dual opposing strand orientated linear amplification may distort the number of the original target molecule number. Nevertheless, the counting of UMI families of a target sequence in comparison with other samples or comparing between forward reaction and reverse reaction, or between forward strands and reverse stands in a single reaction, may provide accurate counting information.

The present invention further provides a kit for performing a method according to one or more of proceeding methods, comprising: providing reaction mixture(s), each comprising an unusual nucleotide, a first set of multiple target specific primers annealing to multiple target sequences, wherein for any particular target sequence, forward primers are designed to hybridise to the first strands of the target sequences, reverse primers are designed to hybridise to the second strands of the target sequences, wherein the set of the target specific primers in reaction or reactions comprises forward primers, or, reverse primers, or, a mixture of forward and reverse primers; wherein the target specific primer(s) comprises a 5′ tail portion and a 3′ target complementary portion, both 5′ part and 3′ part of which are target specific sequences capable of hybridising to the target sequence; wherein the target-specific primer in the first set or second set comprises a UMI located between the 5′ tail portion and the 3′ target complementary portion, wherein the UMI portion comprises at least three random or degenerated nucleotides, wherein during step (a) UMIs assigns each extended strand an unique sequence identifier such that during sequence analysis based on the unique UMI, the sequences sharing the same UMI are grouped into a family; wherein the reaction mixtures are capable of carrying out linear amplification of the target sequences to generate single-stranded linear amplification products; optionally purifying or enriching reagents for purifying or enriching the single-stranded linear amplification products; and PCR amplifying reagents for amplifying the single-stranded linear amplification products using primers to generate double-stranded PCR products; wherein the primers and reagents are described in the proceeding methods.

A target-specific primer may comprise a UMI between 5′ universal tail and 3′ target complementary portion. The purpose of the UMI is twofold. First the assignment of a UMI to each DNA template molecule. The second is the amplification of each uniquely tagged template, so that many daughter molecules with the identical UMI sequence are generated (defined as a UMI family). If a mutation pre-existed in the template molecule used for amplification, that mutation should be present in every daughter molecule, or a majority of daughter molecules, containing that UMI.

A target-specific oligonucleotide may further comprise a fixed multiplexing barcode sequence between 5′ universal tail and 3′ target complementary portion or in the bulge portion. The barcode sequence and UMI may both be present; barcode can be located at 5′ or 3′ of UMI.

The universal primers may contain one, or two, or more terminal phosphorothioates to make them resistant to any exonuclease activity. They may also contain 5′-grafting sequences necessary for hybridization to NGS flow cell, for example the Illumina GA Ix flow cell. Finally, they may contain an index sequence between the grafting sequence and the universal tag sequence, or, between the universal tag sequence and a target specific sequence. This index sequence enables the PCR products from multiple different individuals to be simultaneously analysed in the same flow cell compartment of the sequencer.

The target nucleic acid sequence may comprise a nucleic acid fragment or gene which contains variant nucleotide(s), and may be selected from the group consisting of disorder associated SNP/deletion/insertion, chromosome rearrangement, trisomy, or cancer genes, drug resistance gene, and virulence gene. The disorder-associated gene may include, but is not limited to cancer-associated genes and genes associated with a hereditary disease. Possible variants may be known to be or be correlated to a disease state or be newly identified variants.

The variant nucleotide(s) in the diagnostic region of the target polynucleotide sequence may include one or more nucleotide substitutions, chromosome rearrangement, deletions, insertions and/or abnormal methylation.

DNA methylation is an important epigenetic modification of the genome. Abnormal DNA methylation may result in silencing of tumour suppressor genes and is common in a variety of human cancer cells. In order to detect the presence of any abnormal methylation in the target polynucleotide, a preliminary treatment should be conducted prior to the practice of the present method. Preferably, the nucleic acid sample should be chemically modified by a bisulphite treatment, which will convert cytosine to uracil but not epigenetically modified cytosine (i.e., 5′-methylcytosine, which is resistant to this treatment and remains as cytosine), an enzymatic treatment such as the combination of a TET family member with APOBEC which results in the conversion of unmethylated C to U but not the methylated cytosine, or chemical conversion by ‘TAPS chemistry’. With these modifications, the method of this invention can be applied to the detection of abnormal methylation(s) in the target nucleic acid.

The present invention provides a method of analysing a biological sample for gene expression. In one embodiment, the UMI is assigned to every linear amplification strand and subsequently is identified during sequence analysis. In another embodiment a UMI is assigned in a linear amplification which use a first linear amplification product as a template.

The present invention provides a method of analysing a biological sample for the presence and/or the quantity of mutations or polymorphisms at a single or at multiple loci of different target nucleic acid sequences. In another aspect, the present invention provides a method of analysing a biological sample for chromosomes abnormality of, for example, trisomy. The amplification and enriching step or steps may be followed by next generation sequencing, qPCR, digital PCR, microarray, or other low or high throughput analysis. The number of multiplexing of target loci may be more than 1, or more than 5, or more than 10, or more than 30, or more than 50, or more than 100, more than 1000, or more.

One limitation of traditional PCR methods is that when a mutant is very rare in a sample, for example one or two mutants are present in the sample, in order to get strand aware information the sample must be divided into two separate reactions, after dividing the sample nucleic acid into two reactions, only one reaction may contain the mutant. This means that comparison of the mutation in two strands sequences in the two reactions is impossible. However, the specificity can be increased by requiring more than one mutation sequencing reads in one reaction for mutation identification—the probability of introducing the same artefactual mutation twice or three times would be extremely low.

Instead of matching sequencing reads of forward and reverse reactions, more than one mutation sequencing reads in different UMI molecules in forward or reverse reaction may also be classified as mutant positive, as during single-side linear amplification step, the same artefacts appear more than twice would be very rare.

The use of barcoded opposing strand orientated linear amplification allows an improvement on traditional PCR whereby you are able to selectively amplify the first and second strands of a target polynucleotide in a single reaction and maintain strand aware information in the data generated by massively parallel sequencing. The forward strand targeting primers linearly amplify the forward strand and the reverse strands targeting primers linearly amplify the reverse strand. By the use of the unusual nucleotide the generated linear amplification products cannot be used as a template by the opposing primers. After any necessary or useful purification steps a second set of amplification can further enrich the dual opposing strand orientated linear amplification products. A universal primer designed to amply from the universal tail on the first amplification primers, a forward strand primer designed to anneal to and amplify the reverse strand linear product in combination with the universal primer, and a reverse primer designed to anneal to and amplify the forward strand linear amplification products in combination with the universal primer. The second forward and reverse primers may have the same universal primer which will in inhibit unwanted PCR products by any products forming internal hairpins preventing their use as template molecules.

The release of cell-free DNA into the bloodstream from dying tumour cells has been well documented in patients with various types of cancer. Research has shown that circulating tumour DNA can be used as a non-invasive biomarker to detect the presence of malignancy, follow treatment response, or monitor for recurrence. However, current methods of detection have significant limitations. Next Generation Sequencing (NGS) methods have revolutionised genomic exploration by allowing simultaneous sequencing of hundreds of billions of base pairs at a small fraction of the time and cost of traditional methods. However, the error rate of ˜ 1% results in hundreds of millions of sequencing mistakes, which is unacceptable when aiming to identify rare mutants in genetically heterogeneous mixtures, such as tumours and plasma. The methods of this invention can be implemented to help overcome these limitations in sequencing accuracy. Mutation harbouring cfDNA can be obscured by a relative excess of background wild-type DNA; detection has proven to be challenging. The method greatly reduces errors by independently tagging and sequencing each original DNA duplex through dual opposing strand orientated linear amplification.

The methods of the present invention can substantially improve the accuracy of massively parallel sequencing. It can be implemented through either UMI in target specific primer and can be applied to virtually any sample preparation workflow or sequencing platform and can be applied to any situation where PCR between opposing primers is unwanted or where amplification of a generated template is unwanted. The approach can easily be used to identify rare mutants in a population of DNA templates. One of the advantages of the strategy is that it yields the number of templates analysed as well as the fraction of templates containing variant bases. The two strands of one target template in sample in one tube, each is uniquely tagged and independently sequenced. Comparing the sequences of the two strands results in either agreement to each other or disagreement. The agreement gives the confidence to score a mutation as true positive. Artefactual mutations introduced during PCR amplification are detectable as errors, if both strand sequences of two populations does not agree to each other.

In one embodiment, during the linear amplification and UMI tagging, many “families” of molecules are created, each of which arose from a single strand of an individual DNA molecule. After sequencing, members of each PCR family are identified and grouped by virtue of sharing the identical UMI tag sequence. The sequences of uniquely UMI tagged family and two strands of target sequences are then compared to create a consensus sequence. This step filters out random errors introduced during sequencing or PCR to yield a set of sequences, each of which derives from an individual molecule of single-stranded DNA.

Next, sequences belonging to the two complementary strands of each target are identified by searching for complementary sequences among sequencing reads. Following partnering of the two strands, the sequences of the strands are compared. A sequence base at a given position is kept only if the read data from each of the two strands is significantly similar or matches perfectly. The ratio of any mutation among the two strands are also compared; only the similar ratio of the numbers of mutant and normal sequence among the two strands indicates true mutation positive. Comparing the sequences obtained from both strands eliminates errors introduced during the first round of PCR where an artefactual mutation may be propagated to all PCR duplicates of one strand and would not be removed by single strand sequencing filtering alone.

In addition to their application for high sensitivity detection of rare DNA variants, the UMI in the target specific primer can also be used for single molecule counting to accurately determine absolute or relative DNA or RNA copy numbers. Because tagging occurs before major amplification, the relative abundance of variants in a population can be accurately assessed given that proportional representation is not subject to skewing by amplification biases.

Reagents employed in the methods of the invention can be packaged into kits. Kits include the primers, in separate containers or in a single master mixture container. The kit may also contain other suitably packaged reagents and materials needed for extension, amplification, enrichment, for example, buffers, dNTPs, the unusual nucleotide, and/or polymerizing means; and for detection analysis, for example, and enzymes, as well as instructions for conducting the assay.

The methods of the present invention greatly reduce errors by: tagging two strands of any target sequences (or one target sequence and one artificial unique template with UMI) derived from one or two separate initial preparations with identifiable sequence signatures; tagging each target sequence with UMI; barcoded opposing strand orientated linear amplification sequencing the two strands. In addition, the methods provide uniform amplification of multiple target sequences. Analysis provides error-corrected consensus sequences by grouping the sequenced uniquely tagged sequences or linked two amplicons into families containing the same pair of the two amplicons, which is further grouped into families containing the same UMI sequences; removing the target sequences of the same family having one or more nucleotide positions where the target sequence disagree with majority members in a family; and same mutations appearing in the two populations would be the highest confidence true mutations.

The method can be used for detecting mutation in any sample such as FFPE or blood. The accurate counting of sequencing reads which reflect the original molecules present in a sample provides information for copy number variations or for prenatal test for chromosome abnormality.

### BRIEF DESCRIPTION OF THE DRAWINGS

FIG. 1a depicts a schematic of an illustrative embodiment of the present invention. In a combined forward and reverse reaction, a set of multiple forward and multiple reverse primers are hybridised to the first strands and second strands of the target polynucleotide. In the presence of an unusual nucleotide, in this embodiment dUTP, a polymerase capable of incorporating the unusual nucleotide during primer extension generating modified complementary strand and is unable to use the modified complementary strands as a template, and other necessary reagents for linear amplification, barcoded opposing strand orientated modified complementary strands are generated. The linear amplification may be thermal cycling amplification with one sided or two sided primers. In the linear amplification both strands of a target sequence may be amplified if primers targeting both strands are used. For this example if there are 7 cycles of linear amplification then the original strands are amplified up to 7 times, but no PCR is expected to have occurred. Each primer has a random sequence identifier (UMI) such that each amplified modified complementary strand has a unique molecular identifier, which can be identified during sequence analysis. The barcoded single strand linear or barcoded opposing strand oriented linear strands may be enzymatically treated to remove unreacted primer or unused unusual nucleotides, or purified or enriched. This step is optional as it may be not necessary if the primers are greatest diminished after linear amplification or if an additional polymerase is added which is capable of using modified complementary strands as a template. The modified complementary strands are then used as a template in a PCR reaction using forward primers (may be universal primers or target specific primers) and target specific reverse primers. The PCR products may be further amplified in another PCR to add universal primers used for next generation sequencing. The final PCR products may be purified and size selected.

FIG. 1b. In a linear amplification, in heavily tiled regions head-to-head linear primers and the use of an unusual nucleotide have a synergistic effect in reducing nonspecific PCR products while also allowing for fully tiled linear amplification of the target genomic regions. In the following PCR, by using head-to-head PCR primers in combination of universal primer with tail sequence of linear primer, we are able to generate overlapping tiled amplicons allowing for easy whole gene coverage where each molecule contains a UMI to help improve the accuracy of mutation detection.

FIG. 2. depicts a schematic of an illustrative embodiment of the present invention. In a combined forward and reverse reaction, a set of multiple forward and multiple reverse primers are hybridised to the first strands and second strands of the target polynucleotide. In the presence of an unusual nucleotide, in this embodiment dUTP, a polymerase capable of incorporating the unusual nucleotide during primer extension generating modified complementary strand and is unable to use the modified complementary strands as a template, and other necessary reagents for linear amplification, barcoded opposing strand orientated modified complementary strands are generated. The linear amplification may be thermal cycling amplification with one sided or two-sided primers. In the linear amplification both strands of a target sequence may be amplified if primers targeting both strands are used. For this example if there are 7 cycles of linear amplification then the original strands are amplified up to 7 times, but no PCR is expected to have occurred. Each primer has a random sequence identifier (UMI) such that each amplified modified complementary strand has a unique molecular identifier, which can be identified during sequence analysis. The barcoded single strand linear or barcoded opposing strand oriented linear strands may be enzymatically treated to remove unreacted primer or unused unusual nucleotides, or purified or enriched. This step is optional as it may be not necessary if the primers are greatest diminished after linear amplification or if an additional polymerase is added which is capable of using modified complementary strands as a template. The modified complementary strands are then used as a template in a second linear amplification reaction using target specific reverse primers, this may or may not in the presence of a second unusual nucleotide, a polymerase capable of incorporating the second unusual nucleotide during primer extension generating modified copies of the modified complementary strand and is unable to use the modified copies of the modified complementary strands as a template, and other necessary reagents for linear amplification. The modified copies of the modified complementary strands are then used as a template in a PCR reaction using a third set of primers (may be universal primers or target specific primers). The PCR products may be further amplified in another PCR to add universal primers used for next generation sequencing. The final PCR products may be purified and size selected.

FIGS. 3a and b depict schematics of an illustrative embodiment of the present invention and its application using DNA which has undergone deamination of cytosine to uracil, or, a equivalently different nucleotide as input nucleic acids. This example depicts the use of bisulfite conversion. After chemical and/or enzymatic conversion the modified input nucleic acids are used as a template for generation of linear amplification products, using any disclosed method, such as the method in FIG. 1 or FIG. 2. The first amplification step may not use an unusual nucleotide and will not generated modified complementary strands. The second linear amplification may use modified nucleotides and during this step the modified complementary strands may be generated. The first and the second linear amplification steps may generate modified complementary strands and modified copies of modified complementary strands when unusual nucleotides are used in both steps. The “x” represents an unusual nucleotide.

FIG. 4 depicts primers and affinity labelled oligonucleotides. (A) a primer with a 5′ tail portion and 3′ target complementary portion. (B) primer comprises a 5′ tail portion, a UMI 3′ to the tail portion and a 3′ target specific portion. (C) primer comprises a 5′ affinity tag, a tail portion 3′ to the tag, a UMI 3′ to the tail portion and a 3′ target specific portion bound to a solid surface in this example a bead is depicted which itself is bound to an affinity tag binding moiety. (D) affinity labelled oligonucleotide hybridises to the 5′ tail portion of a primer, the affinity label is attached to a solid surface in this example a bead is depicted.

FIG. 5 depicts a schematic of an illustrative embodiment of the present invention and how it allows for the preservation of strand aware information. (A) Primers contain a UMI which gives with modified complementary strand a UMI and when used in barcoded opposing strand orientated linear in the absence of an unusual nucleotide will undergo PCR based amplification, resulting in copies of the first and second strand have the same UMI and same universal tails. After an optional purification a second round of PCR amplification with primers which are a mixture of target specific primers, and, universal primer which bind to the universal tail of the first target specific primers, are used to generate a second round of PCR products. These PCR products will lose all strand aware information. As the first primers were able to undergo PCR they would have made copies equivalent to both the original first and second strands, so any further PCR will not be able to differentiate which the original strand was. (B) when the same reaction occurs in the presence of the unusual nucleotide the first barcoded opposing strand oriented linear reaction is only a linear amplification. When these modified complementary strands are used as a template with the second primers for PCR the original strand information is maintained. This allows for strand aware PCR amplification without a need to divide a sample.

FIG. 6 In a single reaction both strands of a double strand target DNA molecule are amplified. In (A) without using unusual nucleotides, whereas in (B) with using unusual nucleotides. This amplification is barcode opposing strand oriented linear amplification generating modified complementary strands. Primers contain a UMI which gives with modified complementary strand a UMI. The primers in the linear amplification comprise the first 5′ universal tail sequence. The linear amplification (B) is further enriched by hybridising a second set of target specific primers and undergoing either PCR amplification, one-pass extension and purifying or capturing on beads. The primers in the PCR amplification comprise the second 5′ universal tail sequence, wherein the first and second universal tail sequence are different. The enriched PCR products are further amplified using primers containing sequences compatible to an NGS platform. The PCR are then sequenced on any suitable next generation sequencer. The generated sequencing data is then analysed and the reads which originated from the first and reads originating from the second strand are identified, these reads are then used to generate error-corrected consensus sequences by (i) grouping into families containing the same set of random UMIs; (ii) using these groups to removing the nucleotide sequences which differ to the expected normal sequence and are in a minority of the sequence reads which belong to a single family this generates a consensus read (iii) the consensus reads are then compared together and against a reference sequence where true mutations are those present in either multiple consensus reads from one strand or from consensus reads from both first and second strands. In (B) Strand information is NOT lost in products. When looking for mutations, any mutations found can be attributed to sense or antisense strands. In (A) Strand information lost in products as both first and second strands can act as a template for first strand specific primers, or second strand specific primers. When looking for mutations, any mutations found cannot be attributed to sense or antisense strands

FIG. 7 depicts a schematic of an illustrative embodiment of the present invention. A) Depicts two non-specific primers binding to a region of the starting nucleic acid. During an amplification reaction these two primers would be expected to produce exponential amplification of the region between the two primers. This amplification is unwanted. B) Show that the same two primers in the presence of the unusual nucleotide will be significantly inhibited from exponentially amplifying the region between the two primers

FIG. 8 depicts a schematic of an illustrative embodiment of the present invention. A) Depicts a traditional method for whole sample copying/amplification by a process of strand displacement amplification. Where copies of nucleic acids are themselves copied one, or more than one times. B) Depicts the same reaction in the presence of an unusual nucleotides. Whereby the modified complementary strands are not able to be efficiently copied. This will help to reduce the bias of the amplification of the starting nucleic acids. This may use DNA or RNA starting material. The “x” represents an unusual nucleotide.

FIG. 9 depicts results demonstrating an embodiment of the present invention. Following the method in example 1, the generated qPCR data is shown here. Relative to an unamplified gDNA control vent exo- was able to generate PCR products resulting in a drop in measure Ct value, these PCR products were not significantly effected by UDG+Endo VIII digestion. A PCR reaction including an unusual nucleotide resulted in a significantly smaller change in Ct value relative to the control, after UDG+Endo VIII digestion the Ct value returned to normal levels indicating that linear amplification products were made and they incorporated then unusual dUTP nucleotide and these products were destroyed by incubation in the presence of UDG+Endo VIII. A linear amplification reaction in the presence of dTTP produced products with a similar Ct value drop equivalent to a PCR reaction in the presence of the unusual nucleotide which demonstrates that the PCR was acting as a linear amplification, these products were not sensitive to UDG+Endo VIII digestion. Finally, a linear amplification in the presence of an unusual nucleotide produced a drop in Ct value similar to PCR in the presence of an unusual nucleotide, and these products were also sensitive to UDG+Endo VIII digestion.

FIG. 10 depicts results demonstrating an embodiment of the present invention. Following the method in example 2, the generated qPCR data is shown here. (A) visualisation of the qPCR data demonstrating an increase in Ct concordant with an increase in dUTP percentage in the PCR reactions. The inhibition of the PCR plateaus between 60-80% dUTP in the presence of 40-20% dTTP. The PCR Ct approach the linear amplification Ct values demonstrating that this reaction has transformed from a exponential PCR to a linear reaction. (B) visualisation of the level of inhibition of PCR. The copy number of the PCR product at 20% dUTP decreased by 500 fold, at 40% decreased by 3000 fold, at 60% by 6500 fold. This indicates that significant levels of inhibition can be achieved with 40-60% dUTP.

FIG. 11 depicts results demonstrating an embodiment of the present invention. Following the method in example 3, the sequencing data analysis is shown. The number of sequencing reads for the sample generated using dUTP in the barcoded opposing strand oriented linear reaction versus the equivalent final PCR products generated using no dUTP. The majority of the target regions do not use opposing primers and as such do not demonstrate a significant difference between the presence and absence of dUTP (blue spots). A selection of target regions using opposing primers, these sites have a noticeably lower sequencing depth in the presence versus absence of dUTP (orange spots). This indicates that the behaviour of dU in being able to inhibit PCR results in a significant effect in the suppression of unwanted PCR during the generation of a next generation sequencing library.

FIG. 12 depicts results demonstrating an embodiment of the present invention. Following the method in example 4, the sequencing data analysis is shown. This data shows the detected and the expected allele frequency for the mutations covered by the target specific primers used in this example on test material.

FIG. 13 depicts an embodiment for targeted amplification or random amplification. The target regions are linearly amplified in presence of unusual nucleotides using first primer which is target specific primers with the same 5′ tail, or with two different tails, wherein one tail is attached to one of the paired primers, another tail is attached to another primer of the paired primers in the opposite direction. When random regions are linearly amplified, the first primer is a random primer with 3′ random sequence, with or without 5′ universal tail sequence. After linear amplification, a second set of primers comprising target specific primers which are capable of hybridising to the modified complementary strands, wherein the target specific primers have a different 5′ tail sequence relative to the first primer, and universal primers having the same sequence as 5′ tail of the first primers is added. Using the second set of primers, the second DNA polymerase amplifies the modified complementary strands.

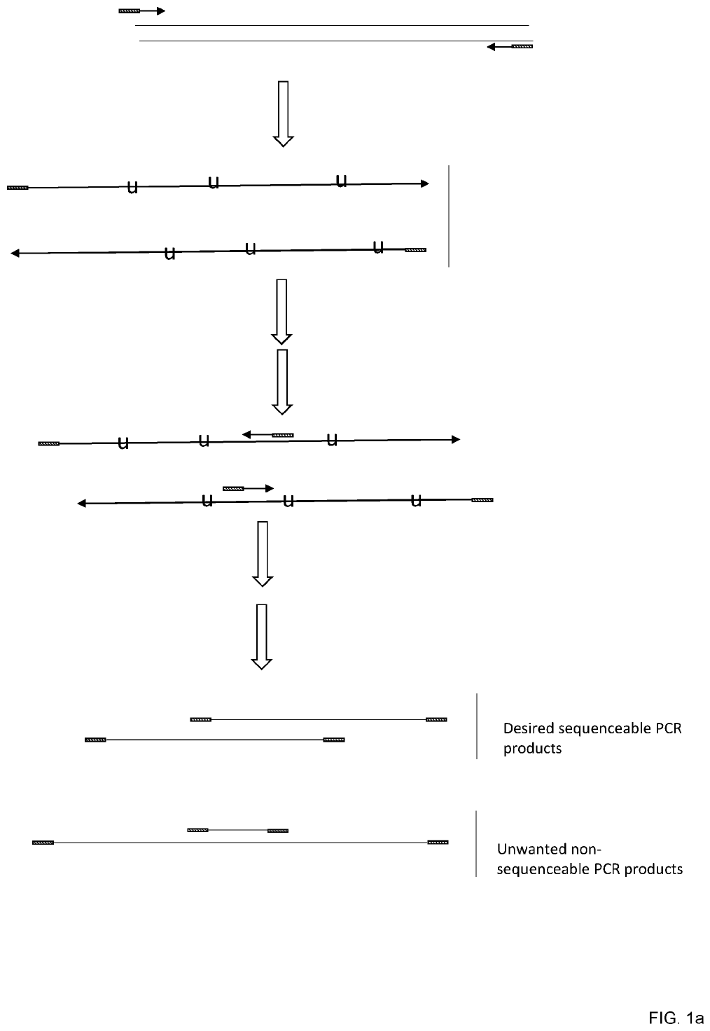

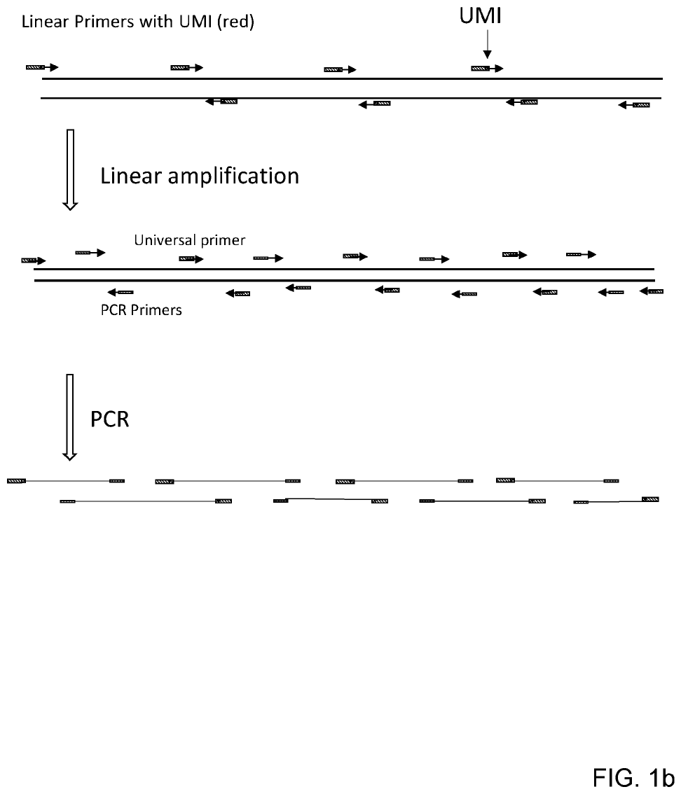

Alternatively, after linear amplification, a second DNA polymerase is directly added to the same linear reaction and performs one pass extension (one cycle or more cycles) to allow making a full copy of the modified complementary strand. After making the double stranded modified complementary strands, which may be optionally purified, the strands are amplified using universal primers (second primer) having the same sequence as tail of the first primers. Alternatively, after linear amplification, the linear amplification product is optionally purified to remove unused primers. Without adding target specific second primers or second random primers, the second DNA polymerase extends the hybridised first primers or partially extended first primers inherited from linear amplification step on the template of the modified complementary strands to make a full complementary copy of the modified complementary strands. In the same reaction vessel, the universal second primer is used to amplify the modified complementary strands. The universal second primer has the sequence substantially identical to the 5′ tail sequence of the first primers.

FIGS. 14 A) and B) depicts a schematic of illustrative embodiments of the present invention for targeted amplification of genetic information from unconverted gDNA and targeted amplification of epigenetic information from converted DNA. The target regions are linearly amplified in the presence of unusual nucleotides, in this depiction including but not limited to 5-Methyl-2′-deoxycytidine-5′-Triphosphate, using first primers which are target specific primers with universal tails. After linear amplification the modified complementary strands and original target nucleic acids are deaminated by either or combined chemical and/or enzymatic processes. Optionally, in some cases, the deaminated original strands and or modified complementary strands may be linearly amplified with or without unusual nucleotides using a second set of primers comprised of a 3′ targeting or random regions, with or without UMIs, and a 5′ universal priming site. Using a second, or third, set of primers and a second, or third, polymerase the modified complementary strand and deaminated original strand target polynucleotide or copies of deaminated original strands, or second linear amplified polynucleotides are further amplified. Alternatively, only the modified complementary strand or original deaminated target polynucleotide are amplified, or, the sample is divided into two different reactions before or any amplification step and the modified complementary strand and original deaminated target polynucleotide are individually amplified.

FIG. 15 depicts results demonstrating an embodiment of the present invention. Following the method in example 8, the analysis of the sequencing data is shown. This data shows the detected and the expected allele frequency for the mutations covered by the target specific primers used in this example on FFPE lung cancer samples. It also displays data for the detected mutations using two alternative technologies which demonstrate high levels of accuracy of the present invention relative to these other data.

FIG. 16 depicts a schematic of an illustrative embodiment of the present invention for targeted amplification or random amplification. The target polynucleotide is linearly amplified in the presence of unusual nucleotides using first primer which is random primer with 3′ random sequence, with or without 5′ universal tail sequence. In some cases, the first primer is targeted specific primers. In some cases, the first linear amplification is 2 or more cycles of amplification. In second and subsequent cycles of amplification the modified complementary strands will in turn be partially copied by a primer annealing and being extended until it reaches an unusual nucleotide which it cannot copy which results in partially copied modified complementary strands. In some cases, if the unusual nucleotide is removed or otherwise made inert and replaced with a standard nucleotide the final cycle extension products will not have unusual nucleotides in their formation. The unusual nucleotide may then be used for selective digestion resulting in the fragmenting of the modified complementary strands at the site of unusual nucleotide incorporation which is the same point at which copying was terminated. In some cases, these fragmented modified complementary strands and partial copy duplexes may subsequently be used for a substrate in a ligation reaction during which a universal primer can be ligated to all double-strand DNA ends generated by the fragmentation event. The polynucleotide with two universal primer sites can then be used in amplification reactions allowing the generation of polynucleotides suitable for NGS or massively parallel sequencing.

FIG. 17. depicts a schematic of an illustrative embodiment of the present invention in how the use of unusual nucleotides can result in bias of final molecules to a range of lengths. The target polynucleotide is linearly amplified in the presence of unusual nucleotides, wherein the unusual nucleotide is at 3 different percentages in this example M, M*2 and M*4, using first primer which is random primer with 3′ random sequence, with or without 5′ universal tail sequence. In some cases, the first primer is targeted specific primers. In some cases, the first linear amplification is 2 or more cycles of amplification. In second and subsequent cycles of amplification the modified complementary strands will in turn be partially copied by a primer annealing and being extended until it reaches an unusual nucleotide which it cannot copy which results in partially copied modified complementary strands. In some cases, the polymerase will have strand displacement ability such that the partial copies of the modified complementary strands lengths will be maximised towards the expected average number of bases between incorporation events. In some cases, a second extension reaction will contain a second polymerase which is capable of using unusual nucleotide containing templates as a template which does not have strand displacement activity and will allow for the full copying of molecules whose length is related to the proportion of unusual nucleotide. Wherein the length is, on average, 400/M bp, 400/(M/2) bp, or 400/(M/4) bp with only the very 3′ partial copy fully copied. In some embodiments, a second extension reaction will contain a second polymerase which is capable of using unusual nucleotide containing templates as a template and also has strand displacement activity and will allow for the full copying of molecules whose length and copy number is related to the proportion of unusual nucleotide. Wherein L is the average length of all modified complementary strands and the final fully copy lengths are, on average 400/M bp with L/(400/M) copies, 400/(M*2) bp with L/(400/(M*2)) copies, or 400/(M/4) bp with L/(400/(M/4)) copies.

### EXAMPLES

TABLE 1
Details of all Oligos

| | Seq | |
|---|---|---|
| ID | ID | Sequence |
| 1-001 | 1 | ACGCAGGTCGTATTGGGCGCCTG |
| 1-002 | 2 | GGGTCATTGATGGCAACAATATCC |
| 1-003 | 3 | [CY5]ACCAGAGTTAAAAGCAGCCCTGGTG[BHQ2] |
| 1-004 | 4 | ACACTCTTTCCCTACACGACGCTCTTCCGATC*T |
| 1-005 | 5 | Pool of 110 linear amplification |
| | | primers |
| 1-006 | 6 | Pool of 110 PCR amplification primers |
| 1-007 | 7 | AATGATACGGCGACCACCGAGATCTACACCGGAACAA |
| | | ACACTCTTTCCCTACACGACGCTCTTCCGATC*T |
| 1-008 | 8 | CAAGCAGAAGACGGCATACGAGATCATTCCAAGTGAC |
| | | TGGAGTTCAGACGTGTGCTCTTCCGAT*C*T |
| 1-009 | 9 | Pool of 110 linear amplification |
| | | primers |
| 1-010 | 10 | Pool of 160 linear amplification |
| | | primers |
| 1-011 | 11 | PCR amplification primers |
| 1-012 | 12 | GTGACTGGAGTTCAGACGTGTGCTCUUCCGAUCUNNN |
| | | NNNNNNNNNNN*N |
| 1-013 | 13 | ACACTCTTTCCCTACACGACGCTCUUCCGAUCUNNNN |
| | | NNNNNNNNNN*N |
| 1-014 | 14 | AGACGTGTGCTCTTCCGATCTNNNNNNNNNNNNNN*N |
| 1-015 | 15 | CTCTTTCCCTACACGACGCTCTTCCGATCT |
| 1-016 | 16 | AGATCGGAAGAGCGTCGTGTAGGGAAAGAGTGTTTGT |
| | | TCCGGTGTAGATCTCGGTGGTCGCCGTATCATT |

### Example 1

Using deoxyribonucleic acid (DNA) as the target polynucleotide for determining the ability for a DNA polymerase to incorporate dU into a primer extension product but not be able to use the modified polynucleotide as a template. PCR mixes were prepared using either a single primer, or a pair of opposing primers such that either a linear amplification or exponential amplification would occur in the presence of traditional nucleotides, but only linear amplification would occur in the presence of an unusual nucleotide, in this example the unusual nucleotide is dUTP. These reactions were set up with a combination of dATP, dTTP, dCTP and dGTP, or, dATP, dUTP, dCTP and dGTP. Half of each sample was digested by UDG+Endo VIII which can only fragment DNA containing dU. These reactions were then bead purified and the copy number of the resultant amplified polynucleotides determined by qPCR and compared between the digested and undigested aliquots. This demonstrated that DNA polymerases are able to incorporate dU during primer extension but cannot use the subsequent modified complementary strands as a template.

### Materials

Target polynucleotide, human gDNA (ENZ-GEN117-0100)
Vent exo-DNA polymerase (NEB, M0257S)
Vent exo-DNA polymerase buffer (NEB, B9004S)
dATP Solution (NEB, N0440S)
dCTP Solution (NEB, N0441S)
dGTP Solution (NEB, N0442S)
dTTP Solution (NEB, N0443S)
dUTP Solution (NEB, N0459S)
Primers 1-001, 1-002, 1-003 (Table 1)
AMPure XP beads (Beckman Coulter, A63881)
Takyon™ Rox Probe 5× MasterMix dTTP (Eurogentec, UF-RP5X-C0501)
UDG (NEB, M0372S)
Endo VIII (NEB, M0299S)

### Method

### Linear or PCR Amplification of Target Polynucleotide in the Presence of an Unusual Nucleotide.

A series of difference reaction mixes were prepared as described in the table below.

| | | | PCR | PCR | Linear | Linear |
|---|---|---|---|---|---|---|
| | | | Reac- | Reac- | Reac- | Reac- |
| | | | tion + | tion + | tion + | tion + |
| | | | dTTP | dUTP | dTTP | dUTP |
| Target poly- | 10 | ng/ul | 1 μl | 1 μl | 1 μl | 1 μl |
| nucleotide | | | | | | |
| Vent exo- | 2 | units/μl | 1 μl | 1 μl | 1 μl | 1 μl |
| DNA | | | | | | |
| polymerase | | | | | | |
| Vent exo- | 10x | | 2 μl | 2 μl | 2 μl | 2 μl |
| DNA | | | | | | |
| polymerase | | | | | | |
| buffer | | | | | | |
| dATP | 10 | mM | 1 μl | 1 μl | 1 μl | 1 μl |
| dTTP | 10 | mM | 1 μl | 0 μl | 1 μl | 0 μl |
| dUTP | 10 | mM | 0 μl | 1 μl | 0 μl | 1 μl |
| dCTP | 10 | mM | 1 μl | 1 μl | 1 μl | 1 μl |
| dGTP | 10 | mM | 1 μl | 1 μl | 1 μl | 1 μl |
| 1-001 | 10 | μM | 1 μl | 1 μl | 1 μl | 1 μl |
| 1-002 | 10 | μM | 1 μl | 1 μl | 0 μl | 0 μl |

| | | | | | |
|---|---|---|---|---|---|
| H₂O | — | 11 μl | 11 μl | 11 μl | 11 μl |
| Total volume | | 20 μl | 20 μl | 20 μl | 20 μl |

These mixes were then cycled as follows:

| | | |
|---|---|---|
| Incubation | Incubation | |
| Temperature | Time | Cycles |

| | | | | |
|---|---|---|---|---|
| 95° C. | 1 | min | 1 | cycle |
| 95° C. | 15 | sec | 20 | cycles |
| 60° C. | 1 | min | | |
| 72° C. | 30 | sec | | |
| 72° C. | 2 | min | 1 | cycle |

### Modified First Complementary Strand Digestion.

A 10 μl aliquot of each reaction was taken and to this 0.5 μl of UDG and 0.5 μl Endo VII were added. This mixture was briefly vortexed and centrifuged before being incubated for 20 minutes at 37° C. and 10 minutes at 25° C.

### Bead Purification

To all samples H₂O was added to bring the volume up to 50 μl before being bead purified. The Workflow for the Purification process was as follows:
1. Add the appropriate amount of Ampure beads 100 μl per
2. Pipette mix 10× and incubate at room temperature for 5 mins
3. Place on a magnetic plate for 3 mins and remove supernatant. If beads are disturbed incubate on magnetic plate for a few more minutes
4. Wash beads twice with 150 μl 80% ethanol for 30 seconds each time.
5. Leave tubes uncapped on magnet to dry for 3 mins to remove residual ethanol centrifuge briefly
5. Add 20 μl of H₂O and pipette mix making sure to re-suspend all the beads. Incubate on bench for 2 mins
6. Place back on magnet for approx. 1 mins and retain supernatant
   qPCR Analysis

The following reaction mix was then set up for every bead purified sample.

| | | Volume | |
|---|---|---|---|
| | Concentration | per sample | |
| Bead Purified | NA | 2 | μl |
| Sample | | | |
| Takyon | 5x | 4 | μl |
| Master Mix | | | |
| 1-001 | 10 μM | 0.6 | μl |
| 1-002 | 10 μM | 0.6 | μl |
| 1-003 | 10 μM | 0.4 | μl |
| H₂O | NA | 12.4 | μl |
| Total | | 20 | μl |

The qPCR reaction was thermo cycles as follows.

| | | |
|---|---|---|
| Incubation | Incubation | |
| Temperature | Time | Cycles |

| | | | | |
|---|---|---|---|---|
| 95° C. | 3 | min | 1 | cycle |
| 95° C. | 10 | sec | 43 | cycles |
| 60° C. | 1 | min | | |
| 72° C. | 2 | min | 1 | cycle |

### Results

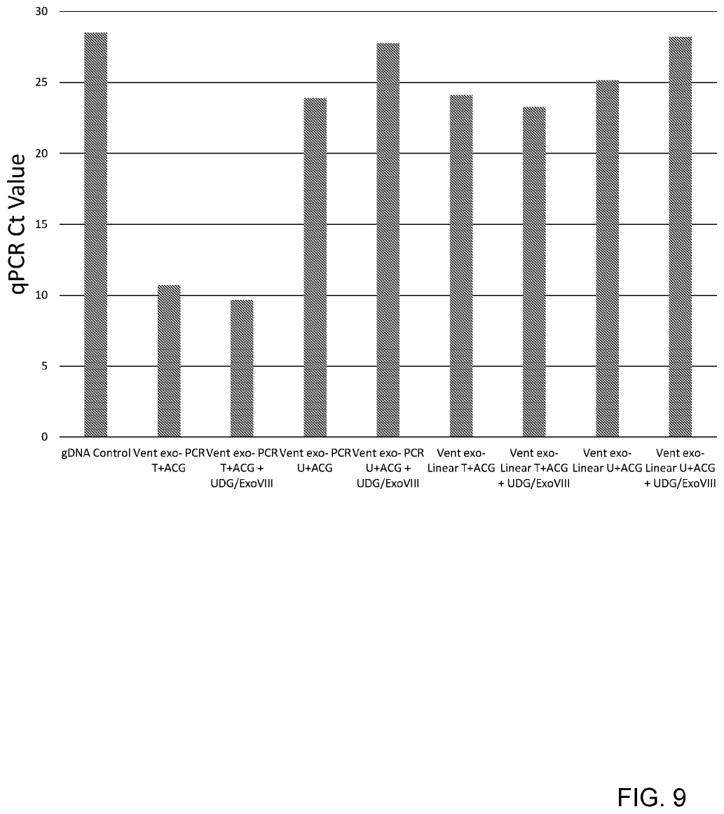

These data (FIG. 9) demonstrate that it is possible for a polymerase to incorporate dUTP into a primer extension product but not be able to efficiently use the extension product as a template. Incorporation is demonstrated by the susceptibility of the linear and PCP amplification products to digestion by UDG and Endo VIII.

### Example 2

Using deoxyribonucleic acid (DNA) as the target polynucleotide for determining the sensitivity of a DNA polymerase to the presence of dU in a reaction mixture to assess the quantity of dU which can be incorporated into a primer extension product while still not being able to use the modified polynucleotide as a template. PCR mixes were prepared using either a single primer, or a pair of opposing primers such that either a linear amplification or exponential amplification would occur in the presence of traditional nucleotides. These reactions were set up with a combination of dATP, dCTP, dGTP, and different ratios of dTTP:dUTP. These reactions were then bead purified and the copy number of the resultant polynucleotides determined by qPCR.

### Materials

Target polynucleotide, human gDNA (ENZ-GEN117-0100)
Vent exo-DNA polymerase (NEB, M0257S)
Vent exo-DNA polymerase buffer (NEB, B9004S)
dATP Solution (NEB, N0440S)
dCTP Solution (NEB, N0441S)
dGTP Solution (NEB, N0442S)
dTTP Solution (NEB, N0443S)
dUTP Solution (NEB, N0459S)
Primers 1-001, 1-002, 1-003 (Table 1)
AMPure XP beads (Beckman Coulter, A63881)
Takyon™ Rox Probe 5× MasterMix dTTP (Eurogentec, UF-RP5X-C0501)

### Method

### Linear or PCR Amplification of Target Polynucleotide in the Presence of an Unusual Nucleotide.

A series of difference reaction mixes were prepared as described in the table below.

| | | | | | | | | | | | | | |
|---|---|---|---|---|---|---|---|---|---|---|---|---|---|
| Target | 10 | 1 | 1 | 1 | 1 | 1 | 1 | 1 | 1 | 1 | 1 | 1 | 1 |
| polynucleotide | ng/ul | μl | μl | μl | μl | μl | μl | μl | μl | μl | μl | μl | μl |
| Vent exo- | 2 | 1 | 1 | 1 | 1 | 1 | 1 | 1 | 1 | 1 | 1 | 1 | 1 |
| DNA | units/μl | μl | μl | μl | μl | μl | μl | μl | μl | μl | μl | μl | μl |
| polymerase | | | | | | | | | | | | | |
| Vent exo- | 10x | 2 | 2 | 2 | 2 | 2 | 2 | 2 | 2 | 2 | 2 | 2 | 2 |
| DNA | | μl | μl | μl | μl | μl | μl | μl | μl | μl | μl | μl | μl |
| polymerase | | | | | | | | | | | | | |
| buffer | | | | | | | | | | | | | |
| dATP | 10 | 1 | 1 | 1 | 1 | 1 | 1 | 1 | 1 | 1 | 1 | 1 | 1 |
| | mM | μl | μl | μl | μl | μl | μl | μl | μl | μl | μl | μl | μl |
| dTTP | 10 | 1 | 0.8 | 0.6 | 0.4 | 0.2 | 0 | 1 | 0.8 | 0.6 | 0.4 | 0.2 | 0 |
| | mM | μl | μl | μl | μl | μl | μl | μl | μl | μl | μl | μl | μl |
| dUTP | 10 | 0 | 0.2 | 0.4 | 0.6 | 0.8 | 1 | 0 | 0.2 | 0.4 | 0.6 | 0.8 | 1 |
| | mM | μl | μl | μl | μl | μl | μl | μl | μl | μl | μl | μl | μl |
| dCTP | 10 | 1 | 1 | 1 | 1 | 1 | 1 | 1 | 1 | 1 | 1 | 1 | 1 |
| | mM | μl | μl | μl | μl | μl | μl | μl | μl | μl | μl | μl | μl |
| dGTP | 10 | 1 | 1 | 1 | 1 | 1 | 1 | 1 | 1 | 1 | 1 | 1 | 1 |
| | mM | μl | μl | μl | μl | μl | μl | μl | μl | μl | μl | μl | μl |
| 1-001 | 10 | 1 | 1 | 1 | 1 | 1 | 1 | 1 | 1 | 1 | 1 | 1 | 1 |
| | μM | μl | μl | μl | μl | μl | μl | μl | μl | μl | μl | μl | μl |
| 1-002 | 10 | 1 | 1 | 1 | 1 | 1 | 1 | 0 | 0 | 0 | 0 | 0 | 0 |
| | μM | μl | μl | μl | μl | μl | μl | μl | μl | μl | μl | μl | μl |
| H₂O | | 11 | 11 | 11 | 11 | 11 | 11 | 12 | 12 | 12 | 12 | 12 | 12 |
| | | μl | μl | μl | μl | μl | μl | μl | μl | μl | μl | μl | μl |
| Total | | 20 | 20 | 20 | 20 | 20 | 20 | 20 | 20 | 20 | 20 | 20 | 20 |
| volume | | μl | μl | μl | μl | μl | μl | μl | μl | μl | μl | μl | μl |

These mixes were then cycled as follows:

| | | |
|---|---|---|
| Incubation | Incubation | |
| Temperature | Time | Cycles |

| | | | | |
|---|---|---|---|---|
| 95° C. | 1 | min | 1 | cycle |
| 95° C. | 15 | sec | 20 | cycles |
| 60° C. | 1 | min | | |
| 72° C. | 30 | sec | | |
| 72° C. | 2 | min | 1 | cycle |

### Bead Purification Process

As per example 1.

qPCR Analysis

As per example 1.

### Results

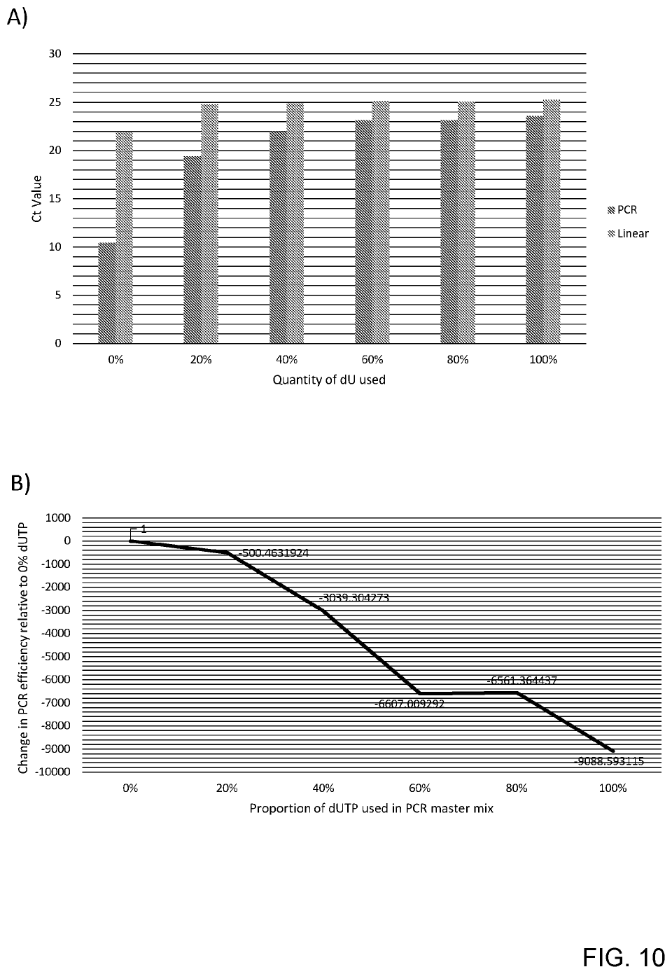

These data (FIG. 10) demonstrate that dU is able to inhibit PCR at low concentrations (0-20%) with the level of inhibition greater than 3-6000× as the concentration reaches 40-60% dU (FIG. 10B). As the proportion of dU reaches close to 100% the level of inhibition also reaches close to and up to 10,000× and the reaction has been converted into a linear amplification reaction as the Ct values converge on the Ct values obtained for the linear amplification reactions.

### Example 3

Using deoxyribonucleic acid (DNA) as the target polynucleotide for generating a high complexity next generation sequencing library using opposing linear amplification primers in the presence or absence of dU to determining the inhibition of PCR.

### Materials

Target polynucleotide, human gDNA (ENZ-GEN117-0100)
Vent exo-DNA polymerase (NEB, M0257S)
Vent exo-DNA polymerase buffer (NEB, B9004S)
dATP Solution (NEB, N0440S)
dCTP Solution (NEB, N0441S)
dGTP Solution (NEB, N0442S)
dTTP Solution (NEB, N0443S)
dUTP Solution (NEB, N0459S)
Primers, 1-004, 1-005, 1-006, 1-007, 1-008 (Table 1)
AMPure XP beads (Beckman Coulter, A63881)
Q5U master mix (NEB, M0597S)
Phusion master mix (Thermofisher, F565S)

### Method

### Linear Amplification of Target Polynucleotide in the Presence of an Unusual Nucleotide.

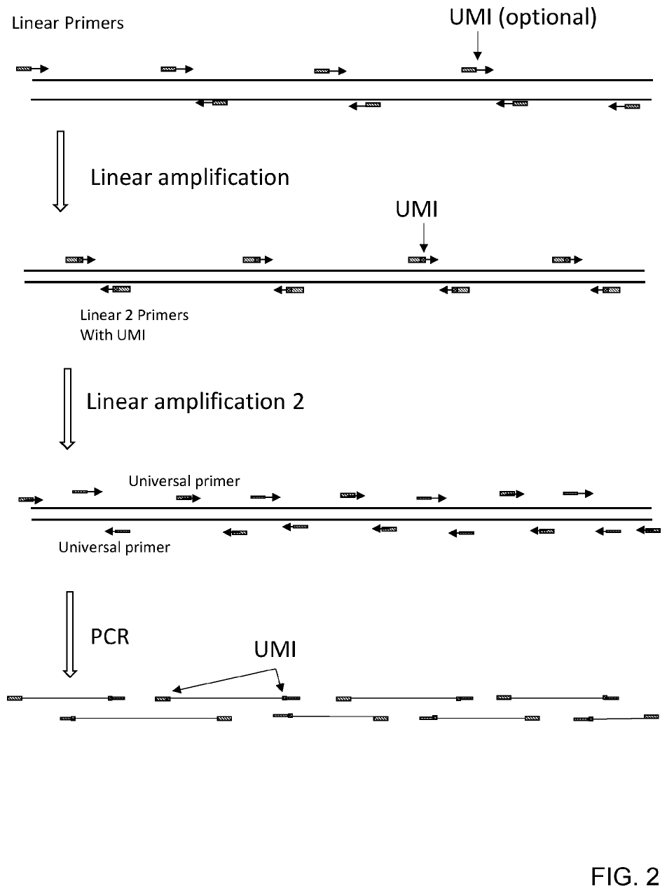

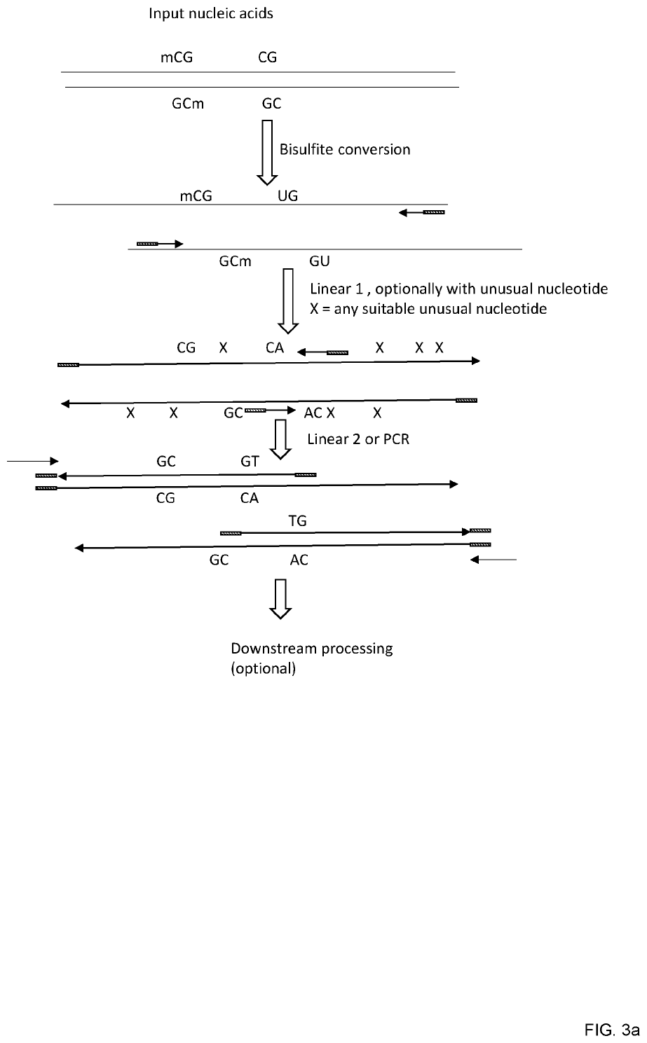

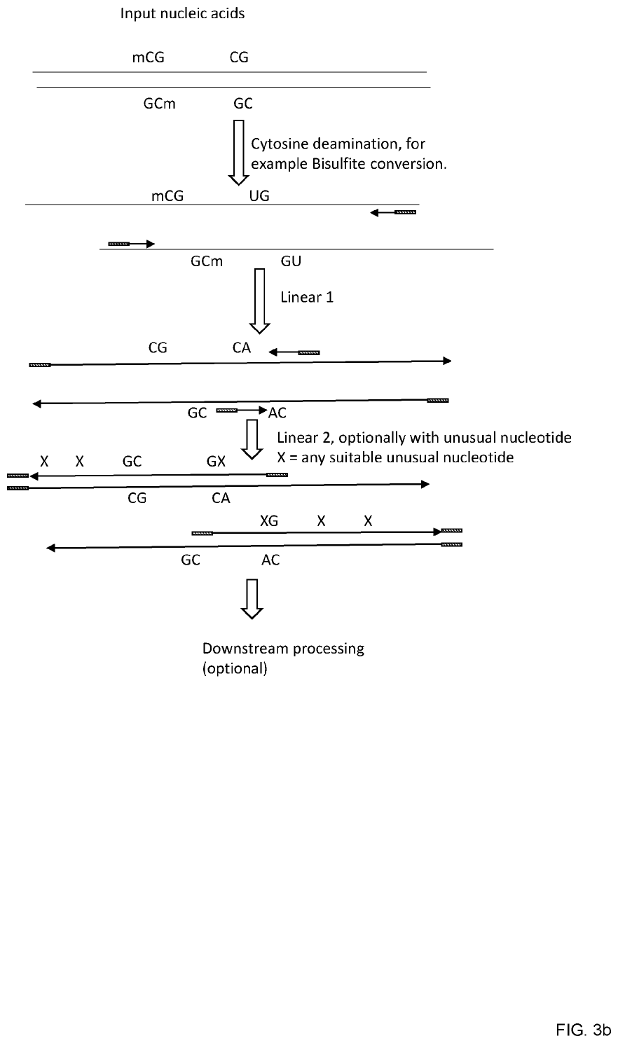

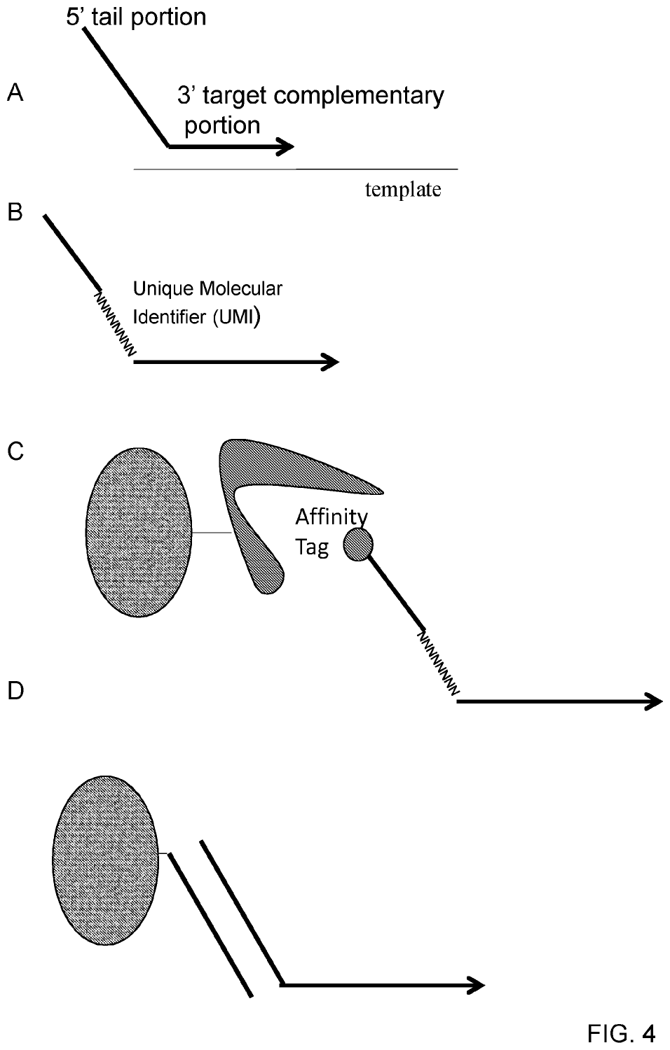

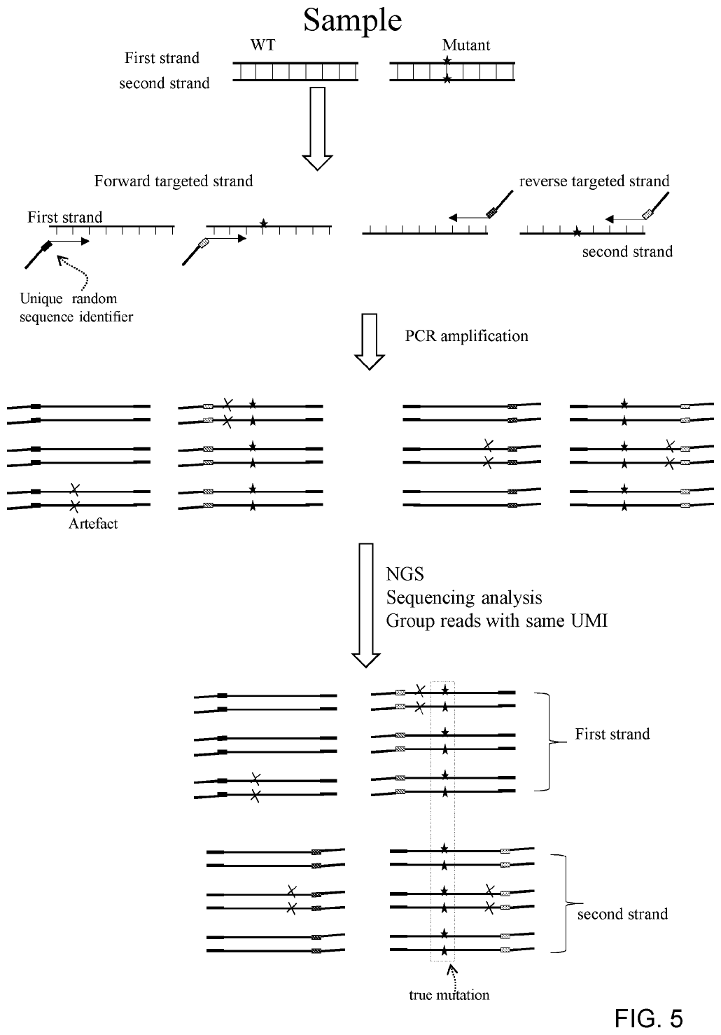

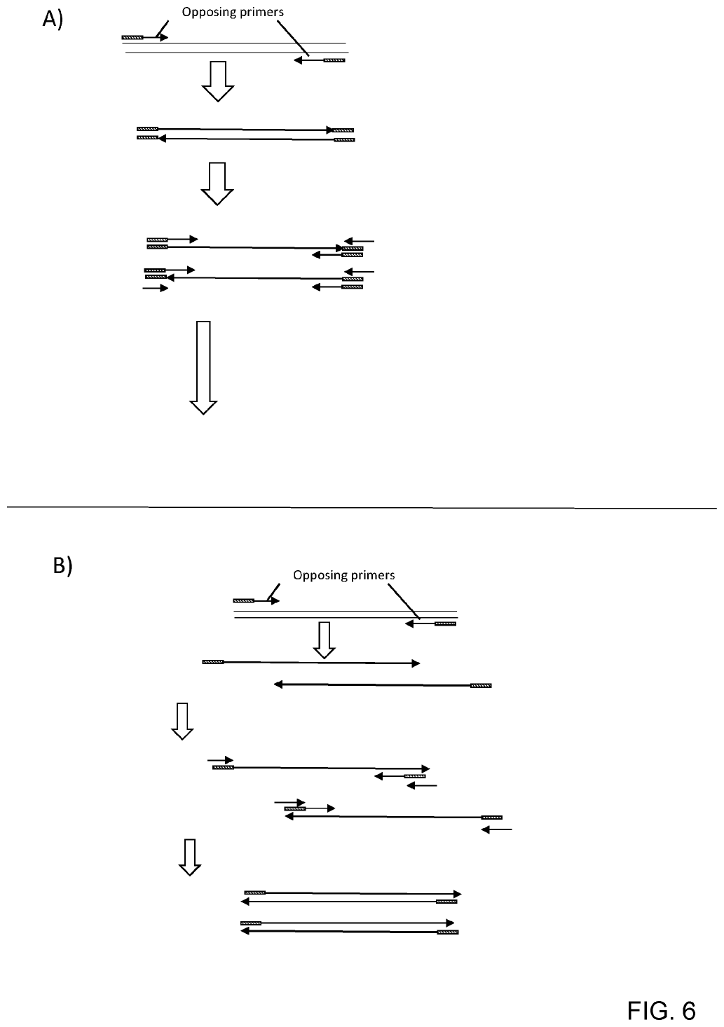

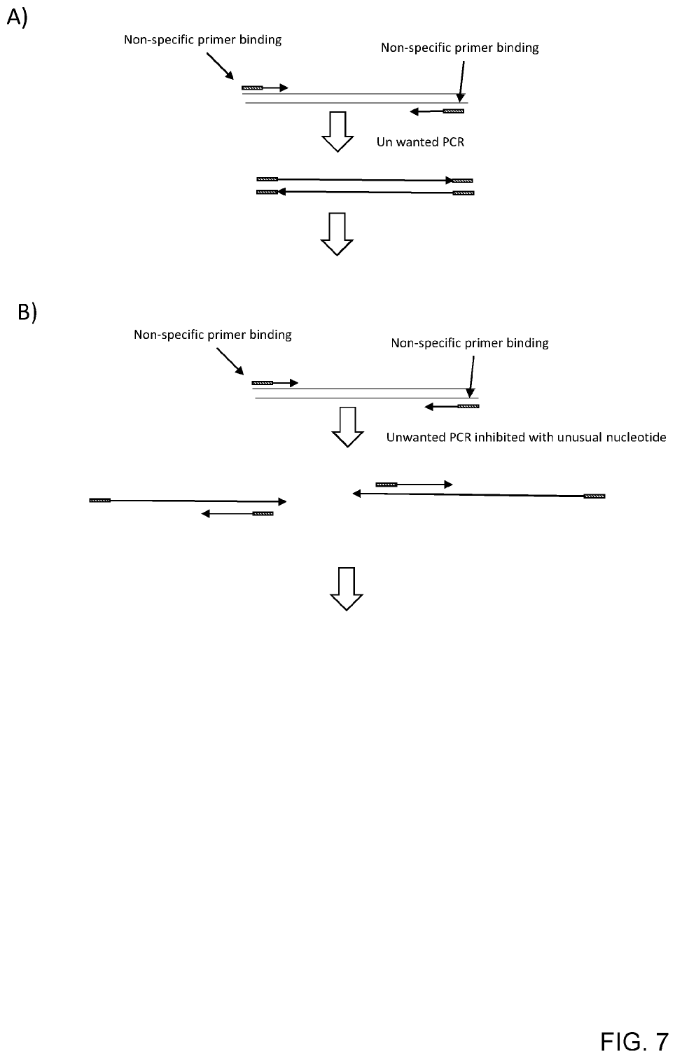

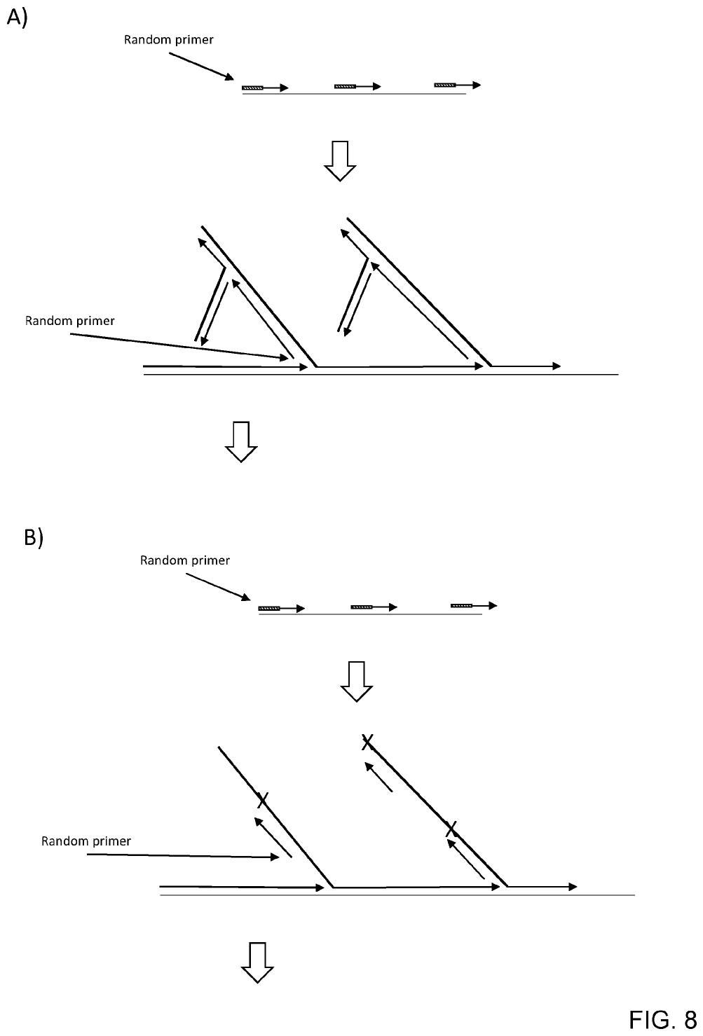

A pool of target specific primers were designed to target 110 frequently mutated hotspots in solid cancers, for selected regions the linear amplification primers were designed flanking the region complementary to the first or second strand so that they were capable of exponential PCR amplification of the region between the primers but this was designed not to occur by the presence of an unusual nucleotide (FIG. 2). All primers contained an 8 bp UMI between the 3′ target specific region and the 5′ universal tail. The primers were pooled at an equal molar ratio. The following reaction mix was prepared.

| Target polynucleotide | 10 | ng/ul | 1 | μl | 1 | μl |
|---|---|---|---|---|---|---|
| Vent exo- DNA polymerase | 2 | units/μl | 1 | μl | 1 | μl |
| Vent exo- DNA polymerase buffer | 10x | | 5 | μl | 5 | μl |
| dATP | 10 | mM | 1 | μl | 1 | μl |
| dTTP | 10 | mM | 0.8 | μl | 1.0 | μl |
| dUTP | 10 | mM | 0.2 | μl | 0 | μl |
| dCTP | 10 | mM | 1 | μl | 1 | μl |
| dGTP | 10 | mM | 1 | μl | 1 | μl |
| 1-005 | 100 | μM | 1 | μl | 1 | μl |

| | | | | |
|---|---|---|---|---|
| H2O | 38 | μl | 38 | μl |
| Total volume | 50 | μl | 50 | μl |

The mixes were then cycled as follows:

| | | |
|---|---|---|
| Incubation | Incubation | |
| Temperature | Time | Cycles |

| | | | | |
|---|---|---|---|---|
| 95° C. | 1 | min | 1 | cycle |
| 95° C. | 15 | sec | 7 | cycles |
| 60° C. | 3 | min | | |
| 65° C. | 30 | sec | | |
| 65° C. | 2 | min | 1 | cycle |

### Bead Purification

As in example 1.

### PCR Amplification

A second pool of target specific primers were designed to target 110 frequently mutated hotspots in solid cancers, for the selected regions where the linear amplification primers were designed flanking the region the target specific PCR primers were design in the middle of the region in a head to head orientation so each is capable of forming a PCR amplifiable pair of primers with one or the other linear primer (FIG. 2). All primers contained a 3′ target specific region and 5′ universal tail. The primers were pooled at an equal molar ratio. The following reaction mix was prepared for both samples.

| | | | |
|---|---|---|---|
| Bead purified linear amplification | — | 23 | μl |
| product | | | |
| Q5U Master Mix | 2x | 25 | μl |
| 1-004 | 25 μM | 1 | μl |
| 1-006 | 100 μM | 1 | μl |
| | Total volume | 50 | μl |

The mixes were then cycled as follows:

| | | |
|---|---|---|
| Incubation | Incubation | |
| Temperature | Time | Cycles |

| | | | | |
|---|---|---|---|---|
| 95° C. | 1 | min | 1 | cycle |
| 95° C. | 15 | sec | 20 | cycles |
| 60° C. | 3 | min | | |
| 65° C. | 30 | sec | | |
| 65° C. | 2 | min | 1 | cycle |

### Bead Purification

As in example 1.

### Indexing PCR

A final PCR reaction using an i5 indexing primer and an i7 indexing primer which anneal to either the linear amplification primer tail or the PCR primer tail are used to produce a final PCR library suitable for sequencing on an Illumina instrument. The following reaction mix was prepared for both samples.

| | | | |
|---|---|---|---|
| Bead purified PCR | — | 23 | μl |
| amplification product | | | |
| Phusion Master Mix | 2x | 25 | μl |
| 1-007 | 100 μM | 1 | μl |
| 1-008 | 100 μM | 1 | μl |
| | Total volume | 50 | μl |

The mixes were then cycled as follows:

| | | |
|---|---|---|
| Incubation | Incubation | |
| Temperature | Time | Cycles |

| | | | | |
|---|---|---|---|---|
| 95° C. | 1 | min | 1 | cycle |
| 95° C. | 15 | sec | 5 | cycles |
| 60° C. | 30 | sec | | |
| 72° C. | 30 | sec | | |
| 72° C. | 2 | min | 1 | cycle |

### Bead Purification

As in example 1.

### Sequencing and Data Analysis

The final PCR library was sequenced using 150 bp PE sequencing on a MiSeq to a depth of approximately 1,000,000 reads. Reads were mapped to the hg38 genome using BWA, the depth of the mapped reads was then counted for the sample containing dUTP+dTTP and the sample containing only dTTP.

### Results

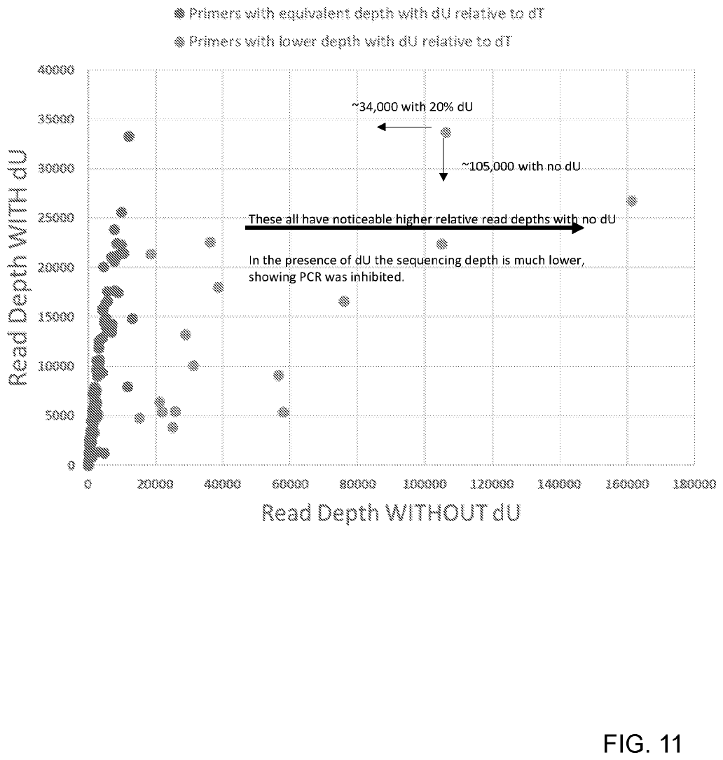

These data demonstrate that in the presence of dU the relative sequencing depth of the sites with opposing primers was significantly lower than the same sites in the presence of dTTP (FIG. 11). This demonstrates the dU can effectively reduce unwanted PCR between two opposing primers and that the method can be incorporated into the generation of a high complexity next generation sequencing library.

### Example 4

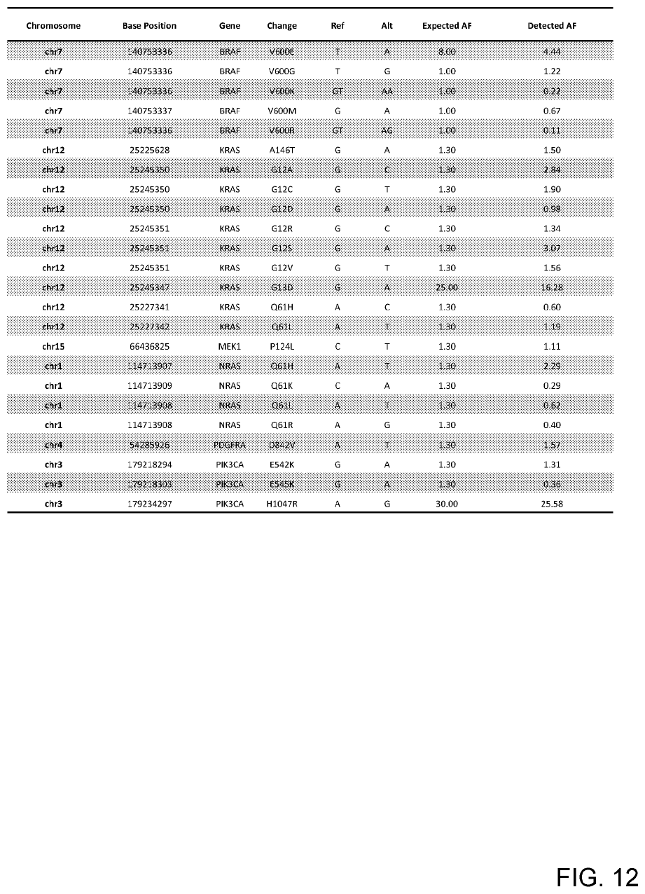

To test a method of the inventions ability to detect mutations from a 1% reference sample the same protocol as example 3 was followed, except a 1% reference sample was used as the target polynucleotide (Horizon discovery, Tru-Q 7 HD734). The final PCR library was sequenced using 150 bp PE sequencing on a MiSeq to a depth of approximately 1,000,000 reads. Reads were mapped to the hg38 genome using BWA, mutations were validated by visualisation in IGV. Examining for the detection of the reference material mutations indicated 100% of the mutations targeted with a target specific primer were identified (FIG. 12).

### Example 5

Using deoxyribonucleic acid (DNA) as the target polynucleotide for generating a high complexity next generation sequencing library using opposing linear amplification primers in the presence of one unusual nucleotide 5-methyl-dCTP, or two unusual nucleotides, 5-methyl-dCTP and dUTP, to generate modified complementary strands which cannot be copied by the polymerase which generated it which is also protected against deamination of cytosine to uracil. Followed by a global deamination of cytosine step and finally targeted amplification of both the original deaminated target polynucleotide and the modified first complementary strand to allow for targeted enrichment of both DNA mutations, and, DNA epigenetic changes.

### Materials

Target polynucleotide, human gDNA (ENZ-GEN117-0100)
Vent exo-DNA polymerase (NEB, M0257S)
Vent exo-DNA polymerase buffer (NEB, B9004S)
dATP Solution (NEB, N0440S)
5-methyl-dCTP Solution (NEB, N0356)
dGTP Solution (NEB, N0442S)
dTTP Solution (NEB, N0443S)
dUTP Solution (NEB, N0459S)
Primers, 1-004, 1-007, 1-008, 1-009, 1-010, 1-011 (Table 1)
AMPure XP beads (Beckman Coulter, A63881)
Q5U master mix (NEB, M0597S)
Phusion master mix (Thermofisher, F565S)
EZ DNA Methylation-Gold (Zymo Research, D5005)

### Method

### First Linear Amplification of Target Polynucleotide in the Presence of an Unusual Nucleotide.

This follows the method of example 3. With the change of using a larger mass of target polynucleotide and using 5-methyl-dCTP in place of dCTP in the reaction mix

| | | | | |
|---|---|---|---|---|
| Target polynucleotide | 10 | ng/ul | 5 | μl |
| Vent exo- DNA polymerase | 2 | units/μl | 1 | μl |

| | | | |
|---|---|---|---|
| Vent exo- DNA polymerase buffer | 10x | 2 | μl |

| | | | | |
|---|---|---|---|---|
| dATP | 10 | mM | 1 | μl |
| dTTP | 10 | mM | 0.8 | μl |
| dUTP or without dUTP | 10 | mM | 0.2 | μl |
| 5-methyl-dCTP | 10 | mM | 1 | μl |
| dGTP | 10 | mM | 1 | μl |
| 1-009 | 100 | μM | 1 | μl |

| | | | |
|---|---|---|---|
| H2O | NA | 7 | μl |
| Total volume | | 20 | μl |

The above reaction mix was thermocycled as per example 3.

### Deamination by a Bisulfite Conversion

The whole of the sample from the previous step is used the conversion process which follow the manufacturer's recommended protocol and the sample is eluted in 25 μl.

### Second Linear Amplification of Converted Target Polynucleotide.

A pool of target specific primers (1-010) was designed to target 50 regions identified as frequently epigenetically altered in solid cancers, and 110 primers designed to amplify opposing the primers 1-009. All primers contained an 8 bp UMI between the 3′ target specific region and the 5′ universal tail. The primers were pooled at an equal molar ratio. The following reaction mix was prepared.

| | | | |
|---|---|---|---|
| Conversion elution product | — | 24 | μl |
| Q5U Master Mix | 2x | 25 | μl |
| 1-010 | 100 μM | 1 | μl |
| | Total volume | 50 | μl |

### Bead Purification

As in example 1.

### PCR Amplification

A second pool of target specific primers were designed to target opposing primers 1-010. All primers contained a 3′ target specific region and 5′ universal tail. The primers were pooled at an equal molar ratio. The following reaction mix was prepared for both samples.

| | | |
|---|---|---|
| Incubation Temperature | Incubation Time | Cycles |

| | | | | |
|---|---|---|---|---|
| 95° C. | 1 | min | 1 | cycle |
| 95° C. | 15 | sec | 20 | cycles |
| 60° C. | 3 | min | | |
| 65° C. | 30 | sec | | |

The mix was then cycled as follows:

| | | | |
|---|---|---|---|
| Bead purified second linear amplification product | — | 23 | μl |
| Q5U Master Mix | 2x | 25 | μl |
| 1-004 | 25 μM | 1 | μl |
| 1-011 | 100 μM | 1 | μl |
| | Total volume | 50 | μl |

| | | |
|---|---|---|
| Incubation | Incubation | |
| Temperature | Time | Cycles |

| | | | | |
|---|---|---|---|---|
| 95° C. | 1 | min | 1 | cycle |
| 95° C. | 15 | sec | 20 | cycles |
| 60° C. | 3 | min | | |
| 65° C. | 30 | sec | | |
| 65° C. | 2 | min | 1 | cycle |

### Bead Purification

As in example 1.

### Indexing PCR

A final PCR reaction using an i5 indexing primer and an i7 indexing primer which anneal to either the linear amplification primer tail or the PCR primer tail are used to produce a final PCR library suitable for sequencing on an Illumina instrument. The following reaction mix was prepared for both samples.

| | | | |
|---|---|---|---|
| Bead purified PCR amplification product | — | 23 | μl |
| Phusion Master Mix | 2x | 25 | μl |
| 1-007 | 100 μM | 1 | μl |
| 1-008 | 100 μM | 1 | μl |
| | Total volume | 50 | μl |

The mixes were then cycled as follows:

| | | |
|---|---|---|
| Incubation Temperature | Incubation Time | Cycles |

| | | | | |
|---|---|---|---|---|
| 95° C. | 1 | min | 1 | cycle |
| 95° C. | 15 | sec | 5 | cycles |
| 60° C. | 30 | sec | | |
| 72° C. | 30 | sec | | |
| 72° C. | 2 | min | 1 | cycle |

### Bead Purification

As in example 1.

### Results

This example demonstrates a method to obtain genetic information from a target polynucleotide with a step that generates a modified complementary strand using an unusual nucleotide which is protected from deamination, follow by a deamination step which converts only the original target polynucleotide. These two populations of polynucleotide can then selectively amplified and used to extract genetic and epigenetic information from a single sample without having to try and extract mutation information from a polynucleotide which has undergone a deamination processes. Where after deamination a linear amplification step allow for all amplification products to contain UMIs.

### Example 6

Using deoxyribonucleic acid (DNA) as the target polynucleotide for generating a high complexity next generation sequencing library using opposing linear amplification primers in the presence of one unusual nucleotide 5-methyl-dCTP, alternatively two unusual nucleotides, 5-methyl-dCTP and dUTP, to generate modified complementary strands which cannot be copied by the polymerase which generated it which is also protected against deamination of cytosine to uracil. Followed by a global deamination of cytosine step and finally targeted amplification of both the deaminated original target polynucleotide and the modified first complementary strand to allow for targeted enrichment of both DNA base mutations, and, DNA epigenetic changes.

### Materials

Target polynucleotide, human gDNA (ENZ-GEN117-0100)
Vent exo-DNA polymerase (NEB, M0257S)
Vent exo-DNA polymerase buffer (NEB, B9004S)
dATP Solution (NEB, N0440S)
5-methyl-dCTP Solution (NEB, N0356)
dGTP Solution (NEB, N0442S)
dTTP Solution (NEB, N0443S)
dUTP Solution (NEB, N0459S)
Primers, 1-004, 1-007, 1-008, 1-009, 1-011 (Table 1)
AMPure XP beads (Beckman Coulter, A63881)
Q5U master mix (NEB, M0597S)
Phusion master mix (Thermofisher, F565S)
EZ DNA Methylation-Gold (Zymo Research, D5005)

### Method

### First Linear Amplification of Target Polynucleotide in the Presence of an Unusual Nucleotide.

As in example 5.

### Deamination by a Bisulfite Conversion

As in example 5.

### PCR Amplification

A second pool of target specific primers were designed to target opposing primers 1-010. All primers contained a 3′ target specific region and 5′ universal tail. The primers were pooled at an equal molar ratio. The following reaction mix was prepared for both samples.

| | | | |
|---|---|---|---|
| Bead purified second linear amplification product | — | 23 | μl |
| Q5U Master Mix | 2x | 25 | μl |
| 1-004 | 25 μM | 1 | μl |
| 1-011 | 100 μM | 1 | μl |
| | Total volume | 50 | μl |

The mix was then cycled as follows:

| | | |
|---|---|---|
| Incubation | Incubation | |
| Temperature | Time | Cycles |

| | | | | |
|---|---|---|---|---|
| 95° C. | 1 | min | 1 | cycle |
| 95° C. | 15 | sec | 20 | cycles |
| 60° C. | 3 | min | | |
| 65° C. | 30 | sec | | |
| 65° C. | 2 | min | 1 | cycle |

### Bead Purification

As in example 1.

### Indexing PCR

A final PCR reaction using an i5 indexing primer and an i7 indexing primer which anneal to either the linear amplification primer tail or the PCR primer tail are used to produce a final PCR library suitable for sequencing on an Illumina instrument. The following reaction mix was prepared for both samples.

| | | | |
|---|---|---|---|
| Bead purified PCR amplification product | — | 23 | μl |
| Phusion Master Mix | 2x | 25 | μl |
| 1-007 | 100 μM | 1 | μl |
| 1-008 | 100 μM | 1 | μl |
| | Total volume | 50 | μl |

The mixes were then cycled as follows:

| | | |
|---|---|---|
| Incubation Temperature | Incubation Time | Cycles |

| | | | | |
|---|---|---|---|---|
| 95° C. | 1 | min | 1 | cycle |
| 95° C. | 15 | sec | 5 | cycles |
| 60° C. | 30 | sec | | |
| 72° C. | 30 | sec | | |
| 72° C. | 2 | min | 1 | cycle |

### Bead Purification

As in example 1.

### Results

This example demonstrates a second method of the embodiment of the invention that obtains genetic information by the generation of copies of a target polynucleotide producing modified complementary strands using an unusual nucleotide which protects the modified complementary strand from deamination, follow by a deamination step which is only able to convert unmodified cytosine present in the original target polynucleotide. Using fewer amplification steps than example 5 these two populations of polynucleotide are then be used to extract genetic and epigenetic information from a single original population of polynucleotide.

### Example 7

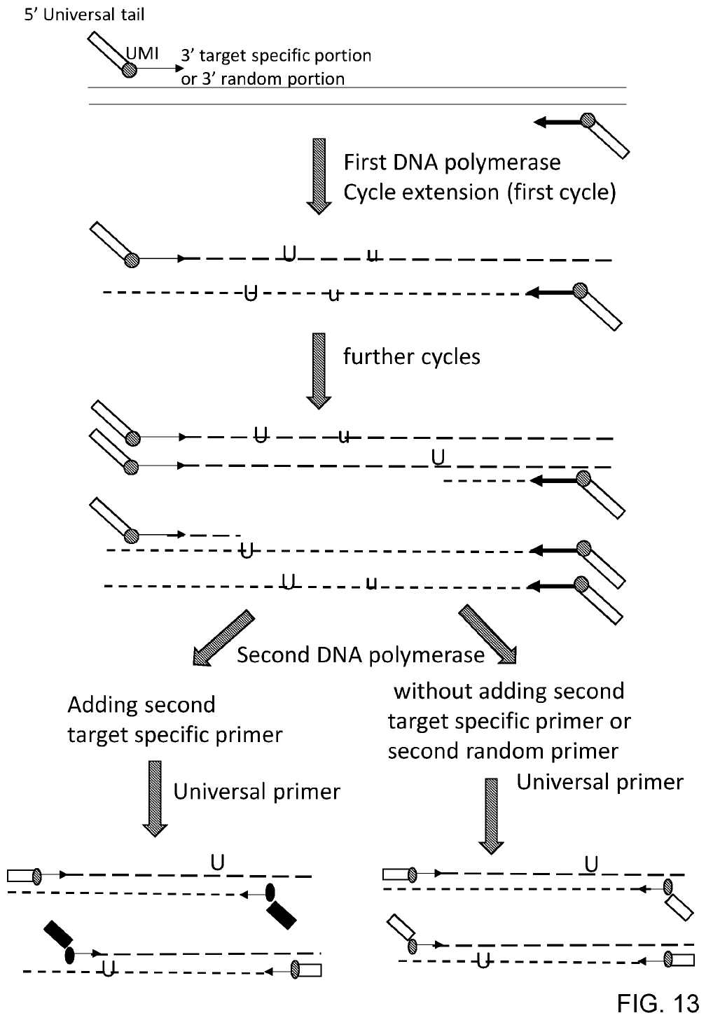

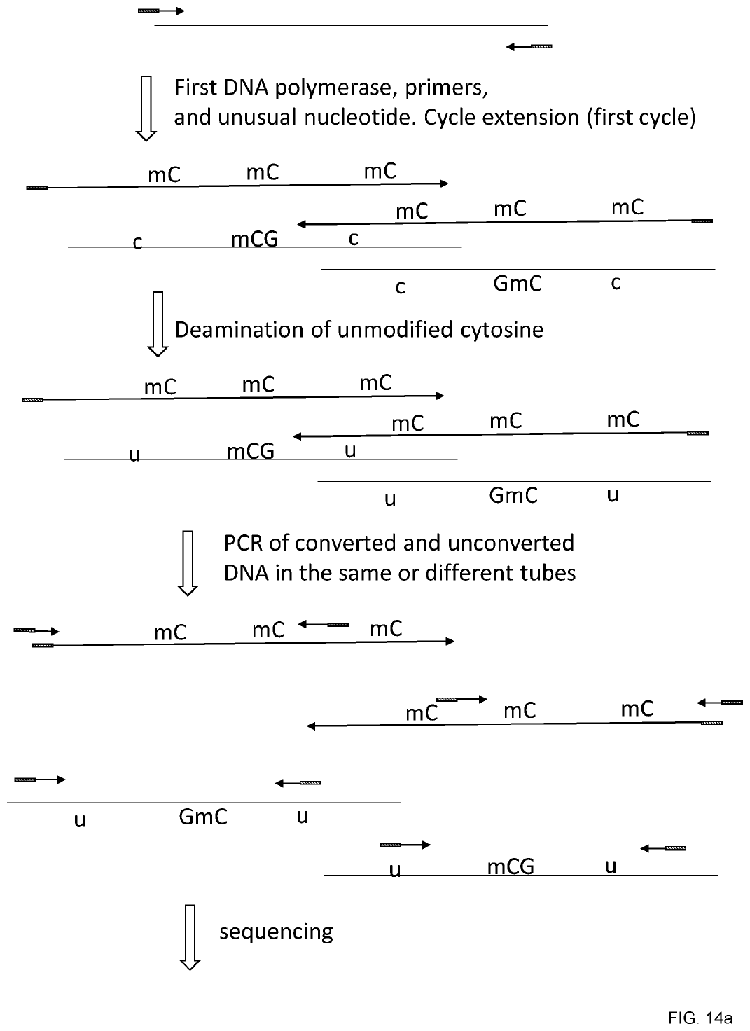

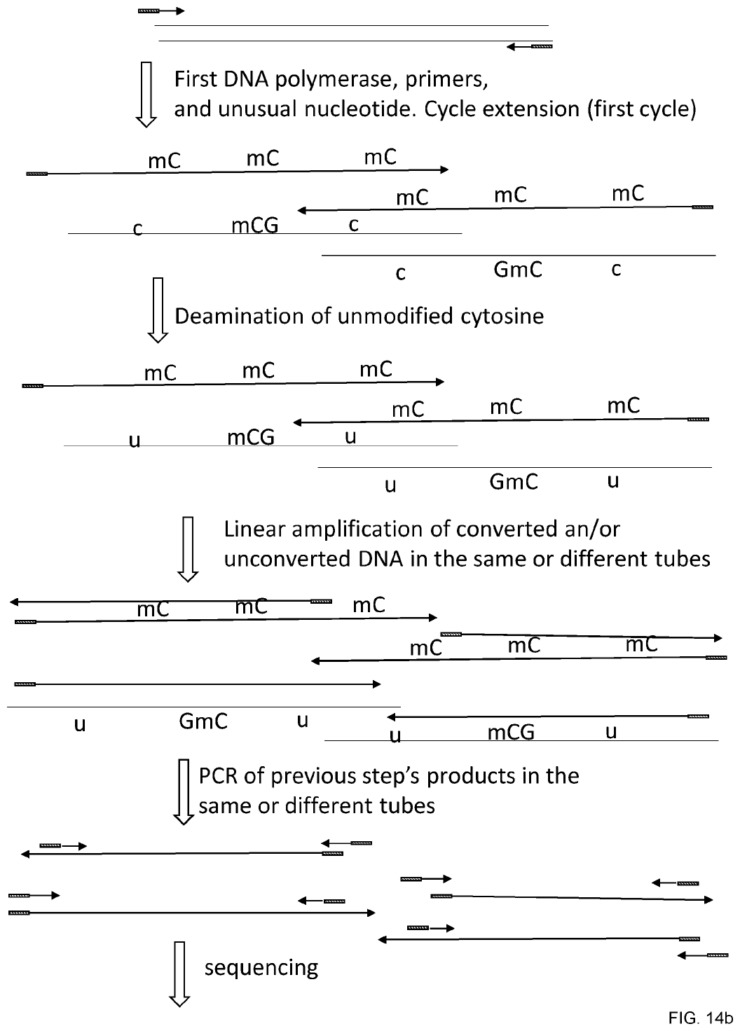

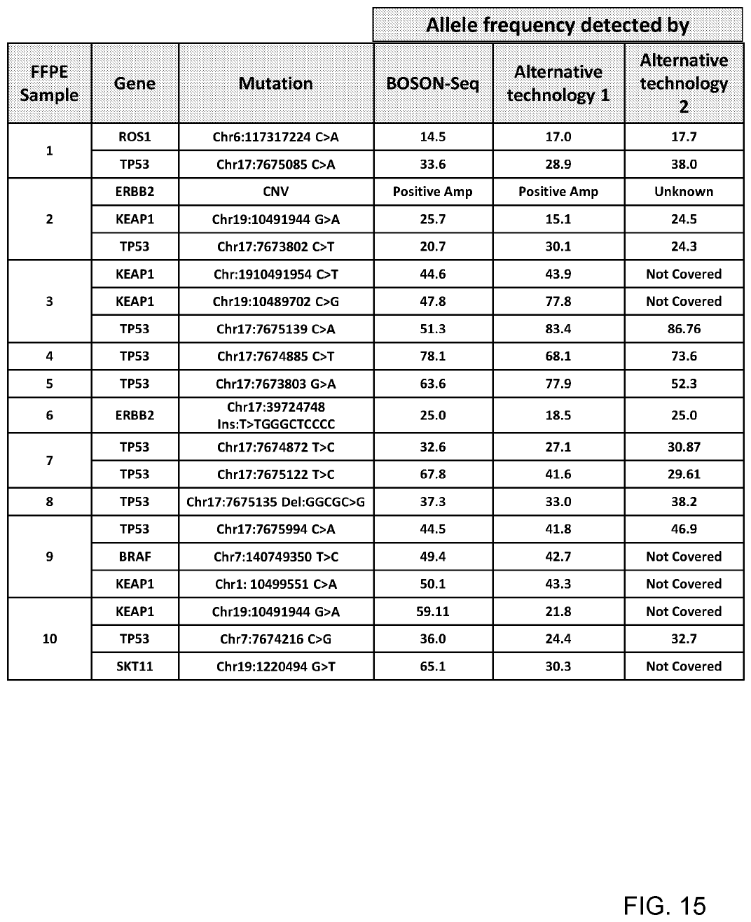

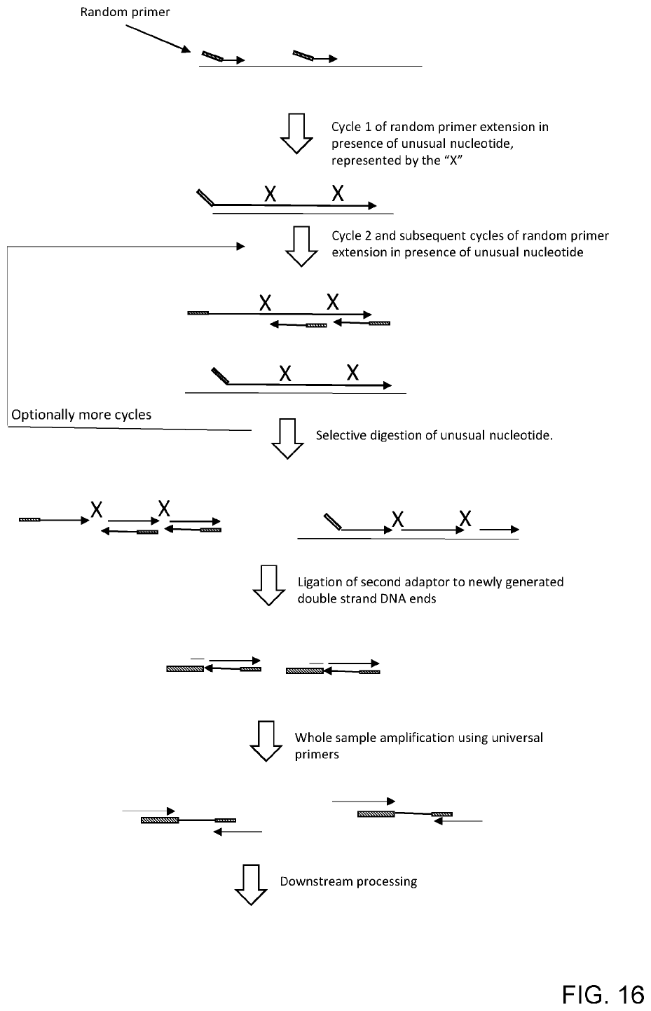

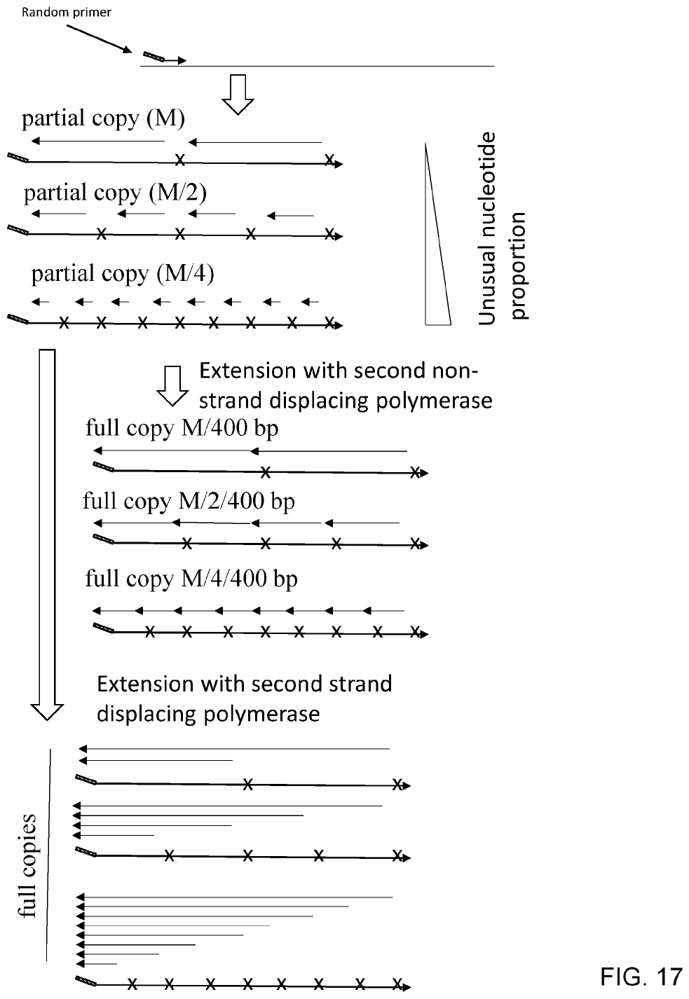

Using deoxyribonucleic acid (DNA) as the target polynucleotide for generating a high complexity next generation sequencing library using random primers in the presence of an unusual nucleotide, dUTP, to initially generate whole genome amplified modified complementary strands which cannot be efficiently copied by the polymerase which generated them to reduce the bias in the whole genome amplification. Followed by additional amplification to generate a next generation sequencing ready sequencing library as a representation of the original target polynucleotide. See, in some cases, FIG. 13 for a schematic representation of this example.

### Materials

Target polynucleotide, human gDNA (ENZ-GEN117-0100)
Vent exo-DNA polymerase (NEB, M0257S)
Vent exo-DNA polymerase buffer (NEB, B9004S)
dATP Solution (NEB, N0440S)
dGTP Solution (NEB, N0442S)
dTTP Solution (NEB, N0443S)
dCTP Solution (NEB, N0441S)
dUTP Solution (NEB, N0459S)
Primers, 1-012, 1-013. 1-007, 1-008 (Table 1)
AMPure XP beads (Beckman Coulter, A63881)
Q5U master mix (NEB, M0597S)
Phusion master mix (Thermofisher, F565S)

### First Linear Amplification of Target Polynucleotide in the Presence of an Unusual Nucleotide.

A primer with a 3′ random sequence in the presence of an unusual nucleotide to inhibit or otherwise suppress the exponential amplification of DNA. The following reaction mix was prepared.

| | | | | |
|---|---|---|---|---|
| Target polynucleotide | 50 | ng/μl | 1 | μl |
| Vent exo- DNA polymerase | 2 | units/μl | 1 | μl |

| | | | |
|---|---|---|---|
| Vent exo- DNA polymerase | 10x | 5 | μl |
| buffer | | | |

| | | | | |
|---|---|---|---|---|
| dATP | 10 | mM | 1 | μl |
| dTTP | 10 | mM | 0.99 | μl |
| dUTP | 1 | mM | 1.0 | μl |
| dCTP | 10 | mM | 1 | μl |
| dGTP | 10 | mM | 1 | μl |
| 1-012 | 100 | μM | 1 | μl |
| 1-013 | 100 | μM | 1 | μl |

| | | |
|---|---|---|
| H2O | 36.01 | μl |
| Total volume | 50 | μl |

The mixes were then cycled as follows:

| | | |
|---|---|---|
| Incubation | Incubation | |
| Temperature | Time | Cycles |

| | | | |
|---|---|---|---|
| 95° | C. | 1 min | 1 |
| 95° | C. | 1 min | |
| 16-60° | C. | 5 min | 3 |
| 72° | C. | 5 min | |

### Bead Purification

As in example 1.

### Whole Sample Amplification

A final PCR amplification reaction using an i5 indexing primer and an i7 indexing primer are used to produce a final PCR library suitable for sequencing on an Illumina instrument. The following reaction mix was prepared.

| | | | |
|---|---|---|---|
| Bead purified product | — | 23 | μl |
| Q5U master mix | 2x | 25 | μl |
| 1-007 | 100 μM | 1 | μl |
| 1-008 | 100 μM | 1 | μl |
| | Total volume | 50 | μl |

The mixes were then cycled as follows:

| | | |
|---|---|---|
| Incubation Temperature | Incubation Time | Cycles |

| | | | | |
|---|---|---|---|---|
| 50-65° | C. | 5 | min | 1 |
| 95° | C. | 1 | min | 1 |
| 95° | C. | 15 | sec | 20 |
| 60° | C. | 30 | sec | |
| 65° | C. | 30 | sec | |
| 65° | C. | 2 | min | 1 |

### Bead Purification

As in example 1.

### Results

This example demonstrates an embodiment of the invention in which the entire population of a polynucleotide can be amplified in a way that reduces amplification bias giving more uniform coverage of the input.

### Example 8

To test a method of the inventions ability to detect mutations from a clinical sample the same protocol as example 3 was followed, except 10 different lung cancer FFPE samples were used as the target polynucleotide. The final PCR libraries were sequenced using 150 bp PE sequencing on a MiSeq to a depth of approximately 1,000,000 reads. Reads were mapped to the hg38 genome using BWA, mutations were validated by visualisation in IGV. All samples had previously been screened for mutations using an alternative technology. Examining for the detection of the expected FFPE mutations indicated 100% of the mutations targeted with a target specific primer were identified).

### Example 9

Using deoxyribonucleic acid (DNA) as the target polynucleotide for generating a high complexity next generation sequencing library using random primers in the presence of an unusual nucleotide, dUTP, to initially generate whole genome amplified modified complementary strands which cannot be efficiently copied by the polymerase which generated them to reduce the bias in the whole genome amplification. Followed by digestion at the incorporation positions of the unusual nucleotide. Followed by ligation of adaptors to generate a second universal primer site. Followed by additional amplification to generate a next generation sequencing ready sequencing library as a representation of the original target polynucleotide. See, in some cases, FIG. 16 for a schematic representation of this example.

### Materials

Target polynucleotide, human gDNA (ENZ-GEN117-0100)
Vent exo-DNA polymerase (NEB, M0257S)
Vent exo-DNA polymerase buffer (NEB, B9004S)
dATP Solution (NEB, N0440S)
dGTP Solution (NEB, N0442S)
dTTP Solution (NEB, N0443S)
dCTP Solution (NEB, N0441S)
dUTP Solution (NEB, N0459S)
Primers, 1-007, 1-008, 1-014, 1-015, 1-016 (Table 1)
AMPure XP beads (Beckman Coulter, A63881)
Q5U master mix (NEB, M0597S)
UDG (NEB, M0280S)
Exo VIII (NEB, M0299S)
NEBNext® Quick Ligation Module (NEB, E6056S)
NEBNext End Prep (NEB, E7442)

### First Linear Amplification of Target Polynucleotide in the Presence of an Unusual Nucleotide.

A primer with a 3′ random sequence in the presence of an unusual nucleotide to inhibit or otherwise suppress the exponential amplification of DNA. The following reaction mix was prepared.

| | | | | |
|---|---|---|---|---|
| Target polynucleotide | 50 | ng/μl | 1 | μl |
| Vent exo- DNA polymerase | 2 | units/μl | 1 | μl |

| | | | |
|---|---|---|---|
| Vent exo- DNA polymerase buffer | 10x | 5 | μl |

| | | | | |
|---|---|---|---|---|
| dATP | 10 | mM | 1 | μl |
| dTTP | 10 | mM | 0.99 | μl |
| dUTP | 0.1 | mM | 1 | μl |
| dCTP | 10 | mM | 1 | μl |
| dGTP | 10 | mM | 1 | μl |
| 1-014 | 100 | μM | 1 | μl |

| | | |
|---|---|---|
| H2O | 37.01 | μl |
| Total volume | 50 | μl |

The mixes were then cycled as follows:

| | | |
|---|---|---|
| Incubation | Incubation | |
| Temperature | Time | Cycles |

| | | | |
|---|---|---|---|
| 95° | C. | 1 min | 1 |
| 95° | C. | 1 min | 3 |
| 16-60° | C. | 5 min | |
| 72° | C. | 5 min | |

### Bead Purification

As in example 1.

### Digestion of Unusual Nucleotide

The following reaction mix was prepared.

| | | | |
|---|---|---|---|
| Purified sample | — | 16 | μl |
| NEB buffer 2 | 10 x | 2 | μl |

| | | | | |
|---|---|---|---|---|
| UDG | 5,000 | units/ml | 1 | μl |
| Exo VIII | 10,000 | units/ml | 1 | μl |

| | | |
|---|---|---|
| Total volume | 20 | μl |

The mix was then cycled as follows:

| Incubation | Incubation | |
|---|---|---|
| Temperature | Time | Cycles |
| 37° C. | 30 min | 1 |

### End Repair and Ligation of Adaptors.

The following reaction mix was prepared.

| | | | |
|---|---|---|---|
| Sample | — | 20 | μl |
| End Prep Enzyme Mix | 10 x | 1 | μl |
| End Repair Reaction Buffer | — | 3 | μl |
| H₂O | — | 6 | μl |
| Total volume | | 30 | μl |

The mix was then cycled as follows:

| Incubation | Incubation | |
|---|---|---|
| Temperature | Time | Cycles |
| 20° C. | 30 min | 1 |
| 65° C. | 30 min | 1 |

The following oligos were mixed together.

| | | | |
|---|---|---|---|
| 1-015 | 100 μM | 1.5 | μl |
| 1-016 | 100 μM | 1.5 | μl |
| Lol TE buffer | — | 97 | μl |

The mix was then cycled as follows:

| Incubation Temperature | Incubation Time | Cycles |
|---|---|---|
| 95° C. | 5 min | 1 |
| Gradient from 95-10° C. | 30 seconds/1° C. | 1 |

The following reaction mix was prepared and directly added to the above sample.

| | | | |
|---|---|---|---|
| Adaptor | 1.5 μM | 0.75 | μl |
| Ligation Enhancer | — | 0.25 | μl |
| Blunt/TA Ligase Master Mix | — | 7 | μl |
| Total volume | | 38 | μl |

The mix was then cycled as follows:

| Incubation | Incubation | |
|---|---|---|
| Temperature | Time | Cycles |
| 20° C. | 15 min | 1 |

### PCR Amplification Adaptors.

The following reaction mix was prepared and directly added to the above sample.

| | | | |
|---|---|---|---|
| Q5U master mix | 2x | 40 | μl |
| 1-007 | 50 μM | 2 | μl |
| 1-008 | 50 μM | 2 | μl |
| Previous steps product | — | 38 | μl |

The mix was then cycled as follows:

| | | |
|---|---|---|
| Incubation Temperature | Incubation Time | Cycles |

| | | | | |
|---|---|---|---|---|
| 95° C. | 1 | min | 1 | cycle |
| 95° C. | 15 | sec | 20 | cycles |
| 60° C. | 30 | sec | | |
| 65° C. | 30 | sec | | |
| 72° C. | 2 | min | 1 | cycle |

### Bead Purification

As in example 1.

### Results

This example demonstrates an embodiment of the invention that obtains genetic and epigenetic information from a single sample without a deamination step by sodium bisulfite confusing mutations which could be confused by deamination of C.

### Example 10

Using deoxyribonucleic acid (DNA) as the target polynucleotide for generating a high complexity next generation sequencing library using random primers in the presence of an unusual nucleotide, dUTP, to initially generate whole genome amplified modified complementary strands which cannot be efficiently copied by the polymerase which generated them to reduce the bias in the whole genome amplification with different proportions of dU to demonstrate that both molar number of copies and/or length of the copies can be modulated by adjusting the proportion of dU. Followed by additional amplification to generate a next generation sequencing ready sequencing library as a representation of the original target polynucleotide. See, in some cases, FIG. 17 for a schematic representation of this example.

### Materials

Target polynucleotide, human gDNA (ENZ-GEN117-0100)
Vent exo-DNA polymerase (NEB, M0257S)
Vent exo-DNA polymerase buffer (NEB, B9004S)
dATP Solution (NEB, N0440S)
dGTP Solution (NEB, N0442S)
dTTP Solution (NEB, N0443S)
dCTP Solution (NEB, N0441S)
dUTP Solution (NEB, N0459S)
Primers, 1-007, 1-008, 1-014, 1-015, 1-016 (Table 1)
AMPure XP beads (Beckman Coulter, A63881)
Q5U master mix (NEB, M0597S)
Klenow exo-(NEB, M0212S)

### First Linear Amplification of Target Polynucleotide in the Presence of an Unusual Nucleotide.

A primer with a 3′ random sequence in the presence of an unusual nucleotide to inhibit or otherwise suppress the exponential amplification of DNA. The following reaction mix was prepared.

| | | | Volume (μl) | | | | | |
|---|---|---|---|---|---|---|---|---|
| | Sample | | 1 | 2 | 3 | 4 | 5 | 6 |
| Target | 50 | ng/ul | 1 | 1 | 1 | 1 | 1 | 1 |
| poly- | | | | | | | | |
| nucleotide | | | | | | | | |
| Vent exo- | 2 | units/μl | 1 | 1 | 1 | 1 | 1 | 1 |
| DNA | | | | | | | | |
| polymerase | | | | | | | | |
| Vent exo- | 10x | | 5 | 5 | 5 | 5 | 5 | 5 |
| DNA | | | | | | | | |
| polymerase | | | | | | | | |
| buffer | | | | | | | | |
| dATP | 10 | mM | 1 | 2 | 3 | 1 | 1 | 1 |
| dTTP | 10 | mM | 0.99 | 0.98 | 0.96 | 0.99 | 0.98 | 0.96 |
| dUTP | 0.1 | mM | 1 | 2 | 4 | 1 | 2 | 4 |
| dCTP | 10 | mM | 1 | 1 | 1 | 1 | 1 | 1 |
| dGTP | 10 | mM | 1 | 1 | 1 | 1 | 1 | 1 |
| 1-014 | 100 | μM | 1 | 1 | 1 | 1 | 1 | 1 |
| H2O | | | 37.01 | 37.02 | 37.04 | 37.01 | 37.02 | 37.04 |
| Total volume | | | 50 | 50 | 50 | 50 | 50 | 50 |

The mixes were then cycled as follows:

| | | |
|---|---|---|
| Incubation | Incubation | |
| Temperature | Time | Cycles |

| | | | |
|---|---|---|---|
| 95° | C. | 1 min | 1 |
| 95° | C. | 1 min | 3 |
| 16-60° | C. | 5 min | |
| 72° | C. | 5 min | |

### Bead Purification

As in example 1.

### Second Extension

The following reaction mixtures were prepared.

| | | | Samples 1-3 | | Samples 4-6 | |
|---|---|---|---|---|---|---|
| Purified sample | — | | 20 | μl | 20 | μl |
| Q5U master mix | 2x | | 0.0 | μl | 25 | μl |
| NEB buffer 2 | 10 x | | 2.5 | μl | 0.0 | μl |
| Klenow exo- | 5,000 | units/ml | 1 | μl | 0.0 | μl |
| dNTPs | 10 | mM | 1 | μl | 0.0 | μl |

| | | | | | |
|---|---|---|---|---|---|
| H₂O | — | 0.5 | μl | 3 | μl |
| Total volume | — | 25 | μl | 48 | μl |

The mixes for the different samples were then cycled as follows:

| Incubation | Incubation | Sample 1-3 |
|---|---|---|
| Temperature | Time | Cycles |
| 37° C. | 15 min | 1 |

| Incubation | Incubation | Sample 4-6 |
|---|---|---|
| Temperature | Time | Cycles |
| 65° C. | 15 min | 1 |

The following reaction mix was prepared and directly added to the above sample.

| | | Samples 1-3 | | Samples 4-6 |
|---|---|---|---|---|
| Q5U master mix | 2x | 25 | μl | 0 μl |
| 1-007 | 50 μM | 1 | μl | 1 μl |
| 1-008 | 50 μM | 1 | μl | 1 μl |
| Previous steps product | — | 23 | μl | 48 μl |

The mix was then cycled as follows:

| | | |
|---|---|---|
| Incubation Temperature | Incubation Time | Cycles |

| | | | | |
|---|---|---|---|---|
| 95° C. | 1 | min | 1 | cycle |
| 95° C. | 15 | sec | 10 | cycles |
| 60° C. | 30 | sec | | |
| 65° C. | 30 | sec | | |
| 65° C. | 2 | min | 1 | cycle |

### Bead Purification

As in example 1.

### Results

This example demonstrates an embodiment of the invention that allow for the adjustment of the size distribution of the finial amplification products as well as adjusting the final molar yields of amplification products by adjust a combination of the percentage of unusual nucleotides and by adjusting the activities of different polymerase at time points in a workflow.

1. A method of processing target nucleic acids comprising
(a) providing a reaction mixture(s), each reaction mixture comprising a first polymerase, one or more unusual nucleoside triphosphates and a first primer, wherein the polymerase is capable of extending a primer using the target nucleic acids as templates and incorporating the unusual nucleotide into extension products to produce modified complementary strands, and cannot efficiently make a further copy using the modified complementary strands as templates, wherein the unusual nucleoside triphosphate is distinct from the four standard nucleotides; and
(b) performing one pass extension or cycles of extension reactions of the first primer on target nucleic acid template to produce modified complementary strands, which cannot efficiently be served as template for further copying in the reaction using the first polymerase.

2. The method of claim 1, comprising
(a) providing a reaction mixture(s), each reaction mixture comprising a first polymerase, four or more different nucleoside triphosphates including one or more unusual nucleoside triphosphates and a first primer, wherein the polymerase is capable of extending a primer using the target nucleic acids as templates and incorporating the unusual nucleotide into extension products to produce modified complementary strands, and is incapable of efficiently making a further copy using the modified complementary strand as template for extension of primers in the opposite orientation, wherein the unusual nucleoside triphosphate is distinct from the four standard nucleotides (deoxyadenosine triphosphate (dATP), deoxythymidine triphosphate (dTTP), deoxyguanosine triphosphate (dGTP), and deoxycytidine triphosphate (dCTP)), and is capable of being incorporated into new strands;
(b) performing one pass extension or cycles of extension reactions of the first primer on target nucleic acid template to produce modified complementary strands,
(c) adding a second polymerase which is capable of using the modified complementary strands as templates; and
(d) replicating or amplifying the modified complementary strands and/or the original strands using the second polymerase.

3. The method of claim 1, wherein the cycles of extension reactions comprise at least two cycles.

4. The method of claim 3, wherein the cycles of extension reactions comprise 2 to 40 cycles.

5. The method of claim 2, wherein step (c) further comprises adding a second primer which is capable of extension in step (d).

6. The method of claim 1, after step (b) further comprising removing the unusual nucleoside triphosphate and/or primers by purification or an enzymatic reaction.

7. The method of claim 1, wherein the unusual nucleoside triphosphate is selected from: ribonucleoside triphosphate, deoxyinosine triphosphate, 2′-O-Methyladenosine-5′-Triphosphate, 2′-O-Methylcytidine-5′-Triphosphate, 2′-O-Methylguanosine-5′-Triphosphate, 2′-O-Methyluridine-5′-Triphosphate, 5-Methyl-2′-deoxycytidine-5′-Triphosphate or 2′-Deoxyuridine-5′-Triphosphate.

8. The method of claim 1, wherein the unusual nucleotide is 5-Methyl-2′-deoxycytidine-5′-Triphosphate, wherein after step (b) the DNA mixture is deaminated by chemical and/or enzymatic processes, wherein the modified complementary strands are protected from deamination, and wherein the original strands are deaminated on the sites not methylated.

9. The method of claim 8, wherein the deamination is a chemical conversion by bisulphate.

10-11. (canceled)

12. The method of claim 1, wherein the first polymerase and/or the second polymerase is a DNA polymerase.

13. The method of claim 12, wherein the first polymerase is an archaeal DNA polymerase, or a modified archaeal DNA polymerase.

14. The method of claim 13, wherein the archaeal DNA polymerase, or modified archaeal DNA polymerase is Pfu DNA polymerase, Phusion DNA polymerase, Vent DNA polymerase, KOD DNA polymerase, Vent (exo-) DNA polymerase, Deep Vent (exo-) DNA polymerase, Deep Vent DNA polymerase, Q5, therminator DNA polymerase or any combination thereof.

15. The method of claim 1, wherein the first primer comprises a set of random primers, wherein the random primers comprise 3′ random sequence with or without 5′ universal tails, and wherein the first primer is capable of hybridising to any random regions.

16. The method of claim 1, wherein the first primer comprises a set of multiple target specific primers, wherein the primer sequence comprises a 3′ target specific sequence with or without a 5′ universal tail.

17. The method of claim 16, wherein the set of multiple target specific primers comprise a 3′ target specific sequence, an optional central series of nucleotides which is capable of acting as a unique molecular identifier, and a 5′ universal tail sequence, wherein the unique molecular identifier is of a suitable length and comprises a mixture of random nucleotides or degenerated nucleotides which allow for the identification of PCR duplicates in massively parallel sequencing.

18. The method of claim 17, wherein the 5′ universal tails comprise at least two different sequences for the opposing primers which flank a desired length of region to be amplified wherein the two opposing primers in proximity which flank an undesired length of region have the same universal tail sequence.

19. The method of claim 17, wherein primers in the set of multiple target specific primers comprise the same sequence of 5′ universal tails.

20. The method of claim 5, wherein the second primer comprises a second set of primers that comprises universal primers or/and target specific primers, wherein the universal primers comprise sequence identical or substantially identical to the 5′ tail sequences of the primers of the first set, wherein the target specific primers comprise 3′ target specific sequence and 5′ universal tails.

21. A method of preparing a sequencing library according to claim 1, the method comprising:
(a) providing a reaction mixture(s), each reaction mixture comprising nucleic acids to be sequenced, a first DNA polymerase, unusual nucleoside triphosphates and a first set of primers, wherein the polymerase is capable of extending primers using the target nucleic acids as templates and incorporating the unusual nucleotide into extension products which are modified complementary strands, and is incapable of efficiently making a copy using the modified complementary strand as template, wherein the unusual nucleoside triphosphate is distinct from the four standard nucleotides: deoxyadenosine triphosphate (dATP), deoxythymidine triphosphate (dTTP), deoxyguanosine triphosphate (dGTP), or deoxycytidine triphosphate (dCTP), and is capable of being incorporated into new strands, wherein the first set of primers comprise target specific primers, universal primers or random primers;
(b) performing extension reaction of primer and target nucleic acid template to produce modified complementary strands under extension condition, wherein the extension condition comprises buffer, any of four standard nucleoside triphosphates and appropriate temperature;
(c) optionally removing the nucleoside triphosphate and/or primers by purification or an enzymatic reaction;
(d) performing amplification of the modified complementary strands and/or original strands using a second set of primers and using a second DNA polymerase; and
(e) processing the products of step (d) to complete the library preparation for massive parallel sequencing.

22. The method of claim 21, wherein step (b) is a linear amplification by performing the extension once or more than once to produce multicopy of modified complementary strands.

23. A method of preparing a sequencing library for methylation analysis comprising:
(a) providing a reaction mixture(s), each reaction mixture comprising nucleic acids to be sequenced, a first DNA polymerase, unusual nucleoside triphosphates and a first set of primers, wherein the unusual nucleoside triphosphates is 5-Methyl-2′-deoxycytidine-5′-Triphosphate, wherein the polymerase is capable of extending primers using the target nucleic acids as templates and incorporating the unusual nucleotide into extension products which are modified complementary strands, wherein the first set of primers comprise target specific primers, universal primers or random primers;
(b) performing extension reaction of primer on target nucleic acid template to produce modified complementary strands under extension condition, wherein the extension condition comprises buffer, any of four standard nucleoside triphosphates and appropriate temperature;
(c) deaminating the DNA mixture by either chemical and/or enzymatic processes;
(d) purifying the DNA mixture;
(e) performing amplification of the DNA mixture using a second set of primers and using a second DNA polymerase; and
(f) processing the products of step (e) to complete the library preparation for massive parallel sequencing.

24. The method of claim 23, wherein step (e) the amplification comprises amplification of modified complementary strands and/or amplification of deaminated original strands or copies of deaminated original strands.

25. A kit for performing the method of claim 1, the kit comprising:
(a) a first DNA polymerase;
(b) one or more standard nucleotides: deoxyadenosine triphosphate (dATP), deoxythymidine triphosphate (dTTP), deoxyguanosine triphosphate (dGTP), and deoxycytidine triphosphate (dCTP);
(c) deoxyuridine triphosphate (dUTP) or 5-Methyl-2′-deoxycytidine-5′-Triphosphate;
(d) two or more primers- and
(e) a second DNA polymerase.

26. The method of claim 2, wherein the unusual nucleotide is 5-Methyl-2′-deoxycytidine-5′-Triphosphate,
wherein after step (b) the DNA mixture is deaminated by chemical and/or enzymatic processes,
wherein the modified complementary strands are protected from deamination, and
wherein the original strands are deaminated on the sites not methylated.

27. The method of claim 26, wherein the deamination is a chemical conversion by bisulphate.

28. The method of claim 26, wherein the modified complementary strands and/or the deaminated strands are amplified in step (d).

29. The method of claim 26, wherein after deamination and before step (d), the deaminated strands are linearly amplified with or without an unusual nucleotide.


SEQUENCE LISTING (11 sequences)

SEQ ID NO: 1 - DNA, 23 bp, Artificial Sequence
  Synthetic
acgcaggtcgtattgggcgcctg

SEQ ID NO: 2 - DNA, 24 bp, Artificial Sequence
  Synthetic
gggtcattgatggcaacaatatcc

SEQ ID NO: 3 - DNA, 25 bp, Artificial Sequence
  Synthetic
  misc_feature (1)..(1): Cyanine5 fluorescent dye
  misc_feature (25)..(25): Black Hole Quencher-2 dye
accagagttaaaagcagccctggtg

SEQ ID NO: 4 - DNA, 33 bp, Artificial Sequence
  Synthetic
  modified_base (32)..(33): the internucleotide linkage is a phosphorothioate bond
acactctttccctacacgacgctcttccgatct

SEQ ID NO: 5 - DNA, 70 bp, Artificial Sequence
  Synthetic
  modified_base (69)..(70): the internucleotide linkage is a phosphorothioate bond
aatgatacggcgaccaccgagatctacaccggaacaaacactctttccctacacgacgctcttccgatct

SEQ ID NO: 6 - DNA, 66 bp, Artificial Sequence
  Synthetic
  modified_base (64)..(66): the internucleotide linkages are phosphorothioate bonds
caagcagaagacggcatacgagatcattccaagtgactggagttcagacgtgtgctcttccgatct

SEQ ID NO: 7 - DNA, 49 bp, Artificial Sequence
  Synthetic
  misc_feature (35)..(49): n is a, c, g, t or u
  modified_base (48)..(49): the internucleotide linkage is a phosphorothioate bond
gtgactggagttcagacgtgtgctcuuccgaucunnnnnnnnnnnnnnn

SEQ ID NO: 8 - DNA, 48 bp, Artificial Sequence
  Synthetic
  misc_feature (34)..(48): n is a, c, g, t or u
  modified_base (47)..(48): the internucleotide linkage is a phosphorothioate bond
acactctttccctacacgacgctcuuccgaucunnnnnnnnnnnnnnn

SEQ ID NO: 9 - DNA, 36 bp, Artificial Sequence
  Synthetic
  misc_feature (22)..(36): n is a, c, g, t or u
  modified_base (35)..(36): the internucleotide linkage is a phosphorothioate bond
agacgtgtgctcttccgatctnnnnnnnnnnnnnnn

SEQ ID NO: 10 - DNA, 30 bp, Artificial Sequence
  Synthetic
ctctttccctacacgacgctcttccgatct

SEQ ID NO: 11 - DNA, 70 bp, Artificial Sequence
  Synthetic
agatcggaagagcgtcgtgtagggaaagagtgtttgttccggtgtagatctcggtggtcgccgtatcatt